(19) United States
(12) Patent Application Publication
(10) Pub. No.: US 2017/0081658 A1
(43) Pub. Date: Mar. 23, 2017

(54) Rapid Affinity Measurement of Peptide Ligands and Reagents Therefor

(71) Applicants: John CHAPUT, Phoenix, AZ (US); Andrew LARSEN, Scottsdale, AZ (US); Annabelle GILLIG, Tempe, AZ (US)

(72) Inventors: John CHAPUT, Phoenix, AZ (US); Andrew LARSEN, Scottsdale, AZ (US); Annabelle GILLIG, Tempe, AZ (US)

(21) Appl. No.: 15/367,255

(22) Filed: Dec. 2, 2016

Related U.S. Application Data
(62) Division of application No. 14/397,059, filed on Oct. 24, 2014.
(60) Provisional application No. 61/657,694, filed on Jun. 8, 2012.

(51) Int. Cl. C12N 15/10; C12N 15/67

(52) U.S. Cl. CPC C12N 15/1055; C12N 15/1062; C12N 15/67

(57) ABSTRACT

The present invention provides methods for rapidly screening and measuring the ligand binding affinity of in vitro selected peptides to the cognate and off-target proteins. This general strategy is amenable to high throughput analysis because the peptides are synthesized by cell-free translation, as opposed to solid-phase synthesis required by traditional assays, and affinities can be readily measured in standard formats.

### RELATED APPLICATIONS

This application claims priority to U.S. Provisional Patent Application Ser. No. 61/657694 filed Jun. 8, 2012, incorporated by reference herein in its entirety.

### STATEMENT OF GOVERNMENT RIGHTS

This invention was made with government support under DK093449 awarded by the National Institute of Health. The government has certain rights in the invention.

### BACKGROUND

Identifying peptides that bind to the surface of a protein with high affinity and high specificity is a time consuming process. In general, peptides are first selected from libraries of vast repertoires using any of a number of in vitro or in vivo selection technologies (i.e., DNA-display, phage display, mRNA display, ribosome display etc.). This process usually requires many cycles of selection and amplification, sometimes followed by additional rounds of directed evolution to optimize a given sequence for improved binding. The output of these selections are then cloned and sequenced, although in some cases, sequencing is done by mass spectrometry. Representative sequences are constructed by solid-phase synthesis, purified by HPLC, and assayed for affinity and specificity. Relative and specific solution binding affinities (Kd's) are typically measured on an individual basis using competitive binding assays or surface plasmon resonance (SPR). In total, this process is a costly endeavor that can easily take 2-3 months to complete per target.

Developing protein affinity reagents on a proteome-wide scale demands advances in peptide and protein selection technologies that reduce the time and cost required to generate and characterize high quality affinity reagents.

### SUMMARY OF THE INVENTION

In a first aspect, the present invention provides recombinant double stranded DNA constructs and nucleic acid libraries comprising a plurality of the recombinant double stranded DNA constructs, wherein each double stranded DNA construct comprises

(a) a promoter;

(b) one or more translation enhancement elements downstream of the promoter and upstream of the start codon;

(c) a start codon downstream of the one or more translation enhancing element;

(d) a random region of at least about 18 to about 60 nucleotides immediately downstream from the start codon;

(e) a protease cleavage site downstream of the random region;

(f) a unique restriction enzyme recognition site downstream of the protease cleavage site; and

(g) a heterologous cross-linking region downstream of the unique restriction enzyme recognition site.

In the libraries of this aspect of the invention, at least 10¹¹ different random sequences are represented in the plurality of double stranded nucleic acid constructs.

In one embodiment, expressed RNA from the cross-linking region can serve as a site for ligation to a linker containing a 3′-puromycin residue. In another embodiment, RNA expressed from the cross linking region is complementary to a DNA linker sequence to be used.

In a second aspect, the present invention provides recombinant double stranded DNA constructs and nucleic acid libraries comprising a plurality of the recombinant double stranded DNA constructs, wherein each double stranded DNA construct comprises

(a) a first restriction enzyme recognition site;

(b) one or more translation enhancement elements downstream of the first restriction enzyme recognition site;

(c) a start codon downstream of the one or more translation enhancement elements;

(d) a random region of at least about 18 to about 60 nucleotides immediately downstream from the start codon, wherein the peptide encoded by the random region of each linear recombinant double stranded DNA construct is capable of binding to the same target;

(e) a protease cleavage site downstream of the random region; and

(f) a second restriction enzyme recognition site downstream of the protease cleavage site.

In the libraries of this second aspect of the invention, at least 10 different random sequences are represented in the plurality of double stranded nucleic acid constructs.

In one embodiment, the double stranded DNA constructs comprises plasmids. In another embodiment, the recombinant double stranded DNA constructs further comprises:

(g) a promoter upstream of the first restriction enzyme recognition site; and

(h) a region encoding a peptide purification tag downstream of the second restriction enzyme recognition site.

In a third aspect, the invention provides methods for identifying polypeptide ligands for a target of interest, comprising

(a) contacting the recombinant nucleic acid constructs or nucleic acid library of any embodiment or combination of embodiments of the second aspect of the invention with reagents for RNA transcription under conditions to promote transcription of RNA from the double stranded nucleic acid constructs, resulting in an RNA expression product;

(b) contacting the RNA expression product with reagents for protein expression under conditions to promote translation of detectable polypeptide;

(c) incubating the detectable polypeptide with a target of interest under suitable conditions to promote binding of the detectable polypeptide to the target, to produce binding complexes; and

(d) analyzing the detectable polypeptides bound to the target.

In a fourth aspect, the invention provides methods for identifying peptide ligands for a target of interest, comprising

(a) contacting the recombinant nucleic acid constructs or the nucleic acid library of any embodiment or combination of embodiments of the first aspect of the invention with reagents for RNA transcription under conditions to promote transcription of RNA from the double stranded nucleic acid constructs, resulting in an RNA expression product;

(b) contacting the RNA expression product with reagents for ligating a linker containing a puromycin residue to the 3′ end of the RNA expression product, resulting in a labeled RNA expression product;

(c) contacting the labeled RNA expression product with reagents for protein expression under conditions to promote protein translation from the labeled RNA expression product, resulting in a RNA-polypeptide fusion product;

(d) reverse transcribing the RNA-polypeptide fusion products to produce an RNA-polypeptide fusion product-cDNA heteroduplex;

(e) incubating the RNA-polypeptide fusion product-cDNA heteroduplexes with a target of interest;

(f) removing RNA-polypeptide fusion product-cDNA heteroduplexes that are not bound to the target of interest, resulting in binding complexes; and

(g) amplifying ligand-bound RNA-polypeptide fusion product-cDNA heteroduplexes in the binding complexes, to produce double stranded DNA constructs that can be used to identify the peptide ligands bound to the target of interest.

In a fifth aspect, the present invention provides kits comprising

(a) the nucleic acid library of any embodiment or combination of embodiments of the first aspect of the invention; and

(b) an expression vector, wherein, the expression vector comprises:
(i) a promoter upstream of a first restriction enzyme recognition site; and
(ii) a region encoding a peptide purification tag downstream of a second restriction enzyme recognition site;
wherein the first and second restriction enzyme recognition sites are compatible with the unique restriction enzyme recognition site of the double stranded DNA constructs of the nucleic acid library.

In a sixth aspect, the invention provides separation devices, comprising:

(a) a multiwell plate;

(b) a regenerated cellulose layer below the multiwell plate, wherein the regenerated cellulose layer has a pore size suitable to retain peptides bound to a target, but not to retain unbound peptides; and

(c) a nylon membrane layer below the regenerated cellulose layer, wherein the nylon membrane layer has a pore size suitable to retain unbound peptides

In a seventh aspect, the invention provides an RNA pool resulting from transcription of the library of the first aspect or the second aspect of the invention.

### BRIEF DESCRIPTION OF THE FIGURES

FIG. 1. Schematic representation of the mRNA display library and the cell-free expression vector. The library (A) contains a T7 promoter for in vitro transcription [T7], followed by a translation enhancing element [TEE], followed by an ATG start codon, followed by a random region, followed by protease cleavage site [C.S.], followed by a restriction digest site [R.S.] and finally a photo-crosslinking site [X-Link]. Following selection the cDNA is amplified by PCR using a primer that adds a second restriction digest site at the 5′ end (B). Both the vector and the library are then digested using the same restriction enzymes so that they can be ligated back together (C). Colonies are then selected and purified vector will contain all the necessary genetic information for cell-free expression and purification of the peptide of interest (D).

FIG. 2. Cell-free expression and purification of selected peptides using customized vector as a template. In order to express peptides obtained from in vitro selection we use the plasmid vector as template for an in vitro transcription and translation reaction. This produces peptide with a C terminal peptide purification tag. After expression the lysate is passed through a purification column that binds the purification tag. Proteolytic cleavage releases the peptide of interest from the column for use in binding assays. This process does not require the peptide identity to be obtained by DNA sequencing in advance of completing binding studies. Peptide produced from as little as 10 μL of cell free expression lysate is sufficient to perform binding assays.

FIG. 3. Workflow for binding assays. To characterize the binding between a peptide and its cognate target the purified radiolabeled peptide is incubated with its target. After the system reaches equilibrium the sample is passed through a series of membranes in order to separate the bound and free peptides. This separation relies on the size difference between the free peptide and the peptide-target complex and is facilitated by a regenerated cellulose membrane that retains larger molecules while allowing the smaller free peptide to pass through. A nylon membrane then captures the free peptide and the amount of peptide on each membrane can be quantified by detection of the radiolabel.

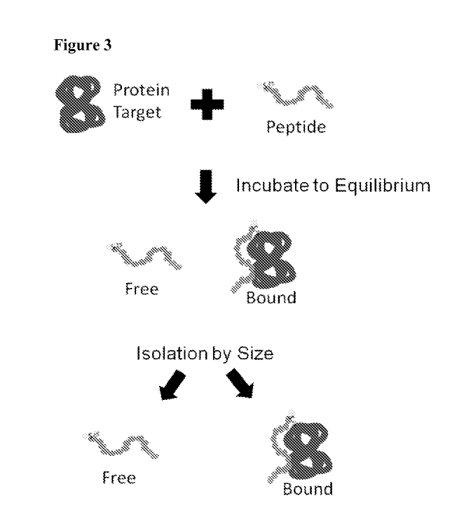

FIG. 4. Schematic of separation apparatus. Separation of the free and bound peptide is carried out in a 96-well apparatus where samples are loaded into individual wells and then passed through the membranes below.

### DETAILED DESCRIPTION OF THE INVENTION

In a first aspect, the present invention provides recombinant double stranded DNA constructs, or nucleic acid libraries comprising a plurality of the recombinant double stranded DNA constructs, wherein each double stranded DNA construct comprises

(a) a promoter;

(b) one or more translation enhancement elements downstream of the promoter and upstream of the start codon;

(c) a start codon downstream of the one or more translation enhancing elements;

(d) a random region of at least about 18 to about 60 nucleotides immediately downstream from the start codon;

(e) a coding region for a protease cleavage site downstream of the random region;

(f) a unique restriction enzyme recognition site downstream of the protease cleavage site; and

(g) a heterologous cross-linking region downstream of the unique restriction enzyme recognition site;

In the libraries of this aspect of the invention, at least 10¹¹ different random sequences are represented in the plurality of double stranded nucleic acid constructs.

The nucleic acid libraries according to all aspects of the present invention can be used, for example, in the methods of the invention for identifying peptide ligands for a target of interest. The libraries comprise a series of linear constructs, which, when used in in vitro selection methods as described herein, permit use of a library diversity of at least 10¹¹ different polynucleotide sequences. As used herein, a “library” is a collection of linear double stranded nucleic acid constructs.

As used herein, “heterologous” means that none of the promoter, translation enhancement element (TEE), random region, and cross-linking region are normally associated with each other (i.e.: they are not part of the same gene in vivo), but are recombinantly combined in the construct.

As used herein, a “promoter” is any DNA sequence that can be used to help drive RNA expression of a DNA sequence downstream of the promoter. Suitable promoters include, but are not limited to, the T7 promoter, SP6 promoter, CMV promoter, and vaccinia virus synthetic-late promoter. As will be understood by those of skill in the art, a given double stranded DNA construct may contain more than one promoter, as appropriate for a given proposed use.

As used herein, a translation enhancement element (TEE) can be any polynucleotide domain that mediates cap-independent protein translation. Any suitable TEE can be used, including but not limited to SEQ ID NO: 7-645, listed in Table 1. In a preferred embodiment, the isolated polynucleotides consist of the recited sequence. In a further embodiment, the isolated polynucleotides comprise the sequence of SEQ ID NO:4 (A/-)(A/G)ATC(A/G)(A/G)TAAA(T/C)G, wherein the isolated polynucleotides is between 13-200 nucleotides in length. SEQ ID NO:4 is a consensus sequence found within a number of the TEES (Clones 985 (SEQ ID NO:448), 1092 (SEQ ID NO:495), 1347 (SEQ ID NO:623), 906 (SEQ ID NO:408), 12 (SEQ ID NO:12), 1200 (SEQ ID NO:553), 958 (SEQ ID NO:434), 1011 (SEQ ID NO:458), 459 (SEQ ID NO:214) in Table 1). In a preferred embodiment, the isolated polynucleotides comprise the sequence of SEQ ID NO:5 5′-AAATCAATAAATG-3′, which is a conserved sequence found in the top-performing TEEs. In various preferred embodiments, the isolated polynucleotides are between 13-180, 13-170, 13-160, 13-150, 13-140, 13-130, 13-120, 13-110, 13-100, 13-90, 13-80, 13-70, 13-60, 13-50, 13-40, 13-30, or 13-20 nucleotides in length.

In one embodiment, the TEE is selected from the group consisting of SEQ ID NO:583 (clone 1267), SEQ ID NO:397 (clone 877), SEQ ID NO:54 (clone 100), SEQ ID NO:401 (clone 884), SEQ ID NO:471 (clone 1033), SEQ ID NO:327 (clone 733), SEQ ID NO:398 (clone 878), SEQ ID NO:301 (clone 675), and SEQ ID NO:310 (clone 694). In a further embodiment, the TEE comprises a nucleic acid sequence according to SEQ ID NO:1. This sequence represents a consensus sequence of a subset of 733 (SEQ ID NO:327), 877 (SEQ ID NO:397), 1033 (SEQ ID NO:471), and 1267 (SEQ ID NO:583), and thus is strongly correlated with TEE activity. In further embodiments, the TEE comprise a nucleic acid sequence according to SEQ ID NO:2 or SEQ ID NO:3, which are longer portions of the consensus sequence between 733 (SEQ ID NO:327), 877 (SEQ ID NO:397), 1033 (SEQ ID NO:471), 1267 (SEQ ID NO:583.

SEQ ID NO: 1:
5′AT(C/G)GAAT(C/G)(G/A)AA(G/T)(A/G/C)GAATGGA(A/T)
(A/T)(C/A/G)(A/G)AA(T/A)GGAAT(G/A)GAAT(T/G)(G/A)
AATGGAATGGAA(T/A)(T/G)GA(A/T)T(G/C)GAATG-3′
SEQ ID NO: 2:
5′-( [symbol] /--)( [symbol] /--)( [symbol] / [symbol] /--)( [symbol] / [symbol] /--)( [symbol] /--)
( [symbol] /--)( [symbol] /--)( [symbol] /--)( [symbol] /--)( [symbol] / [symbol] /--)(--/ [symbol] / [symbol] )
(-/ [symbol] )AT(C/G)GAAT(C/G)(G/A)AA(G/T)(A/G/C)
GAATGGA(AT)(A/T)(C/A/G)(A/G)AA(T/A)GGAAT(G/A)GAAT
(T/G)(G/A)AATGGAATGGAA(T/A)(T/G)GA(A/T)T(G/C)
GAATG-3′
SEQ ID NO; 3
5′-( [symbol] /--)( [symbol] /--)( [symbol] /--)( [symbol] / [symbol] /--)( [symbol] /--)( [symbol] /--)
( [symbol] /--)( [symbol] /--)( [symbol] /--)( [symbol] /--)(A/--)(A/--)(G/A/--)
(C/T/--)(G/--)(G/--)(A/--)(A/--)(T/--)(T/C/--)
(--/A/G)(--/A)AT(C/G)GAAT(C/G)(G/A)AA(G/T)(A/G/C)
GAATGGA(AT)(A/T)(C/A/G)(A/G)AA(T/A)GGAAT(G/A)GAAT
(T/G)(G/A)AATGGAATGGAA(T/A)(T/G)GA(A/T)T(G/C)
GAATG-3′

The “random region” is any DNA sequence of at least 18 nucleotides in length. In one embodiment, the random region is between 18-60 nucleotides in length. The random sequence may be non-naturally occurring, or derived from a naturally occurring source, and may be of any primary sequence.

As used herein, a “cross linking region” is any nucleic acid sequence that can be expressed as RNA, where the expressed RNA can serve as a site for ligation/binding to a linker to form a stable complex between mRNA-ribosome-protein. In a preferred embodiment, expressed RNA from the cross-linking region can serve as a site for ligation to a linker containing a 3′-puromycin residue. In a non-limiting embodiment, the expressed RNA from the cross-linking region can serve as a site for photo-ligation of a psoralen-DNA-puromycin linker (5′-psoralen-(oligonucleotide complementary to linker)-(PEG₉)₂-A₁₅-ACC-puromycin). In a preferred embodiment, the linker is a DNA linker, and the mRNA expressed from the cross linking region is complementary to the DNA linker sequence to be used.

The “protease cleavage site” can be the cleavage site for any suitable protease to be used in the methods of the invention.

The “unique restriction enzyme recognition site” can be any suitable restriction enzyme recognition site, so long as it is unique to the double stranded construct.

As used herein, “at least 10¹¹ different polynucleotide sequences are represented in the plurality of double stranded nucleic acid constructs” means that the library, in its entirety, contains at least 10¹¹ different polynucleotide sequences that can be tested for peptide binding activity to a target of interest, while each different double stranded nucleic acid construct contains only a single polynucleotide sequence. In various embodiments, at least 10¹², 10¹³, 10¹⁴, or 10¹⁵ different polynucleotide sequences are represented in the plurality of double stranded nucleic acid constructs.

It will be understood by those of skill in the art that the constructs of the invention may comprise further nucleotide elements as appropriate for a given intended use. In one preferred embodiment, the double stranded nucleic acid constructs further comprise one or more unique restriction sites upstream of the polynucleotide sequence and downstream of the promoter, and one or more unique restriction sites downstream of the polynucleotide sequence

In a second aspect, the present invention provides recombinant double stranded DNA constructs, and nucleic acid libraries that comprise a plurality of the recombinant double stranded DNA constructs, wherein each double stranded DNA construct comprises

(a) a first restriction enzyme recognition site;

(b) one or more translation enhancement elements downstream of the first restriction enzyme recognition site;

(c) a start codon downstream of the one or more translation enhancement elements;

(d) a random region of at least about 18 to about 60 nucleotides immediately downstream from the start codon, wherein the peptide encoded by the random region of each linear recombinant double stranded DNA construct is capable of binding to the same target;

(e) a coding region for a protease cleavage site downstream of the random region; and

(f) a second restriction enzyme recognition site downstream of the protease cleavage site;

wherein at least 10 different random sequences are represented in the plurality of double stranded nucleic acid constructs.

These constructs and libraries can be generated using any techniques, and can be used for identifying peptide ligands for a target of interest, such as disclosed in the methods of the invention. In another embodiment, the library of the first aspect of the invention is incubated with a desired target, washed to remove unbound peptides, and constructs encoding binding peptides to a specific target are amplified by PCR to isolate bound molecules. The linear DNA is restriction digested and cloned into a vector to create the nucleic acid libraries of this second aspect of the invention.

All terms used in this second aspect have the same meaning as used elsewhere herein; similarly, all embodiments of the nucleic acid libraries and components thereof that are disclosed above, and combinations thereof, can be used in the methods of the invention.

In one embodiment, the double stranded DNA constructs comprises plasmids. In another embodiment, the recombinant double stranded DNA constructs further comprises:

(g) a promoter upstream of the first restriction enzyme recognition site; and

(h) a region encoding a peptide purification tag downstream of the second restriction enzyme recognition site.

Any suitable region encoding a peptide purification tag can be used, as will be understood by those of skill in the art, based on the teachings herein. In one non-limiting and exemplary embodiment, the encoded purification tag may comprise streptavidin binding peptide.

The libraries of the third aspect of the invention comprise at least 10 different random sequences represented in the plurality of double stranded nucleic acid constructs. In various preferred embodiments, at least 15, 20, 25, 30, 35, 40, 45, 50, 75, 100, 250, 500, 1000, 2500, 5000, 10,000, 50,000, 100,000, or more different random sequences are represented in the plurality of double stranded nucleic acid constructs

It will be understood by those of skill in the art that the constructs of the invention may comprise further nucleotide elements as appropriate for a given intended use. In one preferred embodiment, the double stranded nucleic acid constructs further comprise one or more unique restriction sites upstream of the polynucleotide sequence and downstream of the promoter, and one or more unique restriction sites downstream of the polynucleotide sequence.

In a third aspect, the present invention provides methods for identifying polypeptide ligands for a target of interest, comprising

(a) contacting the nucleic acid library of any embodiment or combination of embodiments of the second aspect of the invention with reagents for RNA transcription under conditions to promote transcription of RNA from the double stranded nucleic acid constructs, resulting in an RNA expression product;

(b) contacting the RNA expression product with reagents for protein expression under conditions to promote translation of detectable polypeptide;

(c) incubating the detectable polypeptide with a target of interest under suitable conditions to promote binding of the detectable polypeptide to the target, to produce binding complexes; and

(d) analyzing the detectable polypeptides bound to the target.

The methods of the invention can be used, for example, to rapidly identify a plurality of peptides that bind to any target of interest. All terms used in this third aspect have the same meaning as used elsewhere herein; similarly, all embodiments of the nucleic acid libraries and components thereof that are disclosed above, and combinations thereof, can be used in the methods of the invention.

“Analyzing” the detectable polypeptides bound to the target means to make any qualitative or quantitative assessment of the bound polypeptide, including but not limited to determining a fraction of bound polypeptide, determining a binding constant of the bound polypeptide for the target, determining an amino acid sequence of the bound polypeptide, etc. The analyzing may further comprise purifying (partially or completely) bound polypeptide from the target.

The target may any target of interest, including but not limited to proteins, nucleic acids, lipids, polysaccharides, organic molecules, inorganic molecules, metals, polymers, solids, etc.

General conditions for in vitro transcription and translation are well known to those of skill in the art. Similarly, any suitable technique for detectably labeling the expressed polypeptides can be used, including but not limited to radioactive or fluorescent labeling, expressing the polypeptide a fusion protein with a detectable label, etc.

In a further embodiment, the target is immobilized on a solid support during the incubating step. Any suitable solid support can be used, including but not limited to magnetic beads, microarrays, columns, optical fibers, wipes, nitrocellulose, nylon, glass, quartz, diazotized membranes (paper or nylon), silicones, polyformaldehyde, cellulose, cellulose acetate, paper, ceramics, metals, metalloids, semiconductive materials, coated beads, magnetic particles; plastics such as polyethylene, polypropylene, and polystyrene; nanostructured surfaces; nanotubes (such as carbon nanotubes), and nanoparticles (such as gold nanoparticles or quantum dots).

In one embodiment, the target is incubated with an excess of the detectable polypeptide (i.e.: more than 1:1; preferably 1.5:1, 2:1, 3:1, 4:1, 5:1, 6:1, 7:1, 8:1, 9:1, 10:1, or more).

In embodiments where the constructs encode a peptide purification tag, the methods may further comprise passing the translation products through an affinity column with affinity for the peptide purification tag. Any suitable affinity column techniques can be used that permit binding to the peptide purification tag being used in a given method. It is well within the level of skill in the art, based on the teachings herein, to identify an appropriate affinity column technique to be used for a given purpose. In this embodiment, the methods may further comprise releasing isolated peptides from their purification tags, to help isolate the expressed peptide. It is well within the level of skill in the art, based on the teachings herein, to identify an appropriate release technique to be used for a given purpose. In a further embodiment, the methods may further comprise incubating the in vitro translated peptides with the target of interest to form a second binding complex, and removing unbound in vitro translated peptides. This embodiment helps to further purify the peptide binders of interest. Any suitable technique for removing unbound peptides can be used. In one non-limiting embodiment, removing unbound in vitro translated peptides comprises contacting the binding complexes with a size-limiting membrane, wherein detectable polypeptides bound to the target are retained on the membrane, and unbound polypeptides pass through pores of the membrane. Such membranes may be of any type that possesses suitable pore size, including but not limited to regenerated cellulose.

For example, the separation devices of the invention (see below) can be used for removal of unbound polypeptides. Combining various embodiments, radiolabeled peptides are brought to equilibrium with their cognate target, and the bound fraction is separated from the unbound fraction by passing the mixture through a size-limiting membrane. Peptides that are bound to a given target are retained on the top layer of regenerated cellulose, while unbound peptides are retained on the bottom layer of, for example, nylon. In a further embodiment, following separation, bound peptides can be quantitated using any suitable technique, including but not limited to phosphorimaging.

The methods of the invention provide a means, for example, to rapidly screen peptides identified in the output of an in vitro selection experiment. Traditionally, this was a costly and time consuming process that required generating each peptide by solid phase synthesis and measuring the properties of the peptide by a standard binding technique like SPR.

In a fourth aspect, the present invention provides methods for identifying peptide ligands for a target of interest, comprising

(a) contacting the nucleic acid library of any one embodiment or combination of embodiments of the first aspect of the invention with reagents for RNA transcription under conditions to promote transcription of RNA from the double stranded nucleic acid constructs, resulting in an RNA expression product;

(b) contacting the RNA expression product with reagents for ligating a linker containing a puromycin residue to the 3′ end of the RNA expression product, resulting in a labeled RNA expression product;

(c) contacting the labeled RNA expression product with reagents for protein expression under conditions to promote protein translation from the labeled RNA expression product, resulting in a RNA-polypeptide fusion product;

(d) reverse transcribing the RNA-polypeptide fusion products to produce an RNA-polypeptide fusion product-cDNA heteroduplex;

(e) incubating the RNA-polypeptide fusion product-cDNA heteroduplexes with a target of interest;

(f) removing RNA-polypeptide fusion product-cDNA heteroduplexes that are not bound to the target of interest, resulting in binding complexes; and

(g) amplifying ligand-bound RNA-polypeptide fusion product-cDNA heteroduplexes in the binding complexes, to produce double stranded DNA constructs that can be used to identify the peptide ligands bound to the target of interest.

The methods can be used, for example, to rapidly identify a plurality of peptides that bind to any target of interest. All terms used in this fourth aspect have the same meaning as used elsewhere herein; similarly, all embodiments of the nucleic acid libraries and components thereof that are disclosed above, and combinations thereof, can be used in the methods of the invention.

The target may any target of interest, including but not limited to proteins, nucleic acids, lipids, polysaccharides, organic molecules, inorganic molecules, metals, polymers, solids, etc.

In one embodiment of this fourth aspect, the double stranded DNA constructs comprise:

(a) a first restriction enzyme recognition site;

(b) one or more translation enhancement elements downstream of the first restriction enzyme recognition site;

(c) a start codon downstream of the one or more translation enhancement elements;

(d) a random region of at least about 18 to about 60 nucleotides immediately downstream from the start codon, wherein the peptide encoded by the random region of each linear recombinant double stranded DNA construct is capable of binding to the same target;

(d) a protease cleavage site downstream of the random region; and

(e) a second restriction enzyme recognition site downstream of the protease cleavage site. Any suitable embodiments or combinations thereof of the constructs as described above can be used in the methods of the invention.

General conditions for in vitro transcription and translation, PCR, reverse transcription, and mRNA display techniques are well known to those of skill in the art. Contacting the RNA expression product with reagents for ligating a linker containing a puromycin residue to the 3′ end of the RNA expression product, resulting in a labeled RNA expression product, can be carried out via any suitable method, including photo-crosslinking or Moore-Sharp splint-directed ligation. Any suitable linker may be used. In a preferred embodiment the linker comprises a DNA linker complementary to the transcribed single stranded RNA. The DNA linker may comprise any suitable modifications, including but not limited non-natural residues and pegylation, as can be used in mRNA display.

Similarly, general conditions for incubating the RNA-polypeptide fusion product-cDNA heteroduplexes with a target of interest; removing RNA-polypeptide fusion product-cDNA heteroduplexes that are not bound to the target of interest, resulting in binding complexes; and amplifying ligand-bound RNA-polypeptide fusion product-cDNA heteroduplexes in the binding complexes, to produce double stranded DNA constructs that can be used to identify the peptide ligands bound to the target of interest, are well known to those of skill in the art.

In a further embodiment, the target is immobilized on a solid support during the incubating step. Any suitable solid support can be used, including but not limited to magnetic beads, microarrays, columns, optical fibers, wipes, nitrocellulose, nylon, glass, quartz, diazotized membranes (paper or nylon), silicones, polyformaldehyde, cellulose, cellulose acetate, paper, ceramics, metals, metalloids, semiconductive materials, coated beads, magnetic particles; plastics such as polyethylene, polypropylene, and polystyrene;

nanostructured surfaces; nanotubes (such as carbon nanotubes), and nanoparticles (such as gold nanoparticles or quantum dots).

In one embodiment, the target is incubated with an excess of the RNA-polypeptide fusion product-cDNA heteroduplexes (i.e.: more than 1:1; preferably 1.5:1, 2:1, 3:1, 4:1, 5:1, 6:1, 7:1, 8:1, 9:1, 10:1, or more).

In another embodiment, removing RNA-polypeptide fusion product-cDNA heteroduplexes that are not bound to the target of interest comprises incubating the in the presence of a denaturant, including but not limited to guanidine hydrochloride, urea, and heat.

Traditionally, iterative rounds of in vitro selection and amplification are used to identify peptides with low nanomolar affinities to the surface of a given protein target. By combining the high library complexity of mRNA display with stringent washing conditions, we have discovered that high affinity peptides can be discovered without resorting to iterative rounds of selection and amplification. This advance greatly reduces the time required to generate and optimize high quality peptides.

In another embodiment, the methods further comprise cloning the double stranded DNA constructs that encode binders into an expression vector, wherein, after cloning, the vector comprises:

(g) a promoter upstream of the first restriction enzyme recognition site; and

(h) a region encoding a peptide purification tag downstream of the second restriction enzyme recognition site.

These added steps can be used, for example, to rapidly isolate the double stranded DNA constructs that encode peptide binders to a target of interest, and to use the isolated constructs to express the peptides of interest for isolation and identification.

Thus, in a further embodiment, the methods comprise in vitro translation of peptides encoded by the cloned double stranded DNA construct, wherein the peptides are expressed as N-terminal fusions with the peptide purification tag. Any suitable in vitro translation technique can be used. In one embodiment, the in vitro translation comprises use of a detectable amino acid monomer.

In a further embodiment, the methods comprise passing the in vitro translation products through an affinity column with affinity for the peptide purification tag. Any suitable affinity column techniques can be used that permit binding to the peptide purification tag being used in a given method. It is well within the level of skill in the art, based on the teachings herein, to identify an appropriate affinity column technique to be used for a given purpose. In this embodiment, the methods may further comprise releasing isolated peptides from their purification tags, to help isolate the expressed peptide. It is well within the level of skill in the art, based on the teachings herein, to identify an appropriate release technique to be used for a given purpose.

In a further embodiment, the methods may further comprise incubating the in vitro translated peptides with the target of interest to form a second binding complex, and removing unbound in vitro translated peptides. This embodiment helps to further purify the peptide binders of interest. Any suitable technique for removing unbound peptides can be used. In one non-limiting embodiment, removing unbound in vitro translated peptides comprises passing the second binding complex through a size-limiting membrane. Any suitable size-limiting membrane can be used, including but not limited to regenerated cellulose.

Combining various embodiments, radiolabeled peptides are brought to equilibrium with their cognate target, and the bound fraction is separated from the unbound fraction by passing the mixture through a size-limiting membrane. Peptides that are bound to a given target are retained on the top layer of regenerated cellulose, while unbound peptides are retained on the bottom layer of, for example, nylon.

In a further embodiment, following separation, bound peptides can be quantitated using any suitable technique, including but not limited to phosphorimaging.

In a fifth aspect, the present invention provides kits comprising

(a) the nucleic acid library of any embodiment or combination of embodiments of the first aspect of the invention; and

(b) an expression vector, wherein, the expression vector comprises:
(i) a promoter upstream of a first restriction enzyme recognition site; and
(ii) a region encoding a peptide purification tag downstream of a second restriction enzyme recognition site;
wherein the first and second restriction enzyme recognition sites are compatible with the unique restriction enzyme recognition site of the double stranded DNA constructs of the nucleic acid library.

Exemplary expression vectors include any embodiment or combination of embodiments of the vectors disclosed in the third aspect of the invention, and in the examples that follow. The library and vectors of the kits may independently be present on a solid surface or free in solution. The library and vectors of the kits may independently be frozen, lyophilized, or in solution.

In a sixth aspect, the present invention provides a separation device, comprising:

(a) a multiwell plate;

(b) a regenerated cellulose layer below the multiwell plate, wherein the regenerated cellulose layer has a pore size suitable to retain peptides bound to a target, but not to retain unbound peptides; and

(c) a nylon membrane layer below the regenerated cellulose layer, wherein the nylon membrane layer has a pore size suitable to retain unbound peptides.

The multiwell plate may comprise any number of wells as deemed appropriate by a user. The multiwell plate is one in which the wells are separated by barriers that allow peptides to pass through but retain proteins. In this way, peptides bound to a target may be retained on the regenerated cellulose layer, and peptides not bound to a target bind to the nylon membrane when passed through the wells of the multi-well plate.

In a seventh aspect, the present invention provides an mRNA pool resulting from transcription of the library of the nucleic acid library of the first aspect or the second aspect of the invention. Such mRNA pools can be used, for example, in the methods of the invention below. Any suitable technique for RNA transcription can be used. In one non-limiting embodiment, the double stranded DNA constructs each comprise a T7 RNA polymerase promoter, and the library is transcribed in vitro using T7 RNA polymerase, using standard techniques. It will be clear to those of skill in the art how to optimize transcription conditions in terms of buffers, nucleotides, salt conditions, etc., based on the general knowledge of in vitro transcription techniques in the art. The resulting mRNA pools will comprise single stranded RNA from all/almost all the double stranded DNA constructs in the library. In a further embodiment of mRNA pools resulting from transcription of the first aspect of the invention, the transcripts in the pooled mRNA comprise a DNA linker, containing a 3′ puromycin residue, ligated at the 3′ end of the transcript. In a further aspect, the invention provides pooled mRNA-peptide fusion molecules resulting from in vitro translation of the pooled mRNA. Methods for in vitro translation of RNA transcripts are well known to those of skill in the art. In one non-limiting embodiment, the methods comprise incubating the pooled mRNA with rabbit reticulocyte lysate and ³⁵S-methionine for a suitable time. The method may further comprise incubating the mixture overnight in the presence of suitable amounts of KCl and MgCl₂ to promote fusion formation. When the pool of RNA is translated in vitro, the product is an mRNA-peptide fusion molecule. The chemical bond forming step of mRNA display is due to the natural peptidyl transferase activity of the ribosome, which catalyzes the formation of a non-hydrolyzable amide bond between puromycin and the polypeptide chain. In this embodiment, individual RNA polynucleotides in the pool are covalently linked to a random peptide encoded by their random region. In a further embodiment, the RNA polynucleotides in the pool comprise RNA-cDNA heteroduplexes created via reverse transcription, as described in the methods that follow.

### EXAMPLES

We have developed methods, reagents, and device improvements that make it possible to select, sequence, and characterize high affinity peptides in days. This technology is automatable and could be performed in 96- or 384-well format. One specific embodiment of our technology is a custom library design and vector characterization strategy. A second embodiment is the use of a novel bar-coding strategy that is compatible with next-generation deep sequencing. Third, is a stringent selection strategy that reduces the number of selection cycles from many to one. Fourth, is a cell-free characterization process that allows for rapid screening and characterization of individual members without the need for solid-phase synthesis.

These advances make it possible to generate peptides with antibody-like affinity in 3-5 days. The process is amenable to automation and can be performed against tens-to-hundreds of proteins simultaneously. By combing in vitro selection with next-generation deep sequencing, it should be possible to map the ligand binding space for human and all other relevant proteomes.

### Specific Embodiments

1. Custom peptide library—vector characterization design strategy.

We have designed an mRNA display library and cell-free peptide expression vector that when used together make it possible to characterize selected peptides in 2-3 days.

This combined library—vector design strategy greatly reduces the time required to screen individual peptides present in the output of a protein selection. Traditionally, this is done by sequencing the selection output, synthesizing representative peptides by solid-phase synthesis, and purifying the polypeptides by HPLC. This is a time consuming process that can easily take 4-6 weeks. Even when the peptides are ordered from a commercial vendor, they can still take 3-4 weeks to receive and generally cost $200-300 per peptide depending on the level of purity requested.

In this specific embodiment, the library design strategy was made compatible with all of the sequence information needed to synthesize large peptide libraries by mRNA display; however, this strategy is general and could be applied to other selection technologies. The library was constructed at the DNA level and contains a T7 promoter for in vitro transcription, followed by a translation enhancing element, followed by an ATG start codon, followed by a random region, followed by protease cleavage site, followed by a restriction digest site and finally a photo-crosslinking site. Using standard mRNA display technology, the DNA library is transcribed into RNA, the RNA is photo-ligated to a short DNA fragment containing a 3′-puromycin residue. The library is translated in vitro to produce a library of peptides, each of which is covalently linked to their encoding RNA sequence. Prior to selection, the RNA portion of the mRNA-peptide fusion is reverse transcribed to create an RNA-cDNA heteroduplex.

The library is then incubated with a desired protein target, washed to remove unbound peptides, and amplified by PCR to isolate bound molecules. The linear DNA is restriction digested and cloned into our custom peptide expression vector. The custom protein expression vector contains a T7 promoter for in vitro transcription, followed by restriction sites that are compatible with the mRNA display library, followed by a peptide purification tag, followed by a PolyA region and finally a T7 terminator site. Individual clones are isolated by transforming the vector into Escherichia coli and picking individual colonies. Colonies are grown-up in LB or other suitable media and mini-prepped to isolate the vector.

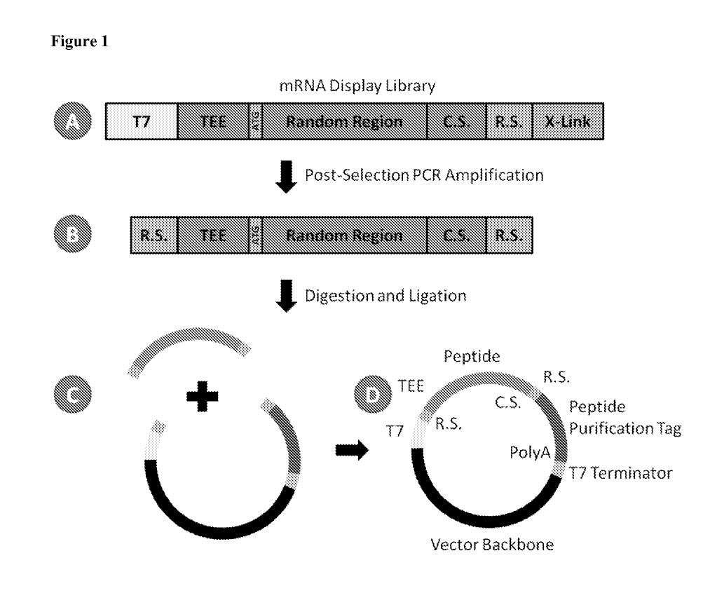

Each vector then serves as both a template for in vitro peptide expression and DNA sequencing (see FIG. 1).

To minimize the possibility of cross-contamination when multiple selections are conducted in parallel, multiple variants of the mRNA display library have been constructed and tested. These libraries are distinguished on the basis of their unique translation enhancing elements. In this way the libraries function almost identically under the same conditions, but can be discriminated by DNA sequencing. Development of these libraries also opens the opportunity for next-generation deep sequencing of multiple selection outputs at the same time. Such experiments make it possible to map the entire ligand binding space for a set of target proteins with very little investment of time or money.

### 2. Stringent Selection Strategy

We have developed methods and conditions that make it possible to identify peptides with antibody-like affinities (nM affinities) from a single mRNA display screen.

Traditionally, iterative rounds of in vitro selection and amplification are used to identify peptides with low nanomolar affinities to the surface of a given protein target. In general, in vitro selection technologies like mRNA display and ribosome display yielded higher affinity binders, because the starting libraries used for these technologies are much larger than what is commonly achieved with technologies that require transforming DNA into cells (i.e., cell-surface display or phage display).

By combining the high library complexity of mRNA display with stringent washing conditions, we have discovered that high affinity peptides can be discovered without resorting to iterative rounds of selection and amplification. This advance greatly reduces the time required to generate and optimize high quality peptides.

The first step of the selection is to immobilize the protein target to a solid support, such as a magnetic bead. The protein is then incubated with an excess of the peptide library, constructed as mRNA-peptide fusion molecules using standard mRNA display technology. Once equilibrium is achieved the beads are washed in selection buffer to remove all of the unbound peptide fusions. Next, the beads are incubated with selection buffer that includes denaturants such as guanidine hydrochloride. Subsequent rounds of washing will remove peptide that are weakly bound to the target protein, but retain all high affinity binders. Finally, the cDNA from the bound peptides are amplified using the polymerase chain reaction (PCR) and cloned into our cell-free expression vector.

### 3. Cell-Free Peptide Screening and Characterization

We have developed methods and devices that allow peptides present in the output of a selection to be rapidly screened and characterized in 1-2 days.

As described previously, peptides present in the output of a selection are typically synthesized by solid-phase synthesis and purified by HPLC. This is a time consuming process that is not easily amenable to high throughput automation and generally requires 3-4 weeks per peptide.

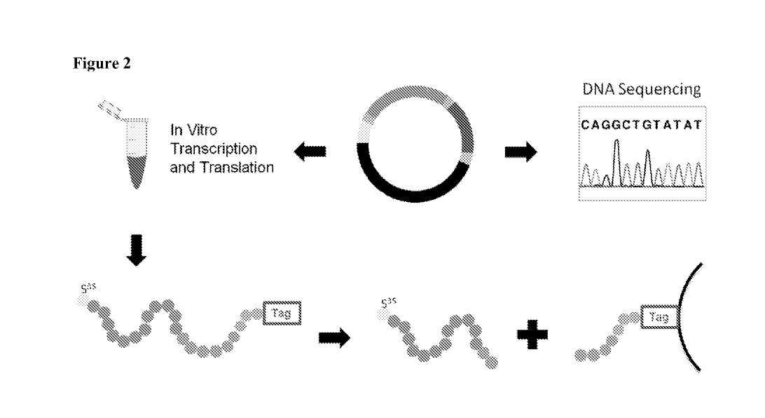

To eliminate this bottleneck, we have developed a custom peptide expression vector that allows peptides present in the output of a selection to be expressed in vitro as N-terminal fusions to a protein affinity tag. Sufficient peptide can be synthesized from less than 10 μL of cell-free expression lysate. Peptide expression is done in the presence of radiolabeled methionine, which allows the peptides to be detected by scintillation counting or phosphorimaging. Once expressed, peptides are purified by passing the crude lysate mixture through an affinity column with affinity to the peptide affinity tag. After washing the column, proteolytic cleavage then releases the peptide of interest from the purification tag. Alternatively, peptides can be recovered by incubating the beads in a suitable buffer like warm water or a competitive binder. Purified peptides are then used directly to evaluate different binders or obtain solution binding affinity (Kd) values for their cognate targets or off-target proteins (see FIG. 2).

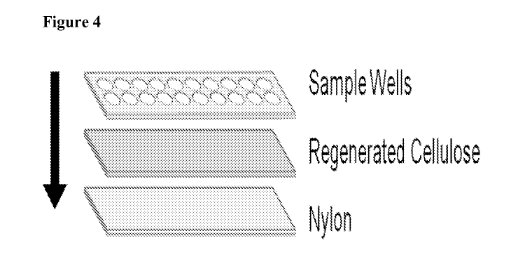

To determine the binding characteristics of each peptide, we developed a high throughput method and device that allows in vitro generated peptides to be rapidly and quantitatively screened for high affinity binding. With this method, radiolabeled peptides are brought to equilibrium with their cognate protein target, and the bound fraction is separated from the unbound fraction by passing the mixture through a size-limiting membrane. Peptides that are bound to a given target protein are retained on the top layer, while unbound peptides are retained on the bottom layer. Following separation, the amount of peptide on each membrane is quantification by phosphorimaging. The membranes used for our method include nylon, and regenerated cellulose. Regenerated cellulose has not previously been used in this way and therefore constitutes a new device (see FIG. 4). This method can be used to determine the binding affinity by running parallel reactions where the concentration of target protein is varied. Specificity measurements can be obtained by incubating peptides with non-target proteins.

We have validated the methods of the invention using two different peptides that are well characterized in the literature. The T10-39 peptide is a peptide selected to bind thrombin, while SBP is a peptide selected to bind streptavidin.

### In Vitro Peptide Expression and Purification

Peptides were expressed as fusions with a C-terminal affinity binding tag, the streptavidin binding peptide (SBP), using a coupled in vitro transcription/translation (TnT) rabbit reticulocyte lysate (Promega). One microgram of PCR-generate dsDNA was used as template in a 100 μL reaction that was spiked with ³⁵S-Methionine and left to incubate at 30° C. for 90 minutes. Expressed peptides were purified with 100 μL of streptavidin agarose loaded onto a column. The column was equilibrated with phosphate buffer saline (PBS) and the entire TnT lysate was loaded onto the column along with an equal volume of 2× PBS. The peptides were left on the column with shaking for 30 minutes at 4° C. to allow the peptides to bind. The column was then washed with PBS and peptides eluted in one of two ways. Peptides fused to the SBP tag were eluted as the full length construct with deionized water, or constructs containing a protease cleavage site between the peptide of interest and the affinity tag were incubated with the corresponding protease in order to elute the peptide of interest without the affinity tag. Elutions were monitored by liquid scintillation counting to identify the presence of peptides due to the incorporation of ³⁵S-Methionine during translation.

### Dot Blot Protocol

In order to determine the binding affinity of expressed peptides, multiple solutions were prepared that contain a constant amount of peptide and varying concentrations of target protein. These solutions were brought to equilibrium by incubating at 4° C. for one hour. Each solution of peptide and target protein was loaded into one well of a dot blot apparatus. The 96-well dot blot apparatus was prepared by building a stack of membranes that contains one piece of filter paper on the bottom, followed by two pieces of nylon membrane and topped with one piece of dialysis membrane. Once samples were loaded, vacuum was applied to the apparatus, pulling the solutions through the stack of membranes. Each membrane is imaged by phosphorimaging to detect signal from ³⁵S-Methionine, indicating which membranes have bound peptide. Free peptide passes through the dialysis membrane and binds to the nylon, while peptides bound to their target remain on the dialysis membrane once the solution is pulled through. The fraction of bound peptide for each concentration of target protein was used to plot a binding isotherm and determine the binding dissociation constant.

Data from these studies (not shown) demonstrated that the dissociation constants are consistent with literature values for these peptides (Raffler et al. Chemistry & Biology (2003) 10, 69-79; Wilson et al. Proceeding of the Nation Academy of Sciences (2001) 98, 3750-3755.)

TABLE 1
Clones sequenced for characterization after six rounds of mRNA display selection.

| Entry | Clone | Duplicates | Sequence |
|---|---|---|---|
| 1 | HGL6.1 | HGL6.346, | AATCGAATGGAATCAACATCAAACGGAAAAAAACGGAATTATCGAATGGAATCGAAGAGAATCATC |
| | | HGL6.670, | GAATGGACC (SEQ ID NO: 7) |
| | | HGL6.676, | |
| | | HGL6.715, | |
| | | HGL6.961, | |
| | | HGL6.1106, | |
| | | HGL6.1182, | |
| | | HGL6.1338 | |
| 2 | HGL6.5 | | TGGAATCGAATGGAATCAACATCAAACGGAAAAAAACGGAATTATCGAATGGAATCGAAGAGAATC |
| | | | ATCGAATGGACC (SEQ ID NO: 8) |
| 3 | HGL6.7 | | AGCATTCATATCTTGCAGTGTTGGGAAAGAGTGAGAGGTTGTGATGTCAAGAAGGATAGGTCAGAA |
| | | | GTGGAAGGTATGGGGGATTGTGCCTGCTGTCATGGCT (SEQ ID NO: 9) |
| 4 | HGL6.8 | | GGAACGAAATCGAATGGAACGGAATAGAATAGACTCGAATGTAATGGATTGCTATGTAATTGATTC |
| | | | GAATGGAATGGAATCGAATGGAATGCAATCCAATGGAATGGAATGCAATGCAATGAATGGAATGG |
| | | | AATGGAATGGAATGGAA (SEQ ID NO: 10) |
| 5 | HGL6.9 | | GGAACGAAATCGAATGGAACGGAATAGAATAGACTCGAATGTAATGGATTGCTATGTAATTGATTC |
| | | | GAATGGAATGGAATCGAATGGAATGCAATCCAATGGAATGGAATGCAATGCAATGAATGGAATGG |
| | | | AATGGAATGGAATGGA (SEQ ID NO: 11) |
| 6 | HGL6.12 | | TACGCAAATCGATAAATGTAATCCAGCATATAAACAGAACCAAAGACAAAAACCACATGATTATCTC |
| | | | AATAGATGCAGAAAAGGCC (SEQ ID NO: 12) |
| 7 | HGL6.14 | | ACTCGAATGCAATCAACATCAAACGGAATCAAACGGAATTATCGAATGGAATCGAAGAGAATCATC |
| | | | GAACGGACTCGAATGGAATCATCTAATGGAATGGAATGG (SEQ ID NO: 13) |
| 8 | HGL6.18 | | GAAATTCCAATTAAAATGAAATCGACTTATCTTAACAAATATAGCAATGCTGACAACACTTCTCCGGA |
| | | | TATGGGTACTGCT (SEQ ID NO: 14) |
| 9 | HGL6.20 | | AAGGAAAAGTAAAAGGAACTTAACACCTTCAAGAAAAGACAGACAAATAACAAAACAGCAGTTTGA |
| | | | TAGAATGAGATATCAGGGGATGGCA (SEQ ID NO: 15) |
| 10 | HGL6.21 | | ATCAACATCAAACGGAAAAAACGGAATTATCGAATGGAATCGAAGAGAATCATCGAACGGACC |
| | | | (SEQ ID NO: 16) |
| 11 | HGL6.22 | | AAAGAAAGACAGAGAACAAACGTAATTCAAGATGACTGATTACATATCCAAGAACATTAGATGGTC |
| | | | AAAGACTTTAAGAAGGAATACATTCAAAGGCAAAAAGTCACTTACTGATTTTGGTGGAGTTTGCCAC |
| | | | ATGGAC (SEQ ID NO: 17) |
| 12 | HGL6.23 | | AAGGGAATTGAATAGAATGAATCCGAATGGAATGGAATGGAATGGAATGGAATGGAATGGAATGG |
| | | | AATGGAATGGAATG (SEQ ID NO: 18) |
| 13 | HGL6.24 | | GAATGGAATCGAATCAAATTAAATCAAATGGAATGCAATAGAAGGGAATACAATGGAATAGAATG |
| | | | GAATGGAATGGAATGGACT (SEQ ID NO: 19) |
| 14 | HGL6.25 | | ACAGCAAGAGAGAAATAAAACGACAAGAAAACTACAAAATGCCTATCAATAGTTACTTTAAATATCA |
| | | | GTGGACCAAATCAGTGAAACAAAAGACACAGAGTGGC (SEQ ID NO: 20) |
| 15 | HGL6.27 | | TAGCAGGAAACAGCAAACTCAAATTAAGTAATTTCAAGAGCGTATCATCAATGAACTATTTTCAAAG |
| | | | ATGTGGGCAAGAT (SEQ ID NO: 21) |
| 16 | HGL6.28 | | AAACGGAATTATCAAATGGAATCGAAGAGAATCATCGAACGGACTCGAATGGAATCATCTAATGGA |
| | | | ATGGAATGGAAG (SEQ ID NO: 22) |
| 17 | HGL6.30 | | GAATGAAATGAAATCAAATNGAATGTACATGAATGGAATAGAAAAGAATGCATCTTTCTCGAACGG |
| | | | AAGTGCATTGAATGGAAAGGAATCTACTGGAATGGATTCGAATGGAATGGAANGGGATGGAATGG |
| | | | TATGG (SEQ ID NO: 23) |
| 18 | HGL6.32 | | AATGGACTCGAATGAAATCATCATCAAACGGAATCGAATGGAATCATTGAATGGAAAGGATGGGAT |
| | | | CATCATGGAATGGAAACGAATGGAATCACTG (SEQ ID NO: 24) |
| 19 | HGL6.34 | | AATGGAATCATTGAATGGAATGGAATGGAATCATCAAAGAAAGGAATCGAAGGGAATCATCGAAT |
| | | | GGAATCAAACGGAATCATCGAATGGAATGGAATGGAATG (SEQ ID NO: 25) |
| 20 | HGL6.38 | HGL6.537 | AGCAGAAGAAATAACTGAAATCAGAGTGAAACTGAATCAAATTGAGATGCAAAAATACATACGAAA |
| | | | TGGCCAG (SEQ ID NO: 26) |
| 21 | HGL6.40 | | AGTTAATCCGAATAGAATGGAATGGAATGCAATGGAACGGAATGGAACGGAATGGAATGGAATGG |
| | | | AATGGAATGGAATG (SEQ ID NO: 27) |
| 22 | HGL6.42 | | ATGGAATCAACATCAAACGGAATCAAACGGAATTATCGAATGGAATCGAAGAGAATCATCGAACGG |
| | | | ATTCGAATGGAATCATCTAATGGAATGGAATGGAAGAATCCATGGACTCGAATGCAATCATCAGCG |
| | | | AATGGAATCGAATGGAATCATCGAATGGACTCG (SEQ ID NO: 28) |
| 23 | HGL6.44 | | AAAGGAATGGACTGGAACAAAATGAAATCGAACGGTAGGAATCGTACAGAACGGACAGAAATGGA |
| | | | ACGGCATGGAATGCACTCG (SEQ ID NO: 29) |
| 24 | HGL6.47 | | AAATCAACAACAAACGGAAAAAAAAGGAATTATCGAATGGAATCAAAGAGAATCATCGAATGGACC |
| | | | (SEQ ID NO: 30) |
| 25 | HGL6.50 | | AAATGAACAAAACTAGAGGAATGACATTACCTGACTTCAAATTATACTACAGAGCTATAGTAACCAA |
| | | | AACAGCATGGTACAGGCAT (SEQ ID NO: 31) |
| 26 | HGL6.51 | | GTAATGGAATGGAATGGAAAGGAATCGAAACGAAAGGAATGGAGACAGATGGAATGGAATGGAA |
| | | | CAGAG (SEQ ID NO: 32) |
| 27 | HGL6.52 | HGL6.496, | ATCGAATGGAATCAACATCAAACGGAAAAAAACGGAATTATCGAATGGAATCGAAGAGAATCATCG |
| | | HGL6.881, | AATGGACC (SEQ ID NO: 33) |
| | | HGL6.1207 | |
| 28 | HGL6.57 | | CAATCAGAGCGGACACAAACAAATTGCATGGGAAGAATCAATATCGTGAAAATGGCC (SEQ ID NO: 34) |
| 29 | HGL6.59 | | AGACCTTTCTCAGAAGACACACAAATTGCCAACAGGTATATGAAAAAATGTTCAATATCACTAATCA |
| | | | TCAGGGCGATGCC (SEQ ID NO: 35) |
| 30 | HGL6.61 | | CATGGAATCGAATGGAATTATCATCGAATGGAATCGAATGGTACCAACACCAAACGGAAAAAAACG |
| | | | GAATTATCGAATGGAATCGAAGAGAATCTTCGAACGGACC (SEQ ID NO: 36) |
| 31 | HGL6.63 | | GAACGATTTATCACTGAAAATTAATACTCATGCAAGTAGTAAACGAATGTAATGACCATGATAAGGA |
| | | | GACGGACGGTGGTGATAGT (SEQ ID NO: 37) |
| 32 | HGL6.65 | | AAAGATCAANGNNCAAAAATCAGCAGCATTTCTATAAACCAACAATGTCCAGGCTGAGAGNGAAAT |
| | | | CAAGAAANCAATTC (SEQ ID NO: 38) |
| 33 | HGL6.66 | | ACACACATACCAACAGAACATGACAAAAGAACAAAACCAGCCGCATGCATACTCGATGGAGACAAA |
| | | | GGTAACACTGCAGAATGGTGAAGGAAGAACAGTCATTTTAATGACAGTGTTGGCT (SEQ ID NO: 39) |
| 34 | HGL6.67 | HGL6.463, | AATGGAATCAACATCAAACGGAAAAAAACGGAATTATCGAATGGAATCGAAGAGAATCATCGAATG |
| | | HGL6.775, | GACC (SEQ ID NO: 40) |
| | | HGL6.936 | |
| 35 | HGL6.68 | | ATCAAAAGGAACGGAATGGAATGGAATGGAATGGAATGGAATGGAATGGAATGGAATGAAATCAA |
| | | | CCCGAATGGAATGGATTGGCATAGAGTGGAATGG (SEQ ID NO: 41) |
| 36 | HGL6.70 | HGL6.71 | TAAAGAAAAACAAACAAACAGAAATCAATGAAAATCCCATTCAAAGGTCAGCAACCTCAAAGACTG |
| | | | AAGGTAGATAAGCCCACAAGGATG (SEQ ID NO: 42) |
| 37 | HGL6.73 | | AAACGGAAAAAAACGGAATTATCGAATGGAATCGAATAGAATCATCGAATGGACC (SEQ ID NO: 43) |
| 38 | HGL6.74 | | GGAATCAACTCGATTGCAATGGAATGCAATGGAAAGGAATGGAATGCAATTAAAGCGAATAGAAT |
| | | | GGAATGGAATGGAATGGAACGGAATGGAATG (SEQ ID NO: 44) |
| 39 | HGL6.76 | | GAAGAAGAAAAAACATGGATATACAATGTCAACAGAAATCAAGGAGAAACGGAATTTCACCAATCA |
| | | | ATTTAGTGATCTGGGTT (SEQ ID NO: 45) |
| 40 | HGL6.82 | | TGGAATCATCTAATGGAATGGAATGGAATAATCCATGGACTCGAATGCAATCATCATAAAATGGAAT |
| | | | CGAATGGAATCAACATCAAATGGAATCAAATGGGATCATTGAACGGAATTGAATGGAATCGTCAT |
| | | | (SEQ ID NO: 46) |
| 41 | HGL6.83 | | TGAACAGAGAATTGGACAAAACGCACAAAGTAAAGAAAAAGAATGAAGCAACAAAAGCAGAGATT |
| | | | TATTGAAAACAAAAGTACACACCACACAGGGTGGGAGTGG (SEQ ID NO: 47) |
| 42 | HGL6.85 | HGL6.980, | GGAATCAACATCAAACGGAAAAAAACGGAATTATCGAATGGAATCGAAGAGAATCATCGAATGGA |
| | | HGL6.1002 | CC (SEQ ID NO: 48) |
| 43 | HGL6.88 | | AACACGACTTTGAGAAGAGTAAGTGATTGTTAATTAAAGCAAGAGAATTATTGATGTATCACAGTCA |
| | | | TGAGAAATATTGGAAGGAATATGGTCCATAC (SEQ ID NO: 49) |
| 44 | HGL6.91 | | TGAAAAGAAGAATGACCATAAGCAAGCAGATGAAAAACAAAACAGAATTTTTACAGACGTCTTGGA |
| | | | CTGATATCTTGGGC (SEQ ID NO: 50) |
| 45 | HGL6.92 | | AATCAATAAATGTAAACCAGCATATAAACAGAACCAACGACAAAAACCACATGATTATCTCAATAGA |
| | | | TGCAGAAAAGGCC (SEQ ID NO: 51) |
| 46 | HGL6.95 | | CAACATCAAACGGAATCAAACGGAATTATCGAATGGAATCGAAGAGAATCATCGAATGGACTCGAA |
| | | | TGGAATCATCTAATGGAATGGAATGGAAG (SEQ ID NO: 52) |
| 47 | HGL6.96 | | AATGGAAGGGAATGGAATGGAATCGAATCGAATGGAACAGAATTCAATGGAATGGAATGGAATGG |
| | | | AATGGAATCGAATGGAATGG (SEQ ID NO: 53) |
| 48 | HGL6.100 | | AAAGACTTAAACATAAGACCTAAAACCATAAAAACCACAGAAGAAAACATAGGCAATGCCATTCAG |
| | | | GACATAGGCATGGGCAAAGACTTC (SEQ ID NO: 54) |
| 49 | HGL6.101 | | AGACTTGAAAAGCACAGACAACGAAAGCAAAAATGGACAAATGGAATCACATCAAGCTAAAAGGTT |
| | | | TTGCATGGCAAAGG (SEQ ID NO: 55) |
| 50 | HGL6.112 | HGL6.952, | AGCAACTTCAGCAAAGTCTCAGGATACAAAATCAATGTGCAAAAATCACAAGCATTCTTATACACCA |
| | | HGL6.955 | ACAACAGACAAACAGAGAGCC (SEQ ID NO: 56) |
| 51 | HGL6.113 | | TGAATGCTATAGAGCAGTAAAAACAAATAAATGAACTACATTACAGCTACTTACAACCATATGAAAG |
| | | | AATATAACCATAACAATGATGAGTGGACAAAAGCTAAGTGTGAAAGAATGCATAGTGCTACAGCAG |
| | | | CCAACATTTACAGC (SEQ ID NO: 57) |
| 52 | HGL6.115 | | AACAAAATTGAACAACATGCAAAGAAACATAAACGAAGCAATGAAAGTGTGCAGATCCACTGAAAT |
| | | | GAAAGTGCTGTCCAGAGTGGGAGCCAGCTCGAGA (SEQ ID NO: 58) |
| 53 | HGL6.116 | | TGGAATTATCGTCGAATAGAATCGAATGGTATCAACATCAAACGGAAAAAAACGGAATTATCGAAT |
| | | | GGAATCGAAGAGAATCATCGAACGGACTCGAATGGAATCATCTAATGGAATGGAATGGAATAATCC |
| | | | ATGG (SEQ ID NO: 59) |
| 54 | HGL6.117 | | AGATAAGTGGATGAACAGATGGACAGATGGATGGATGGATGGATGGATGGATGGATGCCTGGAA |
| | | | GAAAGAAGAATGGATAGTAAGCTGGGTATA (SEQ ID NO: 60) |
| 55 | HGL6.119 | | AATCAAAGAATTGAATCGAATGGAATCATCTAATGTACTCGAATGGAATCACCAT (SEQ ID NO: 61) |
| 56 | HGL6.121 | | AATGGAATCGAACGGAATCATCATCAAACGGAACCGAATGGAATCATTGAATGGAATCAAAGGCAA |
| | | | TCATGGTCGAATG (SEQ ID NO: 62) |
| 57 | HGL6.122 | | AGGAATCTATAATACAGCTGTTTATAGCCAAGCACTAAATCATATGATACAGAAAACAAATGCAGAT |
| | | | GGTTTGAAGGGTGGG (SEQ ID NO: 63) |
| 58 | HGL6.125 | | AACGGAAAAAAACGGAATTATCGAATGGAATCGAAGAGAATCATCGAATGGACC (SEQ ID NO: 64) |
| 59 | HGL6.126 | | TGAGAAAATGATGGAAAAGAGGAATAANACGAAACAAAACCACAGGAACACAGGTGCATGTGAAT |
| | | | GTGCACAGACAAAGATACAGGGCGGACTGGGAAGGAAGTTTCTGCACCAGAATTTGGGG (SEQ ID |
| | | | NO: 65) |
| 60 | HGL6.132 | | AATGGAATCGAAGAGAATGGAAACAAATGGAATGGAATTGAATGGAATGGAATTGAATGGAATGG |
| | | | GAAGGAATGGAGTG (SEQ ID NO: 66) |
| 61 | HGL6.134 | | AATGTCAAGTGGAATCGAGTGGAATCATCGAAAGAAATCGAATGGAATCGAAGGGAATCATTGGA |
| | | | TGGGCTCAAAT (SEQ ID NO: 67) |
| 62 | HGL6.137 | | AAACAATGGAAGATAATGGAAAGATATCGAATGGAATAGAATGGAATGGAATGGACTCAAATGGA |
| | | | ATGGACTTTAATGGAATGG (SEQ ID NO: 68) |
| 63 | HGL6.138 | | GAACAATCAATGGAAGCAGAAACAAATAAACCAAGGTGTGCATCAAGGAATACATTCACGCATGAT |
| | | | GGCTGTATGAGTAAAATG (SEQ ID NO: 69) |
| 64 | HGL6.139 | | AAACCGAATGGAATGGAATGGACGCAAAATGAATGGAATGGAAGTCAATGGACTCGAAATGAATG |
| | | | GAATGGAATGGAATGGAATG (SEQ ID NO: 70) |
| 65 | HGL6.140 | | AGGATACAAAATCAAAGTGCAAAAATCACAAGCATTCTTATACACCAATAACAGACAAACAGAGAG |
| | | | CC (SEQ ID NO: 71) |
| 66 | HGL6.147 | | GGAATCGAATGGAATCAACATCAAACGGAAAAAAACAGAATTATCGTATGGAATCGAATAGAATCA |
| | | | TCGAATGGACC (SEQ ID NO: 72) |
| 67 | HGL6.148 | | CAACCCGAGTGGAATAAAATGGAATGGAATGGAATGAAATGGAATGGATCGGAATGGAATCCAAT |
| | | | GGAATCAACTGGAATGGAATGGAATGGAATG (SEQ ID NO: 73) |
| 68 | HGL6.149 | | TATCATCGAATGGAATCGAATGGAATCAACATCAAACGGAAAAAAACGGAATTATCGAATGGAATC |
| | | | GAAGAGAATCATCGAATGGACC (SEQ ID NO: 74) |
| 69 | HGL6.150 | | CGGAATAATCATTGAACGGAATCGAATGGAATCATCATCGGATGGAAACGAATGGAATCATCATCG |
| | | | AATGGAAATGAAAGGAGTCATC (SEQ ID NO: 75) |
| 70 | HGL6.151 | | CAACACACAGAGATTAAAACAAACAAACAAACAATCCAGCCCTGACATTTATGAGTTTACAGACTGG |
| | | | TGGAGAGGCAGAGAAG (SEQ ID NO: 76) |
| 71 | HGL6.152 | | GGAATGGAATGAACACGAATGTAATGCAACCCAATAGAATGGAATCGAATGGCATGGAATATAAA |
| | | | GAAATGGAATCGAAGAGAATGGAAACAAATGGAATGGAATTG (SEQ ID NO: 77) |
| 72 | HGL6.153 | | CACTACAAACCACGCTCAAGGCAATAAAAGAACACAAACAAATGGAAAAACATTCCATGCTCATGG |
| | | | ATGGG (SEQ ID NO: 78) |
| 73 | HGL6.158 | | AATCGAATGGAATTAACATCAAACGGAAAAAAACGGAATTATCGAATGGAATCGAAGAGAATCATC |
| | | | GAATGGACC (SEQ ID NO: 79) |
| 74 | HGL6.161 | | TGGAAAAGAATCAAATTGAATGGCATCGAACGGAATGGGATGGAATGGAATAGACCCAGATGTAA |
| | | | TGGACTCGAATGGAATG (SEQ ID NO: 80) |
| 75 | HGL6.163 | | AATCAGTCTAGATCTTAAAGGAACACCAGAGGGAGTATTTAAATGTGCCCAATAAGCAAGAATTAT |
| | | | GGTGATGTGGAAGTA (SEQ ID NO: 81) |
| 76 | HGL6.164 | | CCATAACACAATTAAAAACAACCTAAATGTCTAATAGAAGAACACTGTTCAGACCGGGCATGGTGGC |
| | | | TTATACC (SEQ ID NO: 82) |
| 77 | HGL6.165 | | GACTAATATTCAGAATATACAAGGAACTCAAACAACTCAACAGTAGAAAAAAAAACCTGAATAGAC |
| | | | ATTTCTCAAAAGAAGACATACAAATGGCC (SEQ ID NO: 83) |
| 78 | HGL6.171 | HGL6.1149 | AACAGACCATAAATAAACACAGAAGACACACGAGTGTAAAGTCAGTGCCCCGCTGCGAATTAAATC |
| | | | GGGGTGATGTGATGGCGAGTGAGTGGGTAGTT (SEQ ID NO: 84) |
| 79 | HGL6.174 | | ATCATTGAATGCAATCACATGGAATCATCACAGAATGGAATCGTACGGAATCATCATCGAATGGAAT |
| | | | TGAATGGAATCATCAATTGGACTCGAATGGAAACATCAAATGGAATCGATTGGAAGTGTCGAATGG |
| | | | ACTCG (SEQ ID NO: 85) |
| 80 | HGL6.175 | | GGTCCATTCGATGATTCTCTTCGATTCCATTCGATAATTCCGTTTTTTCCCGTTTGATGTTGATTCC |
| | | | (SEQ ID NO: 86) |
| 81 | HGL6.178 | | AGCAACTTCAGTAAAGTGTCAGGATACAAAATCAATGTGCAAAAATCACAAGCATTCTTATACATCA |
| | | | ATAACAGACAAACAGAGAGCCAAA (SEQ ID NO: 87) |
| 82 | HGL6.180 | | AGCAACTTCAGCAAAGTCTCAGGATACAAAATCAATGTGCAAAAATCACAAGCATTCCTATACACCA |
| | | | ACAACAGACAAACAGAGAGCC (SEQ ID NO: 88) |
| 83 | HGL6.181 | | GAATAATCATTGAACGGAATCGAATGGAATCATCATCGGATGGAAACGAATGGAATCATCATCGAA |
| | | | TGGAAATGAAAGGAGTCATC (SEQ ID NO: 89) |
| 84 | HGL6.182 | HGL6.902 | TAATCATCTTCGAATTGAAAACAAAGCAATCATTAAATGTACTCTAACGGAATCATCGAATGGACC |
| | | | (SEQ ID NO: 90) |
| 85 | HGL6.184 | HGL6.1215 | GGAATCGAATGGAATCAACATCAAACGGAAAAAAACGGAATTATCGAATGGAATCGAAGAGAATC |
| | | | ATCGAATGGACC (SEQ ID NO: 91) |
| 86 | HGL6.186 | | GATCAGCTTAGAATACAATGGAACAGAACAGATTAGAACAATGTGATTTTATTAGGGGCCACAGCA |
| | | | CTGTTGACTCAAGTACAAGTTCTGACTCATGTAGAACTAACACTTTT (SEQ ID NO: 92) |
| 87 | HGL6.187 | | AGAGAAAAGATGATCATGTAACCATTGAAAAGACAATGTACAAAACTAATACTAATCACACAGGAC |
| | | | CAGAAAGCAATTTAGACCAT (SEQ ID NO: 93) |
| 88 | HGL6.190 | | AATGGAATCGAATGGAATCAACATCAAACGGAAAAAACGGAATTATCGAATGGAATCAAAGAGAAT |
| | | | CATCGAATGGACC (SEQ ID NO: 94) |
| 89 | HGL6.191 | | AATGGAATTATCATCGAATGGAATCGAATGGAATCAACATCAAACGGAAAAAAACGGAATTATCGA |
| | | | ATGGAATCGAAGAGAATCATCGAATGGACC (SEQ ID NO: 95) |
| 90 | HGL6.197 | | GTCAACACAGGACCAACATAGGACCAACACAGGGTCAACACAGGACCAACATAGGACCAACACAG |
| | | | GGTCAACACAAGACCAACATGGGACCAACACAGGGTCAACATAGGACCAACATGGGACCAACACA |
| | | | GGGTCAACACAGGACCAAC (SEQ ID NO: 96) |
| 91 | HGL6.198 | | TATAGTTGAATGAACACACATACACACACACATGCCACAAAACAAAAACAAAGTTATCCTCACACAC |
| | | | AGGATAGAAACCAAACCAAATCCCAACACATGGCAAGATGAT (SEQ ID NO: 97) |
| 92 | HGL6.206 | | GAATCAACTCGATTGCAATCGAATGGAATGGAATGGTATTAACAGAATAGAATGGAATGGAATGGA |
| | | | ATGGAACGGAACG (SEQ ID NO: 98) |
| 93 | HGL6.208 | | AATGGAATGGAATAATCGACGGACCCGAATGCAATCATCATCGTACAGAATCGAATGGAATCATCG |
| | | | AATGGACTGGAATGGAATGG (SEQ ID NO: 99) |
| 94 | HGL6.210 | | AATACAAACCACTGCTCAACGAAATAAAAGAGGATACAAACAAATGGAAGAACATTCTATGCTCAT |
| | | | GGGTAGGATGAATTCATATCGTGAAAATGGCCATACTGCC (SEQ ID NO: 100) |
| 95 | HGL6.215 | | AAACACGCAAACACACACACAAGCACACTACCACACAAGCGGACACACATGCAAACACGCGAACAC |
| | | | ACACACATATACACACAAGCACATTACAAAACACAAGCAAACACCAGCAGACACACAAACACACAA |
| | | | ACATACATGG (SEQ ID NO: 101) |
| 96 | HGL6.219 | | AATCGAACGGAATCAACATCAAACGGAAAAAAAACGGAATTATCGAATGGAATCGAAGAGAATCAT |
| | | | CGAATGGACC (SEQ ID NO: 102) |
| 97 | HGL6.220 | HGL6.301, | ACACATTTCAAGGAAGGAAACAAGAACAGACAGAAACACAACATACTTCATGAAACCACATTTTAGC |
| | | HGL6.1353 | ATCCTGGCCGAGTATTCATCA (SEQ ID NO: 103) |
| 98 | HGL6.222 | | GGATACAAAATCAATGTACAAAAATCACAAGCATTCTTATACACCAATAACAGACAAACAGAGAGCC |
| | | | (SEQ ID NO: 104) |
| 99 | HGL6.223 | | TAATTGATTCGAATGGAATGGAATAGAATGGAATTGAATGGAATGGACCATAATGGATTGGACTTT |
| | | | AATAGAAAGGGCATG (SEQ ID NO: 105) |
| 100 | HGL6.225 | | AGCAACTTCAGCAAAGTCTCAGGATACAAAATCAATGTACAAAAGTCACAAGCATTCTTATACACCA |
| | | | ACAAAAGACAAACAGAGAGCC (SEQ ID NO: 106) |
| 101 | HGL6.228 | | ACATCAAACGGAAAAAAAAAACAAAACGGAATTATCGAATGGAATCGAAGAGAATCATCGAATGG |
| | | | ACC (SEQ ID NO: 107) |
| 102 | HGL6.229 | | ACATCTCACTTTTAGTAATGAACAGATCATTCAGACAGAAAATTAGCAAAGAAACATCAGAGTTAAA |
| | | | CTACACTCTAAACCAAATGGACCTA (SEQ ID NO: 108) |
| 103 | HGL6.231 | | GAAGAAAGCATTCATTCAAGACATCTAACTCGTTGATATAATGCATACAGTTCAAAATGATTACACTA |
| | | | TCATTACATCTAGGGCTTTC (SEQ ID NO: 109) |
| 104 | HGL6.232 | | GCAAAAGAAACAATCAGTAGAGTAAACAGACAACTCATAGAATGCAAGAAAATCATCGCAATCTGT |
| | | | ACATCCAACAAAGGGCT (SEQ ID NO: 110) |
| 105 | HGL6.235 | | ACACACACATTCAAAGCAGCAATATTTACAACAGCCAAAAGGTGGAAACAATTGAGCAATTG (SEQ |
| | | | ID NO: 111) |
| 106 | HGL6.237 | | ATCATCGAATAGAATCGAATGGTATCAACACCAAACGGAAAAAAACGGAATTATCGAATGGAATCG |
| | | | AAGAGAATCTTCGAACGGACC (SEQ ID NO: 112) |
| 107 | HGL6.238 | | TGAAAATACAAATGACCATGCAAGTAATTCCGCAGGGAGAGAGCGGATATGAACAAACAGAAGAA |
| | | | ATCAGATGGGATAGTGCTGGCGGGAAGTCA (SEQ ID NO: 113) |
| 108 | HGL6.239 | | AATCGAAAGGAATGTCATCGAATGGAATGGACTCAAATGGAATAGAATCGGATGGAATGGCATCG |
| | | | AATGGAATGGAATGGAATTGGATGGAC (SEQ ID NO: 114) |
| 109 | HGL6.241 | | AACATGAACAGTGGAACAATCAGTGAACCAATACAAGGGTTAAATAAGCTAGCAATTAAAAGCTGT |
| | | | ATCACTGGTCTAAAGATAGAAGATCAAGTAGAAAATCAGCGCAAGAGGAAAGATATACGAAAACTA |
| | | | ATGGCC (SEQ ID NO: 115) |
| 110 | HGL6.243 | | CGAATGGAATCATTATGGAATGGAATGAAATGGAATAATCAAATGGAATTGAATGGAATCATCGAA |
| | | | TGGAATCGAACAAAATCCTCTTTGAATGGAATAAGATGGAATCACCAAATGGAATTG (SEQ ID NO: |
| | | | 116) |
| 111 | HGL6.246 | | AAACGGAATCAAACGGAATTATCGAATGGAATCGAAGAGAATCATCGAACGGACTCGAATGGAATC |
| | | | ATCTAATGGAATGGAATGGAAGAATCCATGGACT (SEQ ID NO: 117) |
| 112 | HGL6.247 | | GCTAGTTCAACATATGCAAATCAATAAACGTAATCCATCACATAAACAGAACCAATGACAAAAACCA |
| | | | CGATTATCTCAATAGATGCAGAAAAGGCC (SEQ ID NO: 118) |
| 113 | HGL6.256 | | ACCAATCAAGAAAACAATGCAACCCACAGAGAATGGACAAAAGCAAGGCAGGACAATGGCT (SEQ |
| | | | ID NO: 119) |
| 114 | HGL6.26 | | ATCGAATGGAATCAACATCAGACGGAAAAAAACGGAATTATCAAATGGAATCGAAGAGAATCATCG |
| | | | AATGGACC (SEQ ID NO: 120) |
| 115 | HGL6.260 | | ATGGAATCAACATCAAACGGAAAAAAAAACGGAATTATCGAATGGAATCGAAGAGAATCATCGAAT |
| | | | GGACCAGAATGGAATCATCTAATGGAATGGAATGG (SEQ ID NO: 121) |
| 116 | HGL6.261 | HGL6.1088 | AATGGAATCATCATCGAATGGAATCGAATGGAATCATGGAATGGAATCAAATGGAATCAAATGGAA |
| | | | TCGAATGGAATGGAATGGAATG (SEQ ID NO: 122) |
| 117 | HGL6.262 | | AACGGAATCAAACGGAATTACCGAATGGAATCGAATAGAATCATCGAACGGACTCGAATGGAATCA |
| | | | TCTAATGGAATGGAATGGAAG (SEQ ID NO: 123) |
| 118 | HGL6.263 | | AAACGGAATCAAACGGAATTATCGAATGGAATCGAAAAGAATCATCGAACGGACTCGAATGGAATC |
| | | | ATCTAATGGAATGGAATGGAAGAATCCATGG (SEQ ID NO: 124) |
| 119 | HGL6.266 | | AGCAACTTCAGCAAAGTCTCAGGATACAAAATCAATGTACAAAAATCACAAGCATTCTTATACACCA |
| | | | ATAACAGACAAACAGAGAGCC (SEQ ID NO: 125) |
| 120 | HGL6.267 | | GAATGATACGGANTANNNNGNAATGGAACGAAATGAAATGGAATGGAATGGAATGGAATGGAAT |
| | | | GGAATGG (SEQ ID NO: 126) |
| 121 | HGL6.268 | | AATGGACTCGAATGGATTAATCATTGAACGGAATCGAATGGAATCATCATCGGATGGTAATGAATG |
| | | | GAATCATCATCGAATGGAATCGG (SEQ ID NO: 127) |
| 122 | HGL6.271 | | GAATGGAATCGAAAGGAATGTCATCGAATGGAATGGAATGGAACGGAATGGAATCGAATGGAATG |
| | | | GACTCGAATGGAATAGAATCGAATGCAATGGCATCG (SEQ ID NO: 128) |
| 123 | HGL6.274 | HGL6.466, | GAATAGAATAGAATGGAATCATCGAATGGAATCGAATGGAATCATCATGATATGGAATTGAGTGGA |
| | | HGL6.883 | ATC (SEQ ID NO: 129) |
| 124 | HGL6.276 | | TAAGCCGATAAGCAACTTCAGCAAAGTCTCAGGAGACAAAATCAATGTGCAAAAAATCACAAGCAT |
| | | | TCTTATACACTAATAACAGACAAACAGAGAGCCAAATCATG (SEQ ID NO: 130) |
| 125 | HGL6.277 | | AGCAACTTCAGCAAAGTCTCAGGATACAAAATCAATGTGCAAAAATCAAAAGCATTCTTATGCACCA |
| | | | ATAACAGACACAGAGCCAAAT (SEQ ID NO: 131) |
| 126 | HGL6. 278 | | AGGAAAGTTTTCAATATGAGAAAGATACAAACCAACAGAATAAGCAAACTGGATAAACAGAAAATA |
| | | | CAGAGAGAGCCAAGG (SEQ ID NO: 132) |
| 127 | HGL6. 280 | | AATGGAATGGAACGCAATTGAATGGAATGGAATGGAACGGAATCAACCTGAGTCAAATGGAATGG |
| | | | AATGGAATGGAATG (SEQ ID NO: 133) |
| 128 | HGL6. 289 | | AGGAAAATGCAAATCAGAACGACTATAACACACCATCTCAAACTCGTTAGGATGGCTATTATCAAAA |
| | | | AGTCAAGAGATAACAAATGTGGGCAAGGG (SEQ ID NO: 134) |
| 129 | HGL6. 290 | | GGAACGAAATCGAATGGAACGGAATAGAATAGACTCGAATGTCATGGATTGCTATGTAATTGATTG |
| | | | GAATGGAATGGAATCG (SEQ ID NO: 135) |
| 130 | HGL6. 291 | | GAATTGAAAGGAATGTATTGGAATAAAATGGAATCGAATAGGTTGAAATACCATAGGTTCGAATTG |
| | | | AATGGAATGGGAGGGACACCAATGGAATTG (SEQ ID NO: 136) |
| 131 | HGL6. 292 | | AACAAAACAAAAACCCAACTCAATAACAAGAAGACAAACAACCCAATTTAAAATGAGCAAAGAACT |
| | | | TGATAAACATGTCTCCAAAGAAGATACGGCCAAAGAGCAC (SEQ ID NO: 137) |
| 132 | HGL6. 295 | | ATGGTTAAAACTCAACAATGAAAACACAAACAGCGCAATTTAAAAATGGGCAAAATGACAGGCCAG |
| | | | ACCCAGTGGCTCATGCG (SEQ ID NO: 138) |
| 133 | HGL6.300 | | AAGCAACTTCAGCAAAGTCTCGGGATACAAAATCAATGTGCAAAAATCACAAGCATTCTTATACACC |
| | | | ACTAACAGACAAATGGAGAGTC (SEQ ID NO: 139) |
| 134 | HGL6.302 | | GAATGGAATCAACATCAAACGGAAAAAAACGGAATTATCGAATGGAATCGAAGAGAATCATCGAAT |
| | | | GGACCAGAATGGAATCATCTAATGGAATGGAATGGAATAATCCATGG (SEQ ID NO: 140) |
| 135 | HGL6.305 | | TAGAAGGAATTTGATACATGCTCAGAAATACAGGCAAAGGAAGTAGGTGCCTGCCAGTGAACACAG |
| | | | GGGAACTATGGCTCCTA (SEQ ID NO: 141) |
| 136 | HGL6.310 | | GGAATCGAATGGAATCAACATCAAACGGAAAAAAACGGAATTATCGAATGGAATCGAAGAGAATC |
| | | | ATCGAATGGACC (SEQ ID NO: 142) |
| 137 | HGL6.311 | | AACTAAGACAACAGATTGATTTACACTACTATTTTCACACAGCCAAAAATATCACTATGGCAATCGTC |
| | | | AAAAGGTCAATTCAAAGATGGGACAGT (SEQ ID NO: 143) |
| 138 | HGL6.315 | | AAAAGCAATTGGACTGATTTTAAATATACGTGGCAACAAGGATAAACTGCTAATGATGGGTTTGCAA |
| | | | ATACAGATCG (SEQ ID NO: 144) |
| 139 | HGL6.317 | HGL6.1189 | AATGGAATCAACATCGAACGGAAAAAAACGGAATTATCGAATGGAATCGAAGAGAATCATCGAATG |
| | | | GACC (SEQ ID NO: 145) |
| 140 | HGL6.319 | | TGCAAGATAACACATTTTAGTTGACACCATTGAAAACAGTTTTAACCAAGAATATTAGAACCAATGA |
| | | | AGCAGAGAAATCAAAAGGGTGGATGGAACTGCCAAAGGATG (SEQ ID NO: 146) |
| 141 | HGL6.321 | | TAGAACAGAATTGAATGGAATGGCATCAAATGGAATGGAAACGAAAGGAATGGAATTGAATGGAC |
| | | | TCAAATGTTATGGAATCAAAGGGAATGGACTC (SEQ ID NO: 147) |
| 142 | HGL6.323 | | AAGAGAATCATCGAATGGAATCGAATGGAATCAACATCAAACGGAAAAAAACGGAATTATCGAATG |
| | | | GAATCGAAGAGAATCATCGAATGGACC (SEQ ID NO: 148) |
| 143 | HGL6.324 | HGL6.431, | ATCAACATCAAACGGAAAAAAACGGAATTATCGAATGGAATCGAAGAGAATCATCGAATGGACC |
| | | HGL6.1071 | (SEQ ID NO: 149) |
| 144 | HGL6.326 | | GAATCAACATCAAACGGAAAAAAACCGAATTATCGAATGGAATCGAAGAGAATCATCGAATGGACC |
| | | | (SEQ ID NO: 150) |
| 145 | HGL6.327 | | ATCAACATCAAACGGAATCAAACGGAATTATCGAATGGAATCGAAGAGAATCATCAAATGGACTCG |
| | | | AATGGAATCATCTAATGGAATGGAATGGAAGAATCCATGG (SEQ ID NO: 151) |
| 146 | HGL6.330 | HGL6.1005 | AAACAGTTCAAAAATTATTGCAACAAAATGAGAGAGATGAGTTTATCTTGCAAACTAATGGATGGTA |
| | | | GCAGTGACAGTGGCAAAACGTGGTTTGATTCT (SEQ ID NO: 152) |
| 147 | HGL6.334 | | ATCGAATGGAATCATTGAATGGAAAGGAATGGAATCATCATGGAATGGAAACGAATGGAATCACTG |
| | | | AATGGACTCGAATGGGATCATCA (SEQ ID NO: 153) |
| 148 | HGL6.335 | | ATTCAGCCTTTAAAAAAAGAAGACAGTCCTGTCATTTGTGACAATATGAATGAAACAGACATCACAT |
| | | | TAAATGAAATGAGCCAGGCGCAG (SEQ ID NO: 154) |
| 149 | HGL6.336 | | AGGAGAATAGCAGTAGAATGACAAAATTAGATTTTCACATGAAACTTGATGACAGTGTAGGAAATG |
| | | | GACTGAAAGGACAAGAC (SEQ ID NO: 155) |
| 150 | HGL6.337 | HGL6.1095, | AACCCACAAAGACAACAGAAGAAAAGACAACAGTAGACAAGGATGTCAACCACATTTTGGAAGAG |
| | | HGL6.1367 | ACAAGTAATCAAACACATGGCA (SEQ ID NO: 156) |
| 151 | HGL6.338 | | GAAAATGAACAATATGAACAAACAAACAAAATTACTACCCTTACGAAAGTACGTGCATTCTAGTATG |
| | | | GTGACAAAAAGGAAAG (SEQ ID NO: 157) |
| 152 | HGL6.339 | | AACATCAAACGGAATCAAACGGAATTATCGAATGGAATCGAAGAGAATCATCGAACGGACTCGAAT |
| | | | GGAATCATCTAATGGAATGGAATGGAAGAATCCATGGACTCGAATGCAATCATCATCGAATGAAAT |
| | | | CGAATGGAATCATCGAATGGACTCG (SEQ ID NO: 158) |
| 153 | HGL6.340 | | ACCAACATAAGACAAAGAAACATCCAGCAGCTGCCTATGGCAAAAGATTACAATGTGTCAAACAAG |
| | | | AGGGCAATG (SEQ ID NO: 159) |
| 154 | HGL6.342 | | ATGGAATTCAATGGAATGGACATGANTGNAATGNACTTCAATGGAATGGNATCNAATGGAATGNA |
| | | | ATTCANT (SEQ ID NO: 160) |
| 155 | HGL6.343 | | TATGACTTTCACAAATTACAGAAAAAGACACCCATTTGACAAGGGAACTGAAGGTGGTGAAGACAT |
| | | | ACTGGCAGGCTAC (SEQ ID NO: 161) |
| 156 | HGL6.344 | | AATGGAAAGGAATCGAATGGAAGGGAATGAAATTGAATCAACAGGAATGGAAGGGAATAGAATA |
| | | | GACGGCAATGGAATGGACTCG (SEQ ID NO: 162) |
| 157 | HGL6.347 | | AGCCTATCAAAAAGTGGGCTAAGAATATGAATACACAATTCTCAAAAGAAGATATACAAATGGGCA |
| | | | ACAAACATATGAAAACATACTCAACATCACTAATGATCAGGGAAATG (SEQ ID NO: 163) |
| 158 | HGL6.352 | HGL6. 710 | AGCAACTTCAGCAAAGTATCAGGATACAAAATCAATGTACAAAAATCCCAAGCATTCTTATACACCA |
| | | | ACAACAGACAAACAGAGAGCC (SEQ ID NO: 164) |
| 159 | HGL6.353 | | AAAGACAATATACAAATGGCCAATAAGCACATGAAAAGACGCTCAACATCCTTAGTCGTTAAGGCA |
| | | | ATGCAAATCAAAACCACAATG (SEQ ID NO: 165) |
| 194 | HGL6.429 | | GCATAGAATCGAATGGAATTATCATTGAATGGAATCGAATGGAATCAACATCAAACGGAAAAAAAC |
| | | | GGAATTATCGAATGGAATCGAAGAGAATCATCGAATGGACCC (SEQ ID NO: 200) |
| 195 | HGL6.430 | | AATGGAATCGAANAGAATCATCGAACGGACTCGAATGGAATCATCTAATGGAATGGAATGGAATAA |
| | | | TCCATGGACCCGAATG (SEQ ID NO: 201) |
| 196 | HGL6.433 | | AAATGAATCGAATGGAATTGAATGGAATCAAATAGAACAAATGGAATCGAAATGAATCAAATGGAA |
| | | | TCGAATCGAATGGAATTGAATGGCATGGAATTG (SEQ ID NO: 202) |
| 197 | HGL6.436 | | NTCACAATCACACAACACATTGCACATGNNNANNATGCACTCACAATACACACACAACACATACACA |
| | | | ACACACATGCAATACAACACAAAACGCAACACAACATATACACNACACACAGCACACANATGCC |
| | | | (SEQ ID NO: 203) |
| 198 | HGL6.442 | | GAATGGAATCAAATCGAATGAAATGGAATGGAATAGAAAGGAATGGAATGAAATGGAATGGAAAG |
| | | | GATTCGAAT (SEQ ID NO: 204) |
| 199 | HGL6.445 | | AAAGACTTAAACGTTAGACCTAAAACCATAAAAACCCTAGAGGAAAACCTAGGCATTACCATTCAGG |
| | | | ACTTAGGCATGGGCAAGGAC (SEQ ID NO: 205) |
| 200 | HGL6.446 | | GTTTACAGTCAAGTGTACAAACAGAATATAAGCAAACAAAAGAGAACATATACTTACAAACTATGCT |
| | | | AAGTGCCATGAAGGAAAAG (SEQ ID NO: 206) |
| 201 | HGL6.447 | | AAAGTCCAAAGATGAACAAAATATCCAGAAGGAAAACAAATGCACTTGGGGAGTGGGAAAGAAAA |
| | | | CCAAGACTGAGCAATGCGTCAAGCTCAGACGTGCCTCACTACG (SEQ ID NO: 207) |
| 202 | HGL6.448 | | AAACGGAATCAAACGGAATTATCGAATGGAGTCGAAAAGAATCATCGAACGGACTCGAATGGAATC |
| | | | ATCTAATGGAATGGAATGGAAGAATCCATGG (SEQ ID NO: 208) |
| 203 | HGL6.450 | HGL6.1296 | AATTGATTCGAAATTAATGGAATTGAATGGAATGCAATCAAATGGAATGGAATGTAATGCAATGGA |
| | | | ATGTAATAGAATGGAAAGCAATGGAATG (SEQ ID NO: 209) |
| 204 | HGL6.453 | | TACAGAACACATGACTCAACAACAGCAGAAAGCATATTCTTTTCAAATGCACATGAAACATTATCAT |
| | | | GATGGACCAAAT (SEQ ID NO: 210) |
| 205 | HGL6.454 | | TAAGACACATAGAAAACATAAAGCAAAATGGCAGATGTAAATGCAACCTATCAATCAAAACATTAC |
| | | | GAATGGCTT (SEQ ID NO: 211) |
| 206 | HGL6.456 | | GGAACAAAATGAAATCGAACGGTAGGAATCATACAGAACAGAAAGAAATGGAACGGAATGGAATG |
| | | | (SEQ ID NO: 212) |
| 207 | HGL6.457 | | AACGGAAAAAACGGAATTATCGAATGGAATCGAAGAGAATCATCGAATGGAATCGAATGGAGTCA |
| | | | TCG (SEQ ID NO: 213) |
| 208 | HGL6.459 | HGL6.806 | AACATACGAAAATCAATAAACGTAATCCAGCATATAAACAGAACCAAAGACAAAAACCACATGATTA |
| | | | TCTCAATAGATGCAGAAAAGGCCTTT (SEQ ID NO: 214) |
| 209 | HGL6.460 | HGL6.1163 | AATCGAACGGAATCAACATCAAACGGAAAAAAACGGAATTATCGAATGGAATCGAAGAGAATCATC |
| | | | GAATGGACC (SEQ ID NO: 215) |
| 210 | HGL6.461 | | AGAATGGAATGCAATAGAATGGAATGCAATGGAATGGAGTCATCCGTAATGGAATGGAAAGGAAT |
| | | | GCAATGGAATGGAATGGAATGG (SEQ ID NO: 216) |
| 211 | HGL6.462 | | GGAATAAAACGGACTCAATAGTAATGGATTGCAATGTAATTGATTCGATTTCGAATGGAATCGCATG |
| | | | GAATGTAATGGAATGGAATGGAATGGAAGGC (SEQ ID NO: 217) |
| 212 | HGL6.467 | | AGCAACTTCAGCAAAGTCTCAGGATACAAAATCAATGTACAAAAATCACAAGCATTCTTATACACCA |
| | | | ACAACAGACAAACAGAGAGCC (SEQ ID NO: 218) |
| 213 | HGL6.476 | | TAAGCAGAGAAAATATCAACACGAAAATAATGCAAGGAGAAAAATACAGAACAATCCAAAATGTG |
| | | | GCC (SEQ ID NO: 219) |
| 214 | HGL6.487 | | AATGGAATCAACATCAAACGGAAAAAAACGGAATTATCGTATGGAATCGAAAAGAATTATCGAATG |
| | | | GACC (SEQ ID NO: 220) |
| 215 | HGL6.489 | HGL6.587 | TCAAACGGAAAAAAACGGAATTATCGAATGGAATCGAAGAGAATCATCGAATGGACC (SEQ ID NO: |
| | | | 221) |
| 216 | HGL6.490 | | AACTTCAGCAAATTCTCAGGATACAAAATCAATGTGCAAAAACCACAAGCATTCCTATACACCAATA |
| | | | ATAGACAGTGAGCCAAAT (SEQ ID NO: 222) |
| 217 | HGL6.494 | HGL6.1131 | ACATCAAACGGAATCAAACGGAATTATCGAATGGAATCGAAAAGAATCATCGAACGGACTCGAATG |
| | | | GAATCATCTAATGGAATGGAATGGAAGAATCCATGGACTCGAATG (SEQ ID NO: 223) |
| 218 | HGL6.497 | | AATGGAATCGAATGCAATCATCGAACGGAATCGAATGGCATCACCGAATGGAATGGAATGGAATG |
| | | | GAATGGAATGG (SEQ ID NO: 224) |
| 219 | HGL6.499 | | AATCCAGCATATAAACAGAACCAAAGACAAAAACCACATGATTATCTCAATAGATGCAGAAAAGGC |
| | | | C (SEQ ID NO: 225) |
| 220 | HGL6.500 | | TGACTAAACAGAGTTGAACAAGAACAAAAAGCAAATTTGCAGAAATGAAATACATACTAATTGAAA |
| | | | GTCCATGGACAGGCTCAACAGATGATATAGATACAGCTAAAGAGATAATTAGTGAAATGGATCAG |
| | | | (SEQ ID NO: 226) |
| 221 | HGL6.501 | | GATCATCAGAGAAACAGAGAAATGCAAATTAAAACCACAATGAGATACTATCTCCACACAAGTCAG |
| | | | AATGGCTAT (SEQ ID NO: 227) |
| 222 | HGL6.503 | | AGGATACAAAATCAATGTACAAAAATCACAAACATTCTTATACACCAACAACAGACAAACAGAGAGC |
| | | | CAAATCATGGGTG (SEQ ID NO: 228) |
| 223 | HGL6.505 | | TAAGCAACTTCAGCAAAGTCTCAGGATACAAAATCAATGTACAAAAATCACAAGCATTCTTATACAC |
| | | | CAACAACAGACAAACAAGAGTGCCAAATCATG (SEQ ID NO: 229) |
| 224 | HGL6.506 | | AGAATTGATTGAATCCAAGTGGAATTGAATGGAATGGAATGGATTAGAAAGGAATGGAATGGATT |
| | | | GGAATGGATTGGAATGGAAAGG (SEQ ID NO: 230) |
| 225 | HGL6.508 | | AATGGAATGCAATCGAATGGAATGGAATCGAACGGAATGGAATAAAATGGAAGAAAACTGGCAAG |
| | | | AAATGGAATCG (SEQ ID NO: 231) |
| 226 | HGL6.509 | | AACTGCATCAACTAACAGGCAAAATAACCAGCTAATATCATAATGACAGGATTAAATTCACAAATGA |
| | | | CAATATTAACCGTAAATGTAAATGGGCTA (SEQ ID NO: 232) |
| 227 | HGL6.510 | | TACAAAGAACTCAAACAAATCAGCAAGAACAAAAACAATCCCAACAAAATGTTGGACAAAGACATG |
| | | | AATAGACAATTCTCGAAAGAAGATGTACAAATGGCT (SEQ ID NO: 233) |
| 228 | HGL6.512 | | AGAGAAATGCAAATCAAAACCACAATGGAATACCATCTCACGCCAGTCAGAATGGCAATTATTAAAA |
| | | | AATCACAACAATTAATGATGGCAAGGCTGTGG (SEQ ID NO: 234) |
| 229 | HGL6.513 | | GTAAACAAACAATCAAGCAAGTAAGAACAGAAATAACAGCATTTGGCTTTTGAGTTAATGACAAGA |
| | | | ACACTCGGCATGGGAGCCTGGGTGAGCAAATCACAGATCTTC (SEQ ID NO: 235) |
| 230 | HGL6.514 | | GAATCAACCCGAGCGGAAAGGAATGGAATGGAATGGAATCAACACGAATGGAATGGAACGGAATG |
| | | | GAATGGGATGGGATGAAATGGAATGG (SEQ ID NO: 236) |
| 231 | HGL6.516 | | AGCAACTTCAGCAAAGTCTCAGGAGACAAAATCAATGTACAAAAATCACAAGCATTCTTATACACCA |
| | | | ATAACAGACAAACAGAGAGCC (SEQ ID NO: 237) |
| 232 | HGL6.520 | | AAGAAATGGAATCGAAGAGAATGGAAACAAACGGAATGGAATTGAATGGAATGGAATTGAATGGA |
| | | | ATGGGA (SEQ ID NO: 238) |
| 233 | HGL6.522 | | GACATGCAAACACAACACACAGCACACATGGAACATGCATCAGACATGCAAACACAACACACATAC |
| | | | CACACATGGCATATGCATCAGACGTGCCTCACTAC (SEQ ID NO: 239) |
| 234 | HGL6.528 | | TACAGATAAGAAAATTGAGACTCAAGAGTATTACATAAATTGTTTCAGCTACCACAGCAAAAAATGG |
| | | | TATGGTTGGGAATCAAGCTCAGGG (SEQ ID NO: 240) |
| 235 | HGL6.529 | | AAAGGAATGCACTCGAATGGAATGGACTTGAATGGAATGTCTCCGAATGGAACAGACTCGTATGAA |
| | | | ATGGAATCGAATGGAATGGAATCAAATGGAATTGATTTGAGTGAAATGGAATCAAATGGAATGGCA |
| | | | ACG (SEQ ID NO: 241) |
| 236 | HGL6.530 | | TGAAACAAATGATAATGAAAATACAACATACCAAACATACGAGATACAGTAAAAGCAGTACTAAGA |
| | | | TGCAAGTATATATTGCTACAAGTGCCTAC (SEQ ID NO: 242) |
| 237 | HGL6.531 | | GGAACAAAATGAAATCGAACGGTAGGAATCGTACAGAACGGAAAGAAATGGAACGGAATGGAAT |
| | | | GCACTCGAATGGAAAGGAGTCCAAT (SEQ ID NO: 243) |
| 238 | HGL6.532 | | AAATTGATTGAAATCATCATAAAATGGAATCGAAGGGAATCAACATCAAATGGAATCAAATGGAAT |
| | | | CATTGAACGGAATTGAATGGAATCGTCAT (SEQ ID NO: 244) |
| 239 | HGL6.533 | | AGAAAGGATTCGAATGGAATGAAAAAGAATTGAATGGAATAGAACAGAATGGAATCAAATCGAAT |
| | | | GAAATGGAATGGAATAGAAAGGAATGGAATG (SEQ ID NO: 245) |
| 240 | HGL6.534 | | AGAATGGAAAGCAATAGAATGGAACGCACTGGATTCGAGTGCAATGGAATCAATTGGAATGGAAT |
| | | | CGAATGGAATGGATTGGCA (SEQ ID NO: 246) |
| 241 | HGL6.535 | | AACACCAAACGGAAAAAAACGGAATTATCGAATGGAATCGAAGAGAATCTTCGAACGGACCCGAAT |
| | | | GGGATCATCTAATGGAATGGAATGGAATAATCCATGG (SEQ ID NO: 247) |
| 242 | HGL6.536 | | AATGGAGACTAATGTAATAGAATCAAATGGAATGGCATCGAATGGAATGGACTGGAATGGAATGT |
| | | | GCATGAATGGAATGGAATCGAATGGATTG (SEQ ID NO: 248) |
| 243 | HGL6.539 | | TGGGATATGGGTGAAAGAACAAGTTTGCAGAAAAGATACAGTGAATTATGGACCATGAGTTCGGG |
| | | | AAAGAAGGGTAGGACTGCG (SEQ ID NO: 249) |
| 244 | HGL6.540 | | AAATCGAATGGAACGCAATAGAATAGACTCGAATGTAATGGATTGCTATGTAATTGATTCGAATGG |
| | | | AATGGAATCGACTGGAATGCAATCCAATGGAATGGAATGCAATGCAATGGAATGGAATCGAACGG |
| | | | AATGCAGTGGAAGGGAATGG (SEQ ID NO: 250) |
| 245 | HGL6.541 | | AATCAACAAGGAACTGAAACAAGTAAACAAGAAAACAAATAACACCATAAAACATGGGCAAAGGA |
| | | | CATAAACAGACATTTTTCAAAAAAGACATACAAATGGCCGAG (SEQ ID NO: 251) |
| 246 | HGL6.542 | | AATGGAATCAACATCAAACGGAAAAAAACGGAATTATCGAATGGAATCGAAGAGAATCATCGAATG |
| | | | GACCCAGGCTGGTCTTGAACTCC (SEQ ID NO: 252) |
| 247 | HGL6.545 | | ATTGAATGGGCTAGAATGGAATCATCTTTGAACGGAATCAAAGGGAATCATCATCGAATGGAATCG |
| | | | AATGGAAATGTCAACG (SEQ ID NO: 253) |
| 248 | HGL6.547 | | AATGGACTCGAATGGAATCAACATCAAATGGAATCAAGCGGAATTATCGAATGAAATCGAAGAGAA |
| | | | TCATCGAATGGACTCGAAAGGAATCATCTAATGGAATGGAATGGAATAATCCATGGACTCGAATGC |
| | | | AATCATCATCG (SEQ ID NO: 254) |
| 249 | HGL6.549 | | ACAGACAGAGATTTAAAACAATAAACAAGCAGTAAGCAAACACAGATAACAAAATGACATGATCCA |
| | | | ACAAATACTCAGAAGGAGACTTAGAAATGAATTGAGGGTC (SEQ ID NO: 255) |
| 250 | HGL6.553 | | AATGTAATCCAGCATATAAACAGAGCCAAAGACAAAAACCACATGATTATCTCAATAGATGCAGAAA |
| | | | AAGCCTTTGACAAAATTCAACAACCCTTCATGCTAAAAACTCTCAATAAATTAGGTATTGATGGGAC |
| | | | G (SEQ ID NO: 256) |
| 251 | HGL6.555 | | AAACGGAAAAAAACGGAATTATTGAATGGAATCGAAGAGAATCTTCGAACGGACCCGAATGGAATC |
| | | | ATCTAATGGAATGGAATGGAATAATCCATGG (SEQ ID NO: 257) |
| 252 | HGL6.557 | HGL6.1238 | GCTCAAGGAAATAAAATAGGACACAAAGAAATGGAAAAACATTCCATACTCATGGATAGAAAGAAT |
| | | | CAATATCATGAAATGGCC (SEQ ID NO: 258) |
| 253 | HGL6.560 | | ACTCGAGTGGAATTGACTGTAACAAAATGGAAAGTAACGGATTGGAATCGAATGGAACGGAATGG |
| | | | AATGGAATGGACAT (SEQ ID NO: 259) |
| 254 | HGL6.561 | | TACAAACTTTAAAAAATGATCAACAGATACACAGTTAGCAAGAAAGAATTGAGGGCAAAGAATATG |
| | | | CCAGACAAACTCAAGAGGAAGATGATGGTAGAGATAGGTCACATTGGAGTGTCA (SEQ ID NO: 260) |
| 255 | HGL6.562 | HGL6.154, | GGAATCGAATGGAATCAATATCAAACGGAAAAAAACGGAATTATCGAATGGAATCGAAGAGAATC |
| | | HGL6.114 | ATCGAATGGACC (SEQ ID NO: 261) |
| 256 | HGL6.564 | | AACGGAATCAAACGGAATTATCGAATGGAATCGAAAAGAATCATCGAACGGACTCGAATGGAATCA |
| | | | TCTAATGTAATGGAATGGAAGAATCCATGGACTCGAATG (SEQ ID NO: 262) |
| 257 | HGL6.565 | | GGAAATAACAGAGAACACAAACAAATGGGAAAACATTCCATGTTCATGGATAGGAAGAATCAATAT |
| | | | TGTGAAAATGGCCATACT (SEQ ID NO: 263) |
| 258 | HGL6.570 | | AACGGAAAAAAACGGAATTATCGAATGGAATCGAAGAGAATCATCGAATGGACCAGAATGGAATC |
| | | | ATCTAATGGAATGGAATGGAATAATCCATGGACTCGAATG (SEQ ID NO: 264) |
| 259 | HGL6.581 | | CAACATCAAACGGAAAAAAACGGAATTATGGAATGGAATCGAAGAGAATCATCGAATGGACCCGA |
| | | | ATGGAATCATCTGAAATATAATAGACTCGAAAGGAATG (SEQ ID NO: 265) |
| 260 | HGL6.589 | | ATGGAATCGAATGGAATGGACTGGAATGGAATGGATTCGAATGGAATCGAATGGAACAATATGGA |
| | | | ATGGTACCAAATG (SEQ ID NO: 266) |
| 261 | HGL6.595 | HGL6.1293 | GAATGGAATCAACATCAAACGGAAAAAAACGGAATTATCGAATGGAATCGAAGAGAATCATCGAAT |
| | | | GGACC (SEQ ID NO: 267) |
| 262 | HGL6.606 | | AAGGAATTTAAGCAAATCAACAAGCAAAACCAAAATAATCCCATTAAAAAGTGGGTAAAGGACATG |
| | | | AATACACACTTGTCAATAGAGGACATTCAAGTGGCCAAC (SEQ ID NO: 268) |
| 263 | HGL6.608 | | AAATGGACTCGAATGGAATCATCATAGAATGGAATCGAATGCAATGGAATGGAATCTTCCGGAATG |
| | | | GAATGGAATGGAATGGAATGGAG (SEQ ID NO: 269) |
| 264 | HGL6. 609 | | GAATCANCNNNNNNNGGAATCGAATGGAATCAACATCAAATGGAATCAAATGGAATCATTGAACG |
| | | | GAATTGAATGGAATCGTCAT (SEQ ID NO: 270) |
| 265 | HGL6.610 | | AGCAACTTCAGCAAAGTCTCAGGATACAAAATCAATGTACAAAAATCACAAGCATTCTTATACACCA |
| | | | ATAACAGACAAACAGAGAGCCAAAA (SEQ ID NO: 271) |
| 266 | HGL6.611 | | TATGCAAATCAATAAACATAATCCATCACATAAACAGAAACAAAGACAAAATGACATGATTATCTCA |
| | | | ATAGATGCAGAAAAGGCC (SEQ ID NO: 272) |
| 267 | HGL6.615 | | AGTAAATCACCATAAAGAAGGTAAGAGTTCATTCACAAAAACAACAAACTGAAGAATCAGGCCATA |
| | | | GTA (SEQ ID NO: 273) |
| 268 | HGL6.617 | | AGAAACAGAAAACAGTCAAACCAATGGGCAATCCATATCAGATGCAGTATTATGAACAGAAGTGTA |
| | | | AAGAATGCACCAGGCACAATGGC (SEQ ID NO: 274) |
| 269 | HGL6.619 | | AGGAAAAACAACAACAACAACAGGAAAACAACCTCAGTATGAAGACAAGTACATTGATTTATTCAA |
| | | | CATTTACTGATCACTTTTCAGGTGGTAGGCAG (SEQ ID NO: 275) |
| 270 | HGL6.623 | | GATTGGAACGAAATCGAATGGAACGGAATAGAATAGACTCGAATGTAATGGATTGCTATGTAATTG |
| | | | ATTCGAATGGAATGGAATCGAATGGAATGCAATCCAATGGAATGGAATGCAATGCAATGGAATGG |
| | | | (SEQ ID NO: 276) |
| 271 | HGL6.624 | | AACATATGGAAAAAAACTCAACATCACTGATCATTAGAGAAATGCAAATCAAAACCACAATGAGATA |
| | | | CCATCTCACGCCAGTCAGAATGGCG (SEQ ID NO: 277) |
| 272 | HGL6.625 | | ATGGAATGGAATAATCAACGTACTCGAATGCAATCATCATCGTATAGAATCGAATGGAATCATCGAA |
| | | | TGGACTCGAATGGAATAATCATTGAACGGAGTCGAATGGAATCATCATCGGATGGAAAC (SEQ ID |
| | | | NO: 278) |
| 273 | HGL6.627 | | AAANAANTCNAATGGAATCNNTGNCGAATGGAATGGAATGGAATCGAANAATTGAATTGNNNAN |
| | | | AATCNNANGNAANCNTTGNATGGGCTCAAAT (SEQ ID NO: 279) |
| 274 | HGL6.629 | | AGAAAAGATAACTCGATTAACAAATGAACAAACACCTGAATACACAAGTCTCAAAAGAAGACATAA |
| | | | AAATGGCCAAC (SEQ ID NO: 280) |
| 275 | HGL6.632 | | ATGGAATCAACATCAAACGGAATCACACGGAATTATCGAATGGAATCGAAAAGAATCATCGAACGG |
| | | | ACTCGAATGGAATCATCTAATGGAATGGAATGGAAG (SEQ ID NO: 281) |
| 276 | HGL6. 633 | HGL6.1135 | AATGGAATCAACATCAAACGGAATCAAGCGAAATTATCGAATGGAATCGAAGAGAATCATCGAATG |
| | | | GACTCGAATGGAATCATCTAATGGAATGGAATGGGAT (SEQ ID NO: 282) |
| 277 | HGL6.634 | | AAACACAGTACAAATACTAATTCAAATCAAACTTACTCAAAGTCATAATCAAACATGCCAGACGGGC |
| | | | TGAGGGGCAGCATTA (SEQ ID NO: 283) |
| 278 | HGL6.638 | | AACCACTGCTCAAGGAAATAAGAGAGAACACAAACAAATGAAAAAACATTCCATGCTCATGGATAG |
| | | | GAAGAATCAG (SEQ ID NO: 284) |
| 279 | HGL6.641 | | GGAATCGAGTGGAATCATCGAAAGAAATCGAATGGAATCATTGTCGAATGGAATGGAATGGAATC |
| | | | AAAGAATGGAATCGAAGGGAATCATTGGATGGGCT (SEQ ID NO: 285) |
| 280 | HGL6.642 | | AAAGAAAGACAGAGAACAAACGTAATTCAAGATGACTGTTTACATATCCAAGAACATTAGATGGTC |
| | | | AAAGACTTTAAGAAGGAATACATTCAAAGGCAAAAAGTCACTTACTGATTTTGGTGGAGTTTGCCAC |
| | | | ATGGAC (SEQ ID NO: 286) |
| 281 | HGL6.645 | | AAGATAGAGTTGAAACAGTGGACAATTAAAGAGTAATTTGGAAGAATGGTGAAATTACAGCCATGC |
| | | | TTTGAATCAGGCGGGTTCACTGGC (SEQ ID NO: 287) |
| 282 | HGL6.646 | | AAGAGTATCAACAGTAAATTACATTAGCAGAAGAATCAACAAACATGAAAATAGAAATTATGGTAG |
| | | | CCAAAGAACAG (SEQ ID NO: 288) |
| 283 | HGL6.647 | | GAAAGGAATCATCATTGAATGCAATCACATGGAATCATCACAGAATGGAATCGTACGGAATCATCAT |
| | | | CGAATGGAATTGAATGGAATCATCAATTGGACTCGAATGGAATCATCAAATGGAATCGATTGGAAG |
| | | | TGTCAAATGGACTCG (SEQ ID NO: 289) |
| 284 | HGL6.651 | | CAGCGCACCACAGCACACACAGTATACACATGACCCACAATACACACAACACACAACACATTCACAC |
| | | | ACCAC (SEQ ID NO: 290) |
| 285 | HGL6.655 | | GCAAACAGAATTCAACACTACATTAGAACGATCATTCATCACGACCTAGTAGGATGTTTTTCCTGGG |
| | | | ATGCAAGGATGGTTCAACAT (SEQ ID NO: 291) |
| 286 | HGL6.656 | | CAATCAAAACAGCAATGAGATACCATTTTACACCAATCAAAATGGCTACTAAAAAGTCAAAAGCAAA |
| | | | TGCC (SEQ ID NO: 292) |
| 287 | HGL6.658 | HGL6.830 | AGAACCATATTGAAGAGACAGAGTGATATATAAAACTGCTAACTCAAGCAGCACAAGAATTAAATG |
| | | | AATACCAAGAAAATACTTGGCCAG (SEQ ID NO: 293) |
| 288 | HGL6. 660 | | TGGAATAGAATGGAATCAATGTTAAGTGGAATCGAGTGGAATCATCGAAAGAAATCGAATGGAATC |
| | | | ATTGTCGAATGGTATGGAATGGAATCA (SEQ ID NO: 294) |
| 289 | HGL6.661 | | AATGGAATGGAATCATCGCATAGAATNGAATGGAATTATCATCGAATTGAATCGAATGGTATCAAC |
| | | | ATCAAACGGAAAAAAACGGAAATATCGAANGGAATCGAAGAGAATCATCGAACGGACC (SEQ ID |
| | | | NO: 295) |
| 290 | HGL6.662 | | ACATACGCAAATCAATAAACATAATCCATCACATAAACAGAACCAAAGACAAAAATCACATGATTAT |
| | | | CTCAATAGATGCAGAAAAGGCCTTCGAC (SEQ ID NO: 296) |
| 291 | HGL6.663 | | AAAAAATGTTCAACATCACTAGTCAGCAGAGAAATGCAAATCAAAATCACAATGAGATAACTTCTCA |
| | | | CACCAGACAGCATGGC (SEQ ID NO: 297) |
| 292 | HGL6.668 | | GAAAAACAAAACAAAACAAACAAACAAACAATCAAAAAAGTGGTAGCAGAAACCAGAAAGTCCAT |
| | | | GTATATAGCTAATTGGCCTGGTTGT (SEQ ID NO: 298) |
| 293 | HGL6.671 | | AACAGCAATGACAATGATCAGTAACAACAAGACTTTTAACTTTGAAAAAATCAGGACC (SEQ ID NO: |
| | | | 299) |
| 294 | HGL6.672 | | AAGAGCCTGAATAGCTAAAGTGATCATAAGCAAAAAGAACAAAGTCGGAAGCATCACATTACCTGA |
| | | | CTTCAAACTATACTCAAAGGCTATG (SEQ ID NO: 300) |
| 295 | HGL6.675 | | AAAAGGAAATACAAGACAACAAACACAGAAACACAACCATCGGGCATCATGAAACCTCGTGAAGAT |
| | | | AATCATCAGGGT (SEQ ID NO: 301) |
| 296 | HGL6.677 | | AAGCAAAGAAAGAATGAAGCAGCAAAAGAACGAAAGCAGGAATTTATTGAAAACCAAAGTACACT |
| | | | CCACAGTATGGGAGCGGACCCGAGCA (SEQ ID NO: 302) |
| 297 | HGL6.679 | | GCAAATGATTATAAGTGCTGTTATAGAAACATTCAAAGACCAGAAAAGGACCACAATGGCTGACCA |
| | | | C (SEQ ID NO: 303) |
| 298 | HGL6.681 | | AGAGCAGAAACAAATGGAATTGAAATGAAGACAACAATCAAAAGCATCAATGAAATGAAAAGTTG |
| | | | GGTTTTGGAAGAGAGAAACAAT (SEQ ID NO: 304) |
| 299 | HGL6.683 | | ACACAAACACACACACACACACACACACACACACACACACACACACACACACACACACACACACACA |
| | | | TAC (SEQ ID NO: 305) |
| 300 | HGL6.686 | | AACAAACAAATGAGATGATTTCAGATAGTGATAAACACTATAACATAATTAATTCGTGCCAATCAGA |
| | | | GCATAACAGTGGTGTGGTGGCTGTGGAACAGATAGCAGAC (SEQ ID NO: 306) |
| 301 | HGL6.688 | | AATGGAATCGAGTGGAATGGAAGGCAATGGAATAGAATGGAATGGAATCGAAAGGAACGGAATG |
| | | | GAATGGAATGGAATG (SEQ ID NO: 307) |
| 302 | HGL6.689 | | AGCAGTGCAAGAACAACATAACATACAAGTAAACAAACACATGGGGCCAGGTAATAAAAAGTCAG |
| | | | GCTCAAGAGGTCAG (SEQ ID NO: 308) |
| 303 | HGL6.690 | | AGAAATGGAATCGGAGAGAATGGAAACAAATGGAATGGAATTGAATGGAATGGAATTGAATGGAA |
| | | | TGGGAACG (SEQ ID NO: 309) |
| 304 | HGL6.694 | | GCACTAGTCAGATCAAGACAGAAAGTCAACGAACAAAGAACAGACTTAAACTACACTCTAGAACAA |
| | | | ATGGACCTA (SEQ ID NO: 310) |
| 305 | HGL6.704 | | AAGAGAACTGCAAAACACTGCTCAAAGAAATCAGAGATGACAAAAACACATGGAAAAACGTTTCAT |
| | | | GCTCATGGATTGGAAGACTTA (SEQ ID NO: 311) |
| 306 | HGL6.705 | | AATCAACACGAATAGAATGGAACGGAATGGAATGGAATGGAATGGAATGGAATGGAGTGGAATG |
| | | | GAACAGAATGGAGTGGAAT (SEQ ID NO: 312) |
| 307 | HGL6.707 | | AACATCAAACGAAATCAAACGGAATTATCAAATTGAATCGAAGAGAATCATCGAATTGCCACGAAT |
| | | | GCAACCATCTAATGGTATGGAATGGAATAATCCATGGACCCAGATG (SEQ ID NO: 313) |
| 308 | HGL6.714 | | CGGAATTATCATCGAATGTAATCGAATGGAATCAACATCAAACGGAAAAAAACGGAATTATCGAAT |
| | | | GGAATCGAAGAGAATCATCGAATGGACC (SEQ ID NO: 314) |
| 309 | HGL6.719 | | TGGACACACACGAACACACACCTACACACACGTGGACACACACGGACACATGGACACACACGAACA |
| | | | CATGGACACACACACGGGGACACACACAGACACACACAGAGACACACACGGACACATGG (SEQ ID |
| | | | NO: 315) |
| 310 | HGL6.720 | HGL6.1044 | AGCAACTTCAGCAAAGTCTCAGGATACAAAATCAATGTGCAAAAATCACAAGCATTCTTATACACCA |
| | | | ATAACAGACAAACAGAGAGCC (SEQ ID NO: 316) |
| 311 | HGL6.721 | HGL6.1020 | AAAATCAATATGAAAACAAACACAAGCAGACAAAGAAAATTGGGCAAAAGGTTTGAGCAGACACTT |
| | | | CACCAAAGAAGTACAAATGGCAAATCAGCA (SEQ ID NO: 317) |
| 312 | HGL6.724 | | ATCAAACGGAATCAAACGGAATTATCGAATGGAATCGAAGAGAATCATCGAATGGACTCGAATGGA |
| | | | ATCATCTAATGGAATGGAATGGAAGAATCCATGG (SEQ ID NO: 318) |
| 313 | HGL6.725 | | AACAGATTTAAACAAACCAACAAGCAAAAAACGAACAACTCCATTCAAACATGGACAAAAGACACG |
| | | | AACAGACACTTTTCAAAGAAGACATACATGTGGCC (SEQ ID NO: 319) |
| 314 | HGL6.726 | | AAATGGAATGGAATGCACTTGAAAGGAATAGACTGGAACAAAATGAAATCGAACGGTAGGAATCA |
| | | | TACAGAACAGAAAGAAATGGAACGGAATGGAATG (SEQ ID NO: 320) |
| 315 | HGL6.727 | | ACCACACACAAAATACACCACACACCACACACACACCACACACTATACACACACCACACACCACACAC |
| | | | (SEQ ID NO: 321) |
| 316 | HGL6.728 | | AAAGAAATAGAAGGGAGTTGAACAGAATCGAATGGAATCGAATCAAATGGAATCGAATGGCATCA |
| | | | AATGGAATCGAATGGAATGTGGTGAAGTGGATTGG (SEQ ID NO: 322) |
| 317 | HGL6.729 | | GGAATCATCATAAAATGGAATCGAATGGAATCATCATCAAATGGAATCAAATGGAATCATTGAACG |
| | | | GAATTGAATGGAATCGTCAT (SEQ ID NO: 323) |
| 318 | HGL6.730 | | TGGAATGGAATGGAATGAAATAAACACGAATAGAATGGAACGGAATGGAACGGAATGGAATGGA |
| | | | ATGGAATGGAAAG (SEQ ID NO: 324) |
| 319 | HGL6.731 | | AAGAATTGGACAAAACACACAAACAAAGCAAGGAAGGAATGAAAGGATTTGTTGAAAATGAAAGT |
| | | | ACACTCCACAGTGTGGGAGCAG (SEQ ID NO: 325) |
| 320 | HGL6.732 | | TAATCAGCACAATCAACTGTAGTCACAAAACAAATAGTAACGCAATGATAAAGAAACAGAGAACTA |
| | | | GTTCAAATAAACATGATAAGATGGGG (SEQ ID NO: 326) |
| 321 | HGL6.733 | | AAGCGGAATTATCAAATGGAATCGAAGAGAATGGAAACAAATGGAATGGAATTGAATGGAATGGA |
| | | | ATTGAATGGAATG (SEQ ID NO: 327) |
| 322 | HGL6.734 | | AAGCAACTTCAGCAAAGTCTCAGGACACAAAATCAATATGCGAAAATCACAAGCATTCCTATACACC |
| | | | AATAATAGACAAACAGAGAGCCAAATCATG (SEQ ID NO: 328) |
| 323 | HGL6.736 | | TTCACAGCAGCATTACGCACAATAGCCAGAAGGTGGGAACAGACAAAATGCCTTTTGATGGG (SEQ |
| | | | ID NO: 329) |
| 324 | HGL6.738 | | AGACCCTAATATCACAGTTAAACGAACTAGAGAAGGAAGAGCAAACAAATTCAAAAGCTAGCGGAA |
| | | | AGCAAGAAATAACTAAGACCAG (SEQ ID NO: 330) |
| 325 | HGL6.739 | | TAAAAGTGTGCTCAACATCATTGATCATCAGAGAAATGCAAATCAAAACTACAATGAGATATCATCT |
| | | | CATCCCAGTCAAAGTGGCT (SEQ ID NO: 331) |
| 326 | HGL6.740 | | ACTTGAATCGAATGGAAAGGAATTTAATGAACTTAAATCGAATGGAATATAATGGTATGGAATGGA |
| | | | CTCATGGAATGGAATGGAAAGGAATC (SEQ ID NO: 332) |
| 327 | HGL6.742 | | TGGAATCATCATCGAAAGCAAGCGAATGGAATCATCAAATGGAAACGAATGGAATCATCGAATGGA |
| | | | CTCGGATGGAATTGTTGAATGGACT (SEQ ID NO: 333) |
| 328 | HGL6.743 | | TGGAATCAACATCAAACGGAAAAAAACGGAATTATCGAATGGAATCGAAGAGAATCATCGAATGGA |
| | | | CC (SEQ ID NO: 334) |
| 329 | HGL6.745 | | TAAGTGAATTGAATAGAATCAATCTGAATGTAATGAAATGGAATGGAACGGAATGGAATGGAATG |
| | | | GAATGGAATGGAATGGAATGG (SEQ ID NO: 335) |
| 330 | HGL6.747 | | AGGAAAATTTAATCAGCAGGAATAGAAACACACTTGAGAAATCCATGTGGAATGAAAAGAGAATG |
| | | | GCTGAGCAGCAACAGATTGTCAAAAAGGAAATC (SEQ ID NO: 336) |
| 331 | HGL6.749 | HGL6.897 | AACATCAAACGGAAAAAAAACGGAATTATCGAATGGAATCGAAGAGAATCATCGAATGGACC (SEQ |
| | | | ID NO: 337) |
| 332 | HGL6.756 | | GAAAATGAACAATATGAACAAACAAACAAAATTACTACCCTTACGAAAGTACGTGCATTCTAGTATG |
| | | | GTGACAAAAAGGAAA (SEQ ID NO: 338) |
| 333 | HGL6.757 | | AGAAAACACACAGACAACAAAAAACACAGAACGACAATGACAAAATGGCCAAGC (SEQ ID NO: |
| | | | 339) |
| 334 | HGL6.758 | HGL6.1040 | AGCAACTTCAGCAAAGACTCAGGATACAAAATCAATGTGCAAAAATCACAAGCATTCTTATACACCA |
| | | | ATAACAGACAGAGAGCCAAAT (SEQ ID NO: 340) |
| 335 | HGL6.759 | | TGACATGCAAGAAATAAGGAAGTGCAAAAACAAACAAACAAACAACAACAACAACAACAACAACAA |
| | | | CAACAAAAAACAGTCCCAAAAGGATGGGCAG (SEQ ID NO: 341) |
| 336 | HGL6.760 | | TAATTGAGAATAAGCATTCCAGTGGAAAAAAAACTAAACAATTTGTTGTAAAACATCCTTAAAAGCA |
| | | | TCAGAAAGTTAATACAGCAATGAAGAATTACAGGACCAAATTAAGAATGGTATGGAAGCCTGTTA |
| | | | (SEQ ID NO: 342) |
| 337 | HGL6.762 | | TATCATCGAATGGAATCGAATGGAATCAACATCAAACGGAAAAAAACGGAATTATCGAATTGAATC |
| | | | GAAGAGAATCATCGAATGGACC (SEQ ID NO: 343) |
| 338 | HGL6.764 | | GAATGGAATCAAATAGAATGGAATCGAAACAAATGGAATGGAATGGAATGGGAGCTGAGATTGTG |
| | | | TCACTGCAC (SEQ ID NO: 344) |
| 339 | HGL6.765 | | AGCAAAACAAACACAATCTGTCGTTCATGGTACTACGACATACTGGGAGAGATATTCAAATGATCAC |
| | | | ACAAAACAACATG (SEQ ID NO: 345) |
| 340 | HGL6.766 | | AAGGATTCGAATGGAATGAAAAAGAATTGAATGGAATAGAACAGAATGGAATCAAATCGAATGAA |
| | | | ATGGAGTGGAATAGAAAGGAATGGAATG (SEQ ID NO: 346) |
| 341 | HGL6.768 | | AACGGAATCAAACGGAATTATCGAATGNNNTNNAAGAGAATCATCGAACGGACTCGAATGGAATC |
| | | | ATCTAATGGAATGGAATGGAAGAATCCATGGACTCGAATGCAATCATCATCGAATGGAATCGAACG |
| | | | GAATCATCGAATGGCC (SEQ ID NO: 347) |
| 342 | HGL6.771 | | AATCAACTAGATGTCAATGGAATGCAATGGAATAGAATGGAATGGAATTAACACGAATAGAATGGA |
| | | | ATGGAATGGAATGGAATGG (SEQ ID NO: 348) |
| 343 | HGL6.772 | | TGTAACACTGCAAACCATAAAAACCGTAGAAGAAAACCTAGACAATACTATTCAGGACATAGGCAT |
| | | | GGGCAAAGAC (SEQ ID NO: 349) |
| 344 | HGL6.773 | | AATGGACTCGAATGGAATAATCATTGAACGGAATCGAATGGAATCATCATCGGATGGAAATGAATG |
| | | | GAATCATCATCGCATGGAATCG (SEQ ID NO: 350) |
| 345 | HGL6.776 | | GAATGGAATGATACGGAATAGAATGGAATGGAACGAAATGGAATTGAAAGGAAAGGAATGGAAT |
| | | | GGAATGGAATGG (SEQ ID NO: 351) |
| 346 | HGL6.777 | | AAAAATGACCAGAGCAATAGAATGCATTGACCAGATAAAGACCTTCACGTATGTTGAACTAAAATGT |
| | | | GTGGTGCAGGTG (SEQ ID NO: 352) |
| 347 | HGL6.781 | | AATCATCATCGAATGGAATCGAATGGTATCATTGANTGNAATCGAATGGAATCATCATCANATGGA |
| | | | AATGAATGGAATCGTCAT (SEQ ID NO: 353) |
| 348 | HGL6.785 | | ACAAAATCAAACTAACCTCGATAAGAATGCAAGTGAATCAAAATGAGTTTCAAGGGGTTGTGGCTA |
| | | | GTACACGCTTTCTACAGCTG (SEQ ID NO: 354) |
| 349 | HGL6.787 | | GAATCAAATCAATGGAATCAAATCAAATGGAATGGAATGGAATTGTATGGAATGGAATGGCATGG |
| | | | (SEQ ID NO: 355) |
| 350 | HGL6.789 | | TAATGCAGTCCAATAGAATGGAATCGAATGGCATGGAATATAAAGAAATGGAATCGAAGAGAATG |
| | | | GGAACAAATGGAATGGAATTGAGTGGAATGGAATTGAATGGAATGGGAACGAATGGAGTG |
| | | | (SEQ ID NO: 356) |
| 351 | HGL6.792 | | TGAATAGACACACAGACCAATGGAACAGAATAGAGAACACAGAATAAATCTGCACACTTATAGCCA |
| | | | GCTGATTTTTGACAAATTTGCCAAG (SEQ ID NO: 357) |
| 352 | HGL6.797 | HGL6.810, | AACATCNNACGGAAAAAAACGGAATTATCGAATGGAATCGAAGAGAATCATCGAATGGACC (SEQ |
| | | HGL6.1172, | ID NO: 358) |
| | | HGL6.1223 | |
| 353 | HGL6.801 | | GCCAACAATCATATGAGAAAAAGCTCAACATCACTGATCATTTCAGGAATGCAAATCAAAACCACAA |
| | | | TGAGATACTATCACACATCAATCAGAATGGCT (SEQ ID NO: 359) |
| 354 | HGL6.802 | HGL6.118, | GAATCGAATGGAATCAACATCAAACGGAAAAAAACGGAATTATCGAATGGAATCGAAGAGAATCAT |
| | | HGL6.590, | CGAATGGACC (SEQ ID NO: 360) |
| | | HGL6.1051, | |
| | | HGL6.1170, | |
| | | HGL6.1248, | |
| | | HGL6.1372 | |
| 355 | HGL6.804 | | AATCAAATGGAATGAAATCGAATGGAATTGAATCGAATGGAATGCAATAGAATGTCTTCAAATGGA |
| | | | ATCGAATGGAAATTGGTGAAGTGGACGGGAGTG (SEQ ID NO: 361) |
| 356 | HGL6.805 | | TAACAGTACCAAAAAACAGTCATAATCTTCAAGAGCTTAAATTTAGCATGAAAGGAAGACATTCATC |
| | | | AAAGAATCACACAAAGGAATGTAAAATTAAATGGAGATTAGTGCCAGGAAAGAGC (SEQ ID NO: 362) |
| 357 | HGL6.808 | | TAATGGAATCAACATCAAACGGAAAAAAACGGAATTATCGAATGCAATCGAAGAGAATCATCGAAT |
| | | | GGACC (SEQ ID NO: 363) |
| 358 | HGL6.813 | | AGCAACTTCAGCAAAGTCTCAGCATACAAAATCAATGTGCAAAAATCACACGCATTCCTATACACCA |
| | | | ATAACAGACAAACAGAGAGCC (SEQ ID NO: 364) |
| 359 | HGL6.815 | | GAATCAAATGGAATGGACTGTAATGGAATGGATTCGAATGGAATCGAATGGAGTGGACTCAAATG |
| | | | GAATG (SEQ ID NO: 365) |
| 360 | HGL6.816 | | AACAAGTGGACGAAGGATATGAACAGACACTTCTCAAGACATTTATGCAGCCAACAGACACACGAA |
| | | | AAAATGCTCATCATCACTGGCCATCAG (SEQ ID NO: 366) |
| 361 | HGL6.819 | | AAACACACAAAGCAACAAAAGAACGAAGCAACAAAAGCATAGATTTATTGAAATGAAAGTACATTC |
| | | | TACAGAGTGGGGGCAGGCT (SEQ ID NO: 367) |
| 362 | HGL6.820 | | ATACAACTAAAGCAAATATAAGCAACTAAAGCAACAGTACAACTAAAGCAAAACAGAACAAGACTG |
| | | | CCAGGGCCTAGAAAAGCCAAGAAC (SEQ ID NO: 368) |
| 363 | HGL6.822 | | GCAATCGAATGGAATGGAATCGAACGGAATGGAATAAAATGGAAGAAAACTGGCAAGAAATGGAA |
| | | | TCG (SEQ ID NO: 369) |
| 364 | HGL6.825 | | AGCAGCCAACAAGCATATGAAATAATGCTCCACAACACTCATCATCAGAGAAATGCAAATCAAAACC |
| | | | AAAAT (SEQ ID NO: 370) |
| 365 | HGL6.826 | | TGGAACCGAACAAAGTCATCACCGAATGGAATTGAAATGAATCATAATCGAATGGAATCAAATGGC |
| | | | ATCTTCGAATTGACTCGAATGCAATCATCCACTGGGCTT (SEQ ID NO: 371) |
| 366 | HGL6.827 | HGL6.829 | AACGGAATCACGCGGAATTATCGAATGGAATCGAAGAGAATCATCGAATGGACTCGAATGGAATCA |
| | | | TCTAATGGAATGGAATGG (SEQ ID NO: 372) |
| 367 | HGL6.830 | | AGAACCATATTGAAGAGACAGAGTGATATATAAAACTGCTAACTCAAGCAGCACAAGAATTAAATG |
| | | | AATACCAAGAAAATACTTGGCCAG (SEQ ID NO: 373) |
| 368 | HGL6.831 | | AAAACAAACAACAACGACAAATCATGAGACCAGAGTTAAGAAACAATGAGACCAGGCTGGGTGTG |
| | | | GTG (SEQ ID NO: 374) |
| 369 | HGL6.833 | | AATCGAAAGGAATGCAATATTATTGAACAGAATCGAAAAGAATGGAATCAAATGGAATGGAACAG |
| | | | AGTGGAATGGACTGC (SEQ ID NO: 375) |
| 370 | HGL6.836 | | AAGGAATCGAATGGAAGTGAATGAAATTGAATCAACAGGAATGGAAGGGAATAGAATAGACTGTA |
| | | | ATGGAATGGACTCG (SEQ ID NO: 376) |
| 371 | HGL6.837 | | AATGGACTCGAATGAAATCATCATCAAACGGAATCGAATGGAATCATTGAATGGAATGGAATGGAA |
| | | | TCATCATGGAATGGAAACG (SEQ ID NO: 377) |
| 372 | HGL6.838 | | TTGACCAGAACACATTACACAATGCTAATCAACTGCAAAGGAGAATATGAACAGAGAGGAGGACAT |
| | | | GGATATTTTGTG (SEQ ID NO: 378) |
| 373 | HGL6.839 | | AACCCGAGTGCAATAGAATGGAATCGAATGGAATGGAATGGAATGGAATGGAATGGAATGGAGTC |
| | | | (SEQ ID NO: 379) |
| 374 | HGL6.843 | | AAGAGTATTGAAGTTGACATATCTAGACTGATCAAGAACAAAGACAAAAGGTACAGATTATCAAGA |
| | | | AAATGAGCGGGCAAAGCAAGATGGCC (SEQ ID NO: 380) |
| 375 | HGL6.847 | | GAATGGAATTGAAAGGAATGGAATGCAATGGAATGGAATGGGATGGAATGGAATGCAATGGAATC |
| | | | AACTCGATTGCAATG (SEQ ID NO: 381) |
| 376 | HGL6.849 | | GAAAAAAACGGAATTATCNAATTGAATCNAATANAATCATCNNNNNGACCANANTGGAATCATCT |
| | | | AATGNAATGNAATGGAATAATCCATGGACTCNAATG (SEQ ID NO: 382) |
| 377 | HGL6.850 | | GAAAAAAACGGAATTATCGAATTGAATCGAATAGAATCATCGAACGGACCAGAATGGAATCATCTA |
| | | | ATGGAATGGAATGGAATAATCCATGGACTCGAATG (SEQ ID NO: 383) |
| 378 | HGL6.853 | | AACCACTGCTTAAGGAAATAAGAGAGAACACAAACAAATGGAAAAACGTTCCATGCTCATGGATAG |
| | | | GAGAATCAATATCGTGAAAATGGCC (SEQ ID NO: 384) |
| 379 | HGL6.854 | | TATCGAATGGAATGGAAAGGAGTGGAGTAGACTCGAATAGAATGGACTGGAATGAAATAGATTCG |
| | | | AATGGAATGGAATGGAATGAAGTGGACTCG (SEQ ID NO: 385) |
| 380 | HGL6.855 | | GTATCAACATCAAACGGAAAAAAACGGAATTATCGAATGGAATCATCTAATGGAATGGAATGGAAT |
| | | | AATCCATGGACTCGAATG (SEQ ID NO: 386) |
| 381 | HGL6.856 | | TAAATGGAGACATCATTGAATACAATTGAATGGAATCATCACATGGAATCGAATGGAATCATCGTAA |
| | | | ATGCAATCAAGTGGAATCAT (SEQ ID NO: 387) |
| 382 | HGL6.857 | | GAATGGAATTGAAAGGTATCAACACCAAACGGAAAAAAAAACGGAATTATCGAATGGAATCGAAG |
| | | | AGAATCATCGAACGGACC (SEQ ID NO: 388) |
| 383 | HGL6.858 | | AGCAATTTCAGCAAAGTCTCAGGATACAAAATCAATGTACAAATTCACAAGCATTCTTATGGACCAA |
| | | | CAACAG (SEQ ID NO: 389) |
| 384 | HGL6.860 | | AACCAAATTAGACAAATTGGAAATCATTACACATAACAAAAGTAATAAACTGTCAGCCTCAGTAGTA |
| | | | TTCATTGTACATAAACTGGCC (SEQ ID NO: 390) |
| 385 | HGL6.861 | | TATTTTACCAGATTATTCAAGCAATATATAGACAGCTTAAAGCATACAAGAAGACATGTATAGATTTA |
| | | | CATGCAAACACTGCACCACTTTACATAAGGGACTTGAGCAC (SEQ ID NO: 391) |
| 386 | HGL6.863 | | GGAATCGAATGGCATCAACATCAAACGGAAAAAAACGGAATTATCGAATGGAATCGAATGGAATCA |
| | | | TC (SEQ ID NO: 392) |
| 387 | HGL6.864 | | AAACAAAACACAGAAATGCAAAGACAAAACATAAAACGCAGCCATAAAGGACATATTTTAGATAAC |
| | | | TGGGGAAATTTGTATGGGCTGTGT (SEQ ID NO: 393) |
| 388 | HGL6.866 | HGL6.867 | AGGAAAAGAAAGAAATAGAAAATGCGAAATGGTAAGAAAAAACAGCATAATAAACATTTGTATGG |
| | | | TGTTGATGGACAATGCATT (SEQ ID NO: 394) |
| 389 | HGL6.869 | | AATGGAATCAACATCAAACGGAATCAAACGGAATTATCGAATGGAATCGAAGAGAATCATCGAACG |
| | | | GACTCGAATGGAATCATCTAATGGAATGGAATGGAAG (SEQ ID NO: 395) |
| 390 | HGL6.872 | HGL6.1072, | AATCGAATGGAATCAGCATCAAACGGAAAAAAACGGAATTATCGAATGGAATCGAAGAGAATCATC |
| | | HGL6.1301 | GAATGGACC (SEQ ID NO: 396) |
| 391 | HGL6.877 | | AAAGAAATGGAATCGAAGAGAATGGAAACAAATGGAATGGAATTGAATGGAATGGAATTGAATGG |
| | | | AATGGGAACG (SEQ ID NO: 397) |
| 392 | HGL6.878 | | AGAAAGAATCAAGAGGAAATGCAAGAAATCCAAAACACTGTAACAGATATGATGAATAATGAGGT |
| | | | ATGCACTCATCAGCAGACTCGACAT (SEQ ID NO: 398) |
| 393 | HGL6.879 | | AAACGGAATTATNNANTGGANNNNAAGNNAATCATCGAACGGANNNNANNGGAATCATNTNNN |
| | | | NGAANGGAATGGAACAATCCATGGTNTNNNN (SEQ ID NO: 399) |
| 394 | HGL6.882 | HGL6.971 | AGCAACTTCAGCAAAGTTTCAGGATACAAAATCAATGTGCAAAAATCACAAGCATTCTTATACACCA |
| | | | ACAACAGACAAACAGAGAGCC (SEQ ID NO: 400) |
| 395 | HGL6.884 | | AGACAGTCAGACAATCACAAAGAAACAAGAATGAAAATGAATGAACAAAACCTTCAAGAAATATGG |
| | | | GATTATGAAGAGGCCAAATGT (SEQ ID NO: 401) |
| 396 | HGL6.885 | | ATCATAACGACANGANCAAATTCACACACAACAATNNNNACNNNAAANNCAAATGGGTTAAATNN |
| | | | TNCAATTAAAGGATGCAGACGGGCAAATTGGATA (SEQ ID NO: 402) |
| 397 | HGL6.891 | | ATCATAANGACAAGANCAAATTCACACACAACAATNNNNACNNNAAANNCAAATGGGTTNAATGN |
| | | | TNCAATTAAAGGATGCAGACGGNCAAATTGGATA (SEQ ID NO: 403) |
| 398 | HGL6.895 | | GAATGGAATCGAATGGATTGATATCAACTGGAATGGAATGGAAGGGAATGGAATGGAATGGAATT |
| | | | GAACCAAATGTNNNNGNCTTGAATGGAATG (SEQ ID NO: 404) |
| 399 | HGL6.898 | | GAATCAACATCAAACGGAAAAAAACGGAATTATCGAATGGAATCGAAGAGAATCATCGAATGGACC |
| | | | (SEQ ID NO: 405) |
| 400 | HGL6.904 | | ATGGAATCAACATCAAACGGAATCAAACGGAATTATCGAATGGAATCAAAGAGAATCATCGAACGG |
| | | | ACTCGAATGGAATCATCTAATGGAATGGAATGGAAGAATCCATGGACTCGAATGCAATCATCATCG |
| | | | AAT (SEQ ID NO: 406) |
| 401 | HGL6.905 | | GGAATGGAATGGAATGGAGCNGAATNGAANGGANNNNANTCAAATGGAATGC (SEQ ID NO: |
| | | | 407) |
| 402 | HGL6.906 | | AACATACGCAAATCAATAAATGTAATCCAGCATATAAACAGAACCAAAGACAAAAACCACATGATT |
| | | | ATCTCAATAGATGCAGAAAAGGCC (SEQ ID NO: 408) |
| 403 | HGL6.911 | | AAACGATTGGACAGGAATGGAATCACCATCGAATGGAAACGAATGGAATCTTCGAATGGAATTGAA |
| | | | TGAAATTATTGAACGGAATCAAATAGAATCATCATTGAACAGAATCAAATTGGATCAT (SEQ ID NO: |
| | | | 409) |
| 404 | HGL6.912 | | AAAAGATGCAAAAGTAGCAAATGCAATGTTAAAACAAGCAAAGAAAGAATCAGGTGGACCACATA |
| | | | GTGCAGTGCTTCTC (SEQ ID NO: 410) |
| 405 | HGL6.914 | | AACAATAAACAAACTCCAACTAGACACAATAGTCAAATTGCTGAAAATGAAATATAAAGGAACAATC |
| | | | TCGATGGTAGCCCAAGGA (SEQ ID NO: 411) |
| 406 | HGL6.915 | HGL6.916 | AGTCAATAACAAGAAGACAAACAACCCAATTACAAAATGGGATATGAATTTAATAGATGTTACTCCA |
| | | | AGGAAGATACACAAATGGCCAAC (SEQ ID NO: 412) |
| 407 | HGL6.919 | | AAAACACCTAGGAATACAGATAACAAGGGACATTAACTACCTCTTAAAGAGAACTACAAACCACTGC |
| | | | TCAAGGAAATGAGAGAGGACACAAACACATGGAAAAACATTCCATCCTCATGGATAGGAAGAATCA |
| | | | ATATTGTGAAAATGGCC (SEQ ID NO: 413) |
| 408 | HGL6.921 | | GATATATAAACAAGAAAACAACTAATCACAACTCAATATCAAAGTGCAATGATGGTGCAAAATGCAA |
| | | | GTATGGTGGGGACAGAGAAAGGATGC (SEQ ID NO: 414) |
| 409 | HGL6.923 | | ACACATATCAAACAAACAAAAGCAATTGACTATCTAGAAATGTCTGGGAAATGGCAAGATATTACA |
| | | | (SEQ ID NO: 415) |
| 410 | HGL6.924 | | GGAATCATCATATAATGGAATCGAATGGAATCAACATCAAATGGAATCAAATGGAATCATTGAACG |
| | | | GAATTGAATGGAATCGTCAT (SEQ ID NO: 416) |
| 411 | HGL6.926 | | CCCAACTTCAAATTATACTACAAGGCTACAGTAATCAAAAAAGCATAGTACTATTACAAAAACAGAC |
| | | | ACACAGGCCAATGGAATACAAT (SEQ ID NO: 417) |
| 412 | HGL6.927 | | AAACGCAGAAACAAATCAACGAAAGAACGAAGCAATGAAAGACAAAGCAACAAAAGAATGGAGTA |
| | | | AGAAAGCACACTCCACAAAGTGGAAGCAGGCTGGGACA (SEQ ID NO: 418) |
| 413 | HGL6.928 | | AACTAACACAAGAACAGAAAACCAAACATCACATGTTCTCACTCATAAGCGGGAGCTGAACAATGA |
| | | | GAACACACGGACACAGGGAGAGGAACATG (SEQ ID NO: 419) |
| 414 | HGL6.929 | | GCCACAATTTTGAAACAACCATAATAATGAGAATACACAAGACAACTCCAATAATGTGGGAAGACA |
| | | | AACTTTGCAATTCACATCATGGC (SEQ ID NO: 420) |
| 415 | HGL6.933 | | AATGGAATCAACATCAAACGGAATCAAATGGAATTATCGAATGGAATCGAAGAGAATCATCGAATT |
| | | | GTCACGAATGGAATCATCTAATGGAATGGAATGGAATAATCCATGGCCCCTATGC |
| | | | (SEQ ID NO: 421) |
| 416 | HGL6.934 | HGL6.935 | TAAACAGAACCAAAGACAAAAATCACATGATTATCTCAATAGATGCAGAAAAGGCC |
| | | | (SEQ ID NO: 422) |
| 417 | HGL6.937 | | ATCAACAGACAACAGAAACAAATCCACAAAGCACTTAGTTATTAGAACTGTCATACAGACTGTACAA |
| | | | CAACCACATTTACCAT (SEQ ID NO: 423) |
| 418 | HGL6.938 | | AATGGACTCGAATGAAATCATCATCAAACAGAATCGAATGGAATCATCTAATGGAATGGAATGGCA |
| | | | TAATCCATGGACTCGAATG (SEQ ID NO: 424) |
| 419 | HGL6.939 | | TAAAATGAAACAAATATACAACACGAAGGTTATCACCAGAAATATGCCAAAACTTAAATATGAGAAT |
| | | | AAGACAGTCTCAGGGGCCACAGAG (SEQ ID NO: 425) |
| 420 | HGL6.940 | | AAAATACAGCGTTATGAAAAGAATGAACACACACACACACACACACACACAGAAAATGT |
| | | | (SEQ ID NO: 426) |
| 421 | HGL6.942 | | TACTCTCAGAAGGGAAGCAGATATTCAGCATAAATCATATTGTTTGTACAAAGAGTCTGGGCATGGT |
| | | | GAATGACACT (SEQ ID NO: 427) |
| 422 | HGL6.943 | | CAAACAAATAGGTACCAAACAAATAACAACATAAACCTGACAACACACTTATTTACAAGAGACATCC |
| | | | CTTATATGAAAGGGTACAGAAAAGTCGATGGTAAGATGATGGGGAAAGGTATACCAACCACTAGCA |
| | | | GAAGG (SEQ ID NO: 428) |
| 423 | HGL6.944 | | TGGAATCGAATGGAATCAATATCAAACGGAAAAAAACGGAATTATCGAATGGAATCGAAAAGAATC |
| | | | ATCGAATGGGCCCGAATGGAATCATCT (SEQ ID NO: 429) |
| 424 | HGL6.945 | | ACAAATGGAATCAACAACGAATGGAATCGAATGGAAACGCCATCGAAAGGAAACGAATGGAATTA |
| | | | TCATGAAATTGAAATGGATG (SEQ ID NO: 430) |
| 425 | HGL6.947 | | GACAAGAGTTCAGAAAGGAAGACTACACAGAAATACGCATTTTAAAGTCACTGACATGGAGATGAC |
| | | | ACTTAAAACCATGAACATGGATGGG (SEQ ID NO: 431) |
| 426 | HGL6.956 | | AAAATAAACGCAAATTAAAATCACAAGATACCAACACATTCCCACGGCTAAGTACGAAGAACAAGG |
| | | | GCGAATGGTCAGAATTAAGCTCAAACCT (SEQ ID NO: 432) |
| 427 | HGL6.957 | | TAAACTGACACAAACACAGACACACAGATACACACATACATACAGAAATACACATTCACACACAGAC |
| | | | CTGGTCTTTGGAGCCAGAGATG (SEQ ID NO: 433) |
| 428 | HGL6.958 | | GATCAATAAATGTAATTCATCATATAAACAGAGAACTAAAGACAAAAACACATGATTATCGCAATAC |
| | | | ATGCAGAAAAGGCC (SEQ ID NO: 434) |
| 429 | HGL6.962 | | AGGACATGAATAGACAATTCTCAAAAGAAGATACACAAGTGGCAAACAAACACATGAAAAAAGACT |
| | | | CAACATTAGTAATGACCATGGAAATGCAAATC (SEQ ID NO: 435) |
| 430 | HGL6.963 | | ACATCAAACGGAAAAAAACGGAATTATCGAATGGAATCGAAGAGAATCATCGAATGGACC (SEQ ID |
| | | | NO: 436) |
| 431 | HGL6.965 | | AATGGACTCGAATAGAATTGACTGGAATGGAATGGACTCGAATGGAATGGAATGGAATGGAAGGG |
| | | | ACTCG (SEQ ID NO: 437) |
| 432 | HGL6.966 | | AAGAAAGACAGAGAACAAACGTAATTCAAGATGACTGATTACATATCCAAGAACATTAGATGGTCA |
| | | | AAGACTTTAAGAAGGAATACATTCAAAGGCAAAACGTCACTTACTGATTTTGGTGGAGTTTGCCACA |
| | | | TGGAC (SEQ ID NO: 438) |
| 433 | HGL6.967 | | AACATAATCCATCAAATAAACAGAACCAAAGACAAAAACCACATGATTATCTCAATAGATGCAGAAA |
| | | | AGGCCTTC (SEQ ID NO: 439) |
| 434 | HGL6.969 | | GAATGGAATCGAATGGAATGAACATCAAACGGAAAAAAACGGAATTATCGAATGGAATCAAAGAG |
| | | | AATCATCGAATGGACCCG (SEQ ID NO: 440) |
| 435 | HGL6.972 | | ATGGACTCGAATGTAATAATCATTGAACGGAATCGAATGGAATCATCATCGGATGGAAACGAATGG |
| | | | AATCATCATCGAATGGAATCGAATGGGATC (SEQ ID NO: 441) |
| 436 | HGL6.974 | | GAATGGAATCAACATCAAACGGAATCAAACGGAATTATCGAATGGAATCGAAGAGAATCATCGAAT |
| | | | GGCCACGAATGGAATCATCTAATGGAATGGAATGGAATAATCCATGG (SEQ ID NO: 442) |
| 437 | HGL6.975 | | GAAATGGAATGGAAAGGAATAAAATCAAGTGAAATTGGATGGAATGGATTGGAATGGATTGGAAT |
| | | | G (SEQ ID NO: 443) |
| 438 | HGL6.978 | | AAACGGAAAAAAAACGGAATTATCGAATGGAATCGAAGAGAATCATCGAACGAACCAGAATGGAA |
| | | | TCATCTAATGGAATGGAATGGAATAATCCATGG (SEQ ID NO: 444) |
| 439 | HGL6.981 | | ATTAACCCGAATAGAATGGAATGGAATGGAATGGAACGGAACGGAATGGAATGGAATGGAATGGA |
| | | | ATGGAATGGATCG (SEQ ID NO: 445) |
| 440 | HGL6.982 | | GCAAAACACAAACAACGCCATAAAAAACTGGGCAAAGGATATGAACAGACATTTTTCAAAACAAAA |
| | | | CATACTTATGGCCAAC (SEQ ID NO: 446) |
| 441 | HGL6.984 | | AACATCAAACGGAAAAAAACGGAATTATCGTATGGAATCGAAGAGAATCATCGAATGGACC (SEQ |
| | | | ID NO: 447) |
| 442 | HGL6.985 | | AAATCAATAAATGTAATTCAGCATATAAACAGAACCAAAGACAAAAACCACATGATTATCTCAATAG |
| | | | ATGCAGAAAAGGCCTTT (SEQ ID NO: 448) |
| 443 | HGL6.986 | | AGAATCAAATGGAATTGAATCGAATGGAATCGAATGGATTGGAAAGGAATAGAATGGAATGGAAT |
| | | | GGAATG (SEQ ID NO: 449) |
| 444 | HGL6.988 | | GAATAGAATTGAATCATCATTGAATGGAATCGAGTAGAATCATTGAAATCGAATGGAATCATCATCG |
| | | | AATGGAATTGGGTGGAATC (SEQ ID NO: 450) |
| 445 | HGL6.989 | | CACCGAATAGAATCGAATGGAACAATCATCGAATGGACTCAAATGGAATTATCCTCAAATGGAATC |
| | | | GAATGGAATTATCGAATGCAATCGAATAGAATCATCGAATAGACTCGAATGGAATCATCGAATGGA |
| | | | ATGGAATGGAACAGTC (SEQ ID NO: 451) |
| 446 | HGL6.992 | HGL6.1286 | AAATCATCATCGAATGGAATCGAATGGTATCATTGAATGGAATCGAATGGAATCATCATCAGATGG |
| | | | AAATGAATGGAATCGTCAT (SEQ ID NO: 452) |
| 447 | HGL6.997 | | GAATGGAATCGAAAGGAATAGAATGGAATGGATCGTTATGGAAAGACATCGAATGGAATGGAATT |
| | | | GACTCGAATGGAATGGACTGGAATGGAACG (SEQ ID NO: 453) |
| 448 | HGL6.998 | | GAATAGAATTGAATCATCATTGAATGGAATCGAGTAGAATCATTGAAATCGAATGGAATCATCATCG |
| | | | AATGGAATTGGGTGGAATC (SEQ ID NO: 454) |
| 449 | HGL6.1001 | | GAAAGGAATAGAATGGAATGGATCGTTATGGAAAGACATCGAATGGGATGGAATTGACTCGAATG |
| | | | GATTGGACTGGAATGGAACGGACTCGAATGGAATGGACTGGAATG (SEQ ID NO: 455) |
| 450 | HGL6.1003 | | TGGATTTCAGATATTTAACACAAAATAGTCAAAGCAGATAAATACTAGCAACTTATTTTTAATGGGTA |
| | | | ACATCATATGTTCGTGCCTT (SEQ ID NO: 456) |
| 451 | HGL6.1004 | | ACAGCAGAAAACGAACATCAGAAAATCACTCTACATGATGCTTAAATACAGAGGGCAAGCAACCCA |
| | | | AGAGAAAACACCACTTCCTAAT (SEQ ID NO: 457) |
| 452 | HGL6.1011 | | AACATACACAAATCAATAAACGTAATCCAGCTTATAAACAGAACCAAAGACAAAAACCACATGATTA |
| | | | TCTCAATAGATGCGGAAAAGGCC (SEQ ID NO: 458) |
| 453 | HGL6.1012 | | ACATCAAACGGAATCAAACGGAATTATCGAATGGAATCGAAAAGAATCATCGAACGGACTCGAATG |
| | | | GAATCATCTAATGGAATGGAATGGAAG (SEQ ID NO: 459) |
| 454 | HGL6.1013 | | ATCGAATGGAATCAACATCAAACGGAAAAAAACGGAATTATCAAATGGAATCGAAGAGAATCATCG |
| | | | AATGGACC (SEQ ID NO: 460) |
| 455 | HGL6.1014 | | GAATAATCATTGAACGGAATCGAATGGAAACATCATCGAATGGAAACGAATGGAATCATCATCGAA |
| | | | TGGAAATGAAAGGAGTCATC (SEQ ID NO: 461) |
| 456 | HGL6.1015 | | CATCAAACGGAATCAAACGGAATTATCGAATGGAATCGAAAAGAATCATCGAACGGACTCGAATGG |
| | | | AATCATCTAATGGAATGGAATGGAAGAATCCATGGACTCGAATG (SEQ ID NO: 462) |
| 457 | HGL6.1016 | | TCCAGTCGATCATCATATAGTCAGCACTTATCATACACCAAGCCGTGTGCAAGGAAAGGGAATACAA |
| | | | CCATGAACATGATAGATGGATGGTT (SEQ ID NO: 463) |
| 458 | HGL6.1017 | | ACAAACCACTGCTCAAGGAAATAAGGACACAAACAAATGGAACAACATTCCGTGCTCATGGATAGG |
| | | | AAGAATCAATATCGTGAAAATGGCCATACT (SEQ ID NO: 464) |
| 459 | HGL6.1019 | | ACAAAATTGATAGACCACTAGCAAGACTAATAAAGAAGAAAAGAGAGAAGAATCATTACCATTCAG |
| | | | GACATAGGCATGGGCAAGGAC (SEQ ID NO: 465) |
| 460 | HGL6.1024 | | AAACGGAATCAAACGGAATTATCGAATGGAATCGAAGAGAATCATCGAATGGACTCGAATGGAATC |
| | | | ATCTAATGGAATGGAATGGAAGAATCCATGG (SEQ ID NO: 466) |
| 461 | HGL6.1026 | | ATACACAAATCAATAAATGTAATCCAGCATATAAACAGAACCAAAGACAAAAACCATATGATTATCT |
| | | | CAATGGATGCAGAAAAGGCC (SEQ ID NO: 467) |
| 462 | HGL6.1027 | | AATNGAATAGAATCATCGAATGGACTCGAATGGAATCATCGANNNTANTGATGGAACAGTC (SEQ |
| | | | ID NO: 468) |
| 463 | HGL6.1030 | | TGGAATGGAATCATCGCATAGAATCGAATGGAATTACCATCGAATGGGATCGAATGGTATCAACAT |
| | | | CAAACGCAAAAAAACGGAATTATCGAATGGAATCGAAGAGAATCTTCGAACGGACCCG (SEQ ID |
| | | | NO: 469) |
| 464 | HGL6.1031 | | GAATTGAATTGAATGGAATGGAATGCAATGGAATCTAATGAAACGGAAAGGAAAGGAATGGAATG |
| | | | GAATGGAATG (SEQ ID NO: 470) |
| 465 | HGL6.1033 | | AACAGAATGGAATCAAATCGAATGAAATGGAATGGAATAGAAAGGAATGGAATGAAATGGAATGG |
| | | | AAAGGATTCGAATGGAATGCAATCG (SEQ ID NO: 471) |
| 466 | HGL6.1034 | | ATGGAATGGAATGGAATGGAATTAAATGGAATGGAAAGGAATGGAATCGAATGGAAAGGAATC |
| | | | (SEQ ID NO: 472) |
| 467 | HGL6.1037 | HGL6.1245 | GTCGAAATGAATAGAATGCAATCATCATCAAATGGAATCCAATGGAATCATCATCAAATAGAATCGA |
| | | | ATGGAATCATCAAATGGAATCGAATGGAGTCATTG (SEQ ID NO: 473) |
| 468 | HGL6.1039 | | TGGAATTATCGAAAGCAAACGAATAGAATCATCGAATGGACTCGAATGGAATCATCGAATGGAATG |
| | | | GAATGGAACAG (SEQ ID NO: 474) |
| 469 | HGL6.1045 | | AAAGGAATGGAATGCAATGGAATGCAATGGAATGCACAGGAATGGAATGGAATGGAATGGAAAG |
| | | | GAATG (SEQ ID NO: 475) |
| 470 | HGL6.1046 | | AATCTAATGGAATCAACATCNAACGGAAAAAAACGGAATTATCGAATGGAATCNAAGAGAATCATC |
| | | | NAATGGACC (SEQ ID NO: 476) |
| 471 | HGL6.1047 | | TACACAACAAAAGAAATACTCAACACAGTAAACAGACAACCTTCAGAACAGGAGAAAATATTTGCA |
| | | | AATACATCTAACAAAGGGCTAATATCCAGAATCT (SEQ ID NO: 477) |
| 472 | HGL6.1048 | | NGCAATCNTAGTNTCAGATAAAACAGACATTAAACCAACAAAGATCAAAAGAGACAAAGAAGGCC |
| | | | ANTAC (SEQ ID NO: 478) |
| 473 | HGL6.1052 | | GAATCGAATGGAATCAACATCAAACGGAAAAAAACGGAATTATCGAATGGAATCNAAAAGAATCAT |
| | | | CNAATGGACC (SEQ ID NO: 479) |
| 474 | HGL6.1055 | | ACAGTTAACAAAAACCGAACAATCTAATTACGAAATGAACAAAAGATATGAACAGACATTTCACCCG |
| | | | AGAGTATACAGGGGCCAGGCATGGT (SEQ ID NO: 480) |
| 475 | HGL6.1056 | | AATGGAATCGAATGGAATGCAATCCAATGGAATGGAATGCAATGCAATGGAATGGAATCGAACGG |
| | | | AATGCAGTGGAAGGGAATGG (SEQ ID NO: 481) |
| 476 | HGL6.1057 | | GAACACAGAAAAATTTCAAAGGAATAATCAACAGGGATTGATAACTAACTGGATTTAGAGAGCCAA |
| | | | GGCAAAGAGAATCAAAGCACAGGGCCTGAGTCGGAG (SEQ ID NO: 482) |
| 477 | HGL6.1058 | | TATACCACACAAATGCAAAAGATTATTAGCAACAATTATCAACAGCAATATGTCAACAAGTTGACAA |
| | | | ACCTAGAGGACATGGAT (SEQ ID NO: 483) |
| 478 | HGL6.1061 | | CACCATGAGTCATTAGGTAAATGCAAATCAAAACCACAATGAAATACTTCACACCCATGAAGATGGC |
| | | | TATAATAAAAAAACAGACA (SEQ ID NO: 484) |
| 479 | HGL6.1067 | | AGTTGAATAGAACCAATCCGAATGAAATGGAATGGAATGGAACGGAATGGAATTGAATGGAATGG |
| | | | AATGGAATGCAATGGA (SEQ ID NO: 485) |
| 480 | HGL6.1069 | | AAGTAATAAGACTGAATTAGTAATACAAAGTGTCTCAACAAAGAAAATTGCGGGACTGTTCATGCTC |
| | | | ATGGACAGGAAGAATCAATATCATGAAAATGGCC (SEQ ID NO: 486) |
| 481 | HGL6.1070 | | AACTCGATTGCAATGGAATGTAATGTAATGGAATGGAATGGAATTAACGCGAATAGAATGGAATGG |
| | | | AATGTAATGGAACGGAATGGAATG (SEQ ID NO: 487) |
| 482 | HGL6.1074 | | AAGCGGAATAGAATTGAATCATCATTGAATGGAATCGAGTAGAATCATTGAAATCGAATGGAATCA |
| | | | TAGAATGGAATCCAAT (SEQ ID NO: 488) |
| 483 | HGL6.1076 | | AAAGGAAAACTACAAAACACTGCTGAAAGAAATCATTGACAACACAAACAAATGGAAACACATCCC |
| | | | AAGATCATGGGTGGGTGGAATCAAT (SEQ ID NO: 489) |
| 484 | HGL6.1077 | | AATGGAATCNAAAGGAATAGAATGGAATGGATCGTTATGGAAAGATATCGAATGGAATGGAATTG |
| | | | ACTCGAATGGAATGGACTGGAATGGAACG (SEQ ID NO: 490) |
| 485 | HGL6.1078 | | TAACGGAATAATCATCGAACAGAATCAAATGGAATCATCATTGAATGGAATTGAATGGAATCTTCGA |
| | | | ATAGACATGAATGGACCATCATCG (SEQ ID NO: 491) |
| 486 | HGL6.1084 | | AAAGACCGAAACAACAACAGAAACAGAAACAAACAACAATAAGAAAAAATGTTAAGCAAAACAAA |
| | | | TGATTGCACAACTTACATGATTACTGAGTGTTCTAATGGT (SEQ ID NO: 492) |
| 487 | HGL6.1085 | | AAGATTTAAACATAAGACCTAAAACGACAAAAATCCTAGGAGAAAACCTAAGCAATACCATTCAGG |
| | | | ACATAGGCATGGGCAAAGACTTCATG (SEQ ID NO: 493) |
| 488 | HGL6.1090 | | AGAAACAGCCAGAAAACAATTATTACCTACAGCATTAAAACTATTCAAATGACAGCATATTTTTCAGC |
| | | | AGAAATCATGAAGGCCAGAAGGACGTGTCAT (SEQ ID NO: 494) |
| 489 | HGL6.1092 | | ATGTACACAAATCAATAAATGCAGTCCAGCATATAAACAGAACCAAACACAAAAACCACATGATTAT |
| | | | CTCAATAGATGCAGAAAAGGCCTTT (SEQ ID NO: 495) |
| 490 | HGL6.1093 | | AGCAACTTCAGCAAAGTCTCAGGACACAAAATCAATGTGCAAAAATCACAAGCATTCTTATACACCA |
| | | | ATAACAGACAAACAGAGAGCC (SEQ ID NO: 496) |
| 491 | HGL6.1094 | | TTGAATCGAATGGAATCGAATGGATTGGAAAGGAATAGAATGGAATGGAATGGAATTGACTCAAAT |
| | | | GGAATG (SEQ ID NO: 497) |
| 492 | HGL6.1097 | HGL6.1241 | AACGGAATCAAACGGAATTATCGAATGGAATCGAATAGAATCATCGAACGGACTCGAATGGAATCA |
| | | | TCTAATGGAATGGAATGGAAG (SEQ ID NO: 498) |
| 493 | HGL6.1098 | | AACATCACTGATCATTAGAAACACACAAATCAAAACCACAATAAGATACCATCTAACACCAGTCACA |
| | | | ATGGCTATT (SEQ ID NO: 499) |
| 494 | HGL6.1100 | | TAAGCAATTTCAGCAGTCTCAGGATACAAAATCAATGTGCAAAAATCACAAGCATTCTTATACACCA |
| | | | ACAACAGACAAACAGAGAGCCAAATCG (SEQ ID NO: 500) |
| 495 | HGL6.1101 | | AGAAAAAAACAAACAGCCCATTAAAAGGTAGACAAAGGACATGAACACTTTTCAAAAGAAGACATA |
| | | | CATGTGGCCAAACAGCATG (SEQ ID NO: 501) |
| 496 | HGL6.1103 | | ATTGGAATGGAACGGAACAGAACGGAATGGAATGGAATAGAATGGAATGGAATGGAATGGTATG |
| | | | GAATGGAATGGAATGGTACG (SEQ ID NO: 502) |
| 497 | HGL6.1104 | | AGAGCATCCACAAGGCCCAATTCAAAGAATCTGAAATAATGTATTGTTACTGCAACAGTTGTGAGTA |
| | | | CCAGTGGCATCAG (SEQ ID NO: 503) |
| 498 | HGL6.1107 | | AATCCACAAAGACAACAGAAGAAAAGACAACAGTAGACAAGGATGTCAACCACATTTTGGAAGAG |
| | | | ACAAGTAATCAAACACATGGCA (SEQ ID NO: 504) |
| 499 | HGL6.1109 | | AAACAGAACCACAGATATCTGTAAAGGATTACACTATAGTATTCAACAGAGTATGGAACAGAGTATA |
| | | | GTATTCAACAGAGTATGCAAAGAAACTAAGGCCAGAAAG (SEQ ID NO: 505) |
| 500 | HGL6.1110 | | AGCAAACAAACAAACAAACAAACAAACTATGACAGGAACAAAACGTCACATATCAACATTAACAAA |
| | | | GAATGTAAACAGCCTAAATGCTTCACTTAAAAGTTATAGACAGGGGCTGGGCATGGTGGCTCACGC |
| | | | C (SEQ ID NO: 506) |
| 501 | HGL6.1111 | | AAAAGTACAGAAGACAACAAAAAATGAGAGAGAGAAAGATAACAGACTATAGCAGCATTGGTGAT |
| | | | CAGAGCCACCAG (SEQ ID NO: 507) |
| 502 | HGL6.1114 | | TACAAGAAAATCACAGTAACATTTATAAAACACAGAAGTGTGAACACACAGCTATTGACCTTGAAAA |
| | | | CAGTGAAAGAGGGTCAGCTGTAGAACTAAGACATAAGCAAAGTTTTTCAATCAAGAATACATGGGT |
| | | | GGCC (SEQ ID NO: 508) |
| 503 | HGL6.1116 | | GAATCGAATGGAATCAACATCAAACGGAAAAAAACGGAATTATCGAATGGAATCGAAAAGAATCAT |
| | | | CGAACGGACTCGAATGGAATCATCTAATGGAATGGAATGGAAGAATCCATGG (SEQ ID NO: 509) |
| 504 | HGL6.1117 | | AATGGAATCGAATGGAATCATCATCAAATGGAATCTAATGGAATCATTGAACGGAATTGGATGGAA |
| | | | TCGTCAT (SEQ ID NO: 510) |
| 505 | HGL6.1118 | | AACGGAATCAAACGGAATTATCGAATGGAATCGAAGAGAATCATCGAATGGCCACGAATGGAATCA |
| | | | TCTAATGGAATGGAATGGAATAATCCATGGACCCGAATG (SEQ ID NO: 511) |
| 506 | HGL6.1121 | | CAACATCAAACGGAAAAAAACGGAATTATCGAATGGAATCGAAGAGAATCATCGAATGGACC (SEQ |
| | | | ID NO: 512) |
| 507 | HGL6.1122 | | CACAACCAAAGCAATGAAAGAAAAGCACAGACTTATTGAAATGAAAGTACACACCACAGAATGGGA |
| | | | GCAGGCTCAAGCAAGC (SEQ ID NO: 513) |
| 508 | HGL6.1123 | HGL6.1229 | ATCAAAGGGAATCAAGCGGAATTATCGAATGGAATCGAAGAGAATCATCGAATGGACTCGAATGG |
| | | | AATCATGTGATGGAATGGAATGGAATAATCCACGGACT (SEQ ID NO: 514) |
| 509 | HGL6.1125 | | AAGAAACAATCAAAAGGAAGTGCTAGAAATAAAACACACTGTAATAGAAAAGAAGAATGCCTTATG |
| | | | GGCTTATCAATAGACTAGACATGGCCAGG (SEQ ID NO: 515) |
| 510 | HGL6.1127 | | AGATAAGAATAAGGCAAACATAGTAATAGGGAGTTCATGAATAACACACGGAAAGAGAACTTACA |
| | | | GGGCTGTGATCAGGAAACG (SEQ ID NO: 516) |
| 511 | HGL6.1128 | | GGAATCGAATGGAATCAATATCAAACGGAGAAAAACGGAATTATCGAATGGAATCGAAGAGAATC |
| | | | ATCGAATGGACC (SEQ ID NO: 517) |
| 512 | HGL6.1130 | | TCAGACCATAGCAGATAACATGCACATTAGCAATACGATTGCCATGACAGAGTGGTTGGTG (SEQ ID |
| | | | NO: 518) |
| 513 | HGL6.1132 | | AGGAATGGACACGAACGGAATGCAATCGAATGGAATGGAATCTAATAGAAAGGAATTGAATGAAA |
| | | | TGGACTGG (SEQ ID NO: 519) |
| 514 | HGL6.1133 | | GGAAGGGAATCAAATGCAACAGAATGTAATGGAATGGAATGCAATGGAATGCAATGGAATGGAAT |
| | | | GGAATGCAATGGAATGG (SEQ ID NO: 520) |
| 515 | HGL6.1138 | | AAATTGGATTGAATCGAATCGAATGGAAAAAATGAAATCAAATGAAATTGAATGGAATCGAAATGA |
| | | | ATGTAAACAATGGAATCCAATGGAATCCAATGGAATCGAATCAAATGGTTTTGAGTGGCGTAAAAT |
| | | | G (SEQ ID NO: 521) |
| 516 | HGL6.1139 | | AAGGATTCGAATGGAATGCAATCGAATGGAATGGAATCGAACGGAATGGAATAAAATGGAAGAAA |
| | | | ACTGGCAAGAAATGGAATCG (SEQ ID NO: 522) |
| 517 | HGL6.1141 | | GAAAAATCATTGAACGGAATCGAATGGAATCATCATCGGATGGAAACGAATGGAATCATCATCGAA |
| | | | TGGAAATGAAAGGAGTCATC (SEQ ID NO: 523) |
| 518 | HGL6.1147 | | GGTTCAACTTACAATATTTTGACTTGACAACAGTGCAAAAGCAATACACGATTAGTAGAAACACACT |
| | | | TCCAATGCCCATAGGACCATTCTGC (SEQ ID NO: 524) |
| 519 | HGL6.1150 | | GGAATCGAATGGAATCAACATCAAACGGAGAAAAACGGAATTATCGAATGGAATCGAAGAGAATC |
| | | | ATCGAATGGACC (SEQ ID NO: 525) |
| 520 | HGL6.1152 | | TAACCTGATTTGCCATAATCCACGATACGCTTACAACAGTGATATACAAGTTACATGAGAAACACAA |
| | | | ACATTTTGCAAGGAAACTGTGGCCAGATG (SEQ ID NO: 526) |
| 521 | HGL6.1153 | | TAACTACTCACAGAACTCAACAAAACACTATACATGCATTTACCAGTTTATTATAAAGATACAAGTCA |
| | | | GGAACAGCCAAATGGAAGAAATGTAAATGGCAAG (SEQ ID NO: 527) |
| 522 | HGL6.1155 | | GCTCAAAGAAATCAGAAATGACACAAGCAAATGGAAAAACATGCCATGTTCATGAATATGAAGAAT |
| | | | CAATATTGTTAAAATGGCCATACTGCTCA (SEQ ID NO: 528) |
| 523 | HGL6.1157 | | AAAGAAATGTCACTGCGTATACACACACACGCACATACACACACCATGGAATACTACTCAGCTATAC |
| | | | AAAGGAATGAAATAATCCACAGCCAC (SEQ ID NO: 529) |
| 524 | HGL6.1159 | | GAATAGAACAGAATGGAATCAAATCGAATGAAATGGAATGGAATAGAAAGGAATGGAATGAAATG |
| | | | GAATGGAAAGGATTCGAATGGAATG (SEQ ID NO: 530) |
| 525 | HGL6.1162 | | TGAACGGAATCGAATGGAATCATCATCGGATGGAAACGAATGGAATCATCATCGAATGGAAATGAA |
| | | | AGGAGTCATC (SEQ ID NO: 531) |
| 526 | HGL6.1165 | | GAATAGAACGAAATGGAATGGAATGGAATGGAATGGAAAGGAATGGAATGGAATGGAACG (SEQ |
| | | | ID NO: 532) |
| 527 | HGL6.1166 | | AACGTGACATACATACAAAAAGTTTTTAGAGCAAGTGAAATTTTAGCTGCTATATGTTAATTGGTGG |
| | | | TAATCCC (SEQ ID NO: 533) |
| 528 | HGL6.1169 | | GGAATAACAACAACAACAACCAAAAGACATATAGAAAACAAACAGCACGATGGCAGATGTAAAGC |
| | | | CTACC (SEQ ID NO: 534) |
| 529 | HGL6.1174 | | GACAAAAAGAATCATCATCGAATAGAATCAAATGGAATCTTTGAATGGACTCAAAAGGAATATCGTC |
| | | | AAATGGAATCAAAAGCCATCATCGAATGGACTGAAATGGAATTATCAAATGGACTCG (SEQ ID |
| | | | NO: 535) |
| 530 | HGL6.1175 | | GTAACAAAACAGACTCATAGACCAATAGAACAGAATAGAGAATTCAGAAATAAGACTGCACTTCTAT |
| | | | GACCATGTGATCTTAGACAAACCT (SEQ ID NO: 536) |
| 531 | HGL6.1176 | | AGATAAAAAGAACAGCAGCCAAAATGACAAAAGCAAAAAGCAAAATCGTGTTAGAGCCAGGTGTG |
| | | | GTGATGTGTGCT (SEQ ID NO: 537) |
| 532 | HGL6.1178 | | GCAATCTCAGGATACAAAATCAATGTGCAAAAATCACAAGCATTCTCATACACCAATAACAGACAAA |
| | | | CAGAGCCAAATCATG (SEQ ID NO: 538) |
| 533 | HGL6.1179 | | AACCAAACCAAGCAAACAAACAAACAGTAAAAACTCAATAACAACCAACAAACAGGAAATACCAGG |
| | | | TAATTCAGATTATCTAGTTATGTGCCATAGT (SEQ ID NO: 539) |
| 534 | HGL6.1181 | | GAATGAATTGAATGCAAACATCGAATGGTCTCGAATGGAATCATCTTCAAATGGAATGGAATGGAA |
| | | | TCATCGCATAGAATCGAATGGAATTATCAACGAATGGAATCGAATGGAATCATCATCAGATGGAAA |
| | | | TGAATGGAATCGTCAT (SEQ ID NO: 540) |
| 535 | HGL6.1183 | | TGGAATGGAATCAAATCGCATGGAATCGAATGGAATAGAAAAGAATCAAACAGAGTGGAATGGAA |
| | | | TGGAATGGAATGGAATCATGCCGAATGGAATG (SEQ ID NO: 541) |
| 536 | HGL6.1184 | | GAATCCATGTTCATAGCACAACAACCAAACAGAAGAAATCACTGTGAAATAAGAAACAAAGCAAAA |
| | | | CACAGATGTCGACACATGGCA (SEQ ID NO: 542) |
| 537 | HGL6.1185 | | AAATGGAATAATGAAATGGAATCGAACGGAATCATCATCAAAAGGAACCGAATGAAGTCATTGAAT |
| | | | GGAATCAAAGGCAATCATGGTCGAATGGAATCAAATGGAAACAGCATTGAATAGAATTGAATGGA |
| | | | GTCATCACATGGAATCG (SEQ ID NO: 543) |
| 538 | HGL6.1186 | | GAATTAACCCGAATAGAATGGAATGGAATGGAATGGAACAGAACGGAACGGAATGGAATGGAATG |
| | | | GAATGGAATGGAATG (SEQ ID NO: 544) |
| 539 | HGL6.1188 | | AAGATATACAAGCAGCCAACAAACATACGAAAGAATGCTCAACATCACTAATCCTCAGAGAAATTTA |
| | | | AATCAAAACCACAATGAGTTACAATCTCATACCAGTCAGAAT (SEQ ID NO: 545) |
| 540 | HGL6.1190 | | AGAATTACAAACCACTGCTCAACAAAATAAAAGAGTACACAAACAAATGGAAGAATATTCCATGCTT |
| | | | ATGGATAGGAAGAATCAATATTGTGAAAATGGCCATACT (SEQ ID NO: 546) |
| 541 | HGL6.1192 | | CATCGAATGGACTCGAATGGAATAATCATTGAACGGAATCGAAGGGAATCATCATCGGATGGAAAC |
| | | | GAATGGAATCATCATCGAATGGAAATG (SEQ ID NO: 547) |
| 542 | HGL6.1194 | | CACCCATCTGTAGGACCAGGAAGCCTGATGTGGGAGAGAACAGCAGGCTAAATCCAGGGTTGGTCT |
| | | | CTACAGCAGAGGGAATCACAAGCCTGTTAGCAAGTGAAGAACCAACACTGGCAAGAGTGTGAAGG |
| | | | CC (SEQ ID NO: 548) |
| 543 | HGL6.1195 | | TAATGCAAACTAAAACGACAATGAGATATCAATACATAACTACCAGAAAGGCTAACAAAAAAACAG |
| | | | TCATAACACACCAAAGGCTGATGAGTGAGGATGTGCAG (SEQ ID NO: 549) |
| 544 | HGL6.1196 | | AAAGGAATCAAACGGAATTATCGAATGGAATCGAAAAGAATCATCGAACGGACTCGAATGGAATCA |
| | | | TCTAATGGAATGGAATGGAAGAATCCATGGACTCGAATG (SEQ ID NO: 550) |
| 545 | HGL6.1198 | | AGCAACTTCAGCAAAGTCTCAGGATACAAAATCAATGAGCAAAAATCACAAGCATTCTTACACACCA |
| | | | ATAACAGACAAACAGAGAGCC (SEQ ID NO: 551) |
| 546 | HGL6.1199 | | GGATATAAACAAGAAAACAACTAATCACAACTCAATATCAAAGTGCAATGATGGTGCAAAATGCAA |
| | | | GTATGGTGGGGACAGAGAAAGGATGC (SEQ ID NO: 552) |
| 547 | HGL6.1200 | | AATCAGTAAACGTAATACAGCATATAAACAGAACCAAAGACAAAAACCACATGATTATCTCAATAGA |
| | | | TGCAGAAAAGGCC (SEQ ID NO: 553) |
| 548 | HGL6.1202 | | AACATCAAACGGAAAAAAACGGAAATATCGAATGGAATCGAAGAGAATCATCGAATGGACC (SEQ |
| | | | ID NO: 554) |
| 549 | HGL6.1203 | | TAAAATGGAATCGAATGGAATCAACATCAAATGGAATCAAATGGAATCATTGAACGGAATTGAATG |
| | | | GAATCGTCAT (SEQ ID NO: 555) |
| 550 | HGL6.1204 | | AATCATCATCGAATGGAATCGAATGGTATCATTGAATGGAATCGAATGGAATCATCATCAGATGGA |
| | | | AATGAATGGAATCGTCAT (SEQ ID NO: 556) |
| 551 | HGL6.1205 | | CAATGCGTCAAGCTCAGACGTGCCTCACTACGGCAATGCGTCAAGCTCAGGCGTGCCTCACTAT |
| | | | (SEQ ID NO: 557) |
| 552 | HGL6.1206 | | AAGACAGAACACTGAAACTCAACAGAGAAGTAACAAGAACACCTAAGACAAGGAAGGAGAGGGA |
| | | | AGGCAGGCAG (SEQ ID NO: 558) |
| 553 | HGL6.1209 | | TAAGCTGATAAGCAACTTTAGCAAAGTCTCAGGATACAAAATCAATGTACAAAAATCACAAGCATTC |
| | | | TTATACACCAACAACAGACAGACGGAGAGCCAAA (SEQ ID NO: 559) |
| 554 | HGL6.1212 | | ATGAACACGAATGTAATGCAATCCAATAGAATGGAATCGAATGGCATGGAATATAAAGAAATGGAA |
| | | | TCGAAGAGAATGGAAACAAATGGAATGGAATTGAATGGAATGGAATTG (SEQ ID NO: 560) |
| 555 | HGL6.1216 | | AACAATCACTAGTCCTTAAGTAAGAGACAACACCTTTTGTCACACACAGTTTGTCCTAACTTTATCTTG |
| | | | GTAATTGGGGAGACC (SEQ ID NO: 561) |
| 556 | HGL6.1217 | | TAATGAGAAGACACAGACAACACAAAGAATCACAGAAACATGACACAGGTGACAAGAACAGGCAA |
| | | | GGACCTGCAGTGCACAGGAGCC (SEQ ID NO: 562) |
| 557 | HGL6.1218 | | TGTTGAGAGAAATTAAACAAAGCACAGATAAATGGAAAAACGTGTTCATAGATTGAAAGACTTCAT |
| | | | GTTGTATGGTGTC (SEQ ID NO: 563) |
| 558 | HGL6.1219 | | ATCAAACGGAATCAAACGGAATTATCGAATGGAATCGAAGAGAATCATCGAACGGACTCGAATGGA |
| | | | ATCATCTAATGGAATGGGATGG (SEQ ID NO: 564) |
| 559 | HGL6.1222 | | ACACAACAACCAAGAAACAACCCCATTAAGAAGTGGGAAAAATACATGAATAAACACATCTCAAAA |
| | | | GAAGACAAACAAGTGGCTAAC (SEQ ID NO: 565) |
| 560 | HGL6.1225 | | AATGGAAAGGAATCAAATGGAATATAATGGAATGCAATGGACTCGAATGGAATGGAATGGAATGG |
| | | | ACCCAAATGGAATGGAATGGAATGGAATG (SEQ ID NO: 566) |
| 561 | HGL6.1226 | | GGAATACAACGGAATGGAATCGAAAAAAATGGAAAGGAATGAAATGAATGGAATGGAATGGAAT |
| | | | GGAATGGATGGGAATGGAATGGAATGG (SEQ ID NO: 567) |
| 562 | HGL6.1227 | | GAATCAAGCGGAATTATCGAATGGAATCGAAGAGAATCATCGAAAGGACTCGAATGGAATCATCTA |
| | | | ATGGAATGGAATGGAATAATACACGGACC (SEQ ID NO: 568) |
| 563 | HGL6.1232 | | AACAACAACAACAACAGGAAAACAACCTCAGTATGAAGACAAGTACATTGATTTATTCAACATTTAC |
| | | | TGATCACTTTTCAGGTGGTAGGCAGACC (SEQ ID NO: 569) |
| 564 | HGL6.1233 | | AAGATAACCTGTGCCCAGGAGAAAAACAATCAATGGCAACAAAAGCAGAAACAACACAAATGATAC |
| | | | AATTAGCAGACAGAAACATTGAGATTGCTATT (SEQ ID NO: 570) |
| 565 | HGL6.1234 | | AATGGACTCCAATGGAATAATCATTGAACGGAATCNAATGGAATCATCATCGGATGGAAATGANTG |
| | | | GAATCNTCNTCNAATGGAATCN (SEQ ID NO: 571) |
| 566 | HGL6.1237 | | ANNCNNTAAACGTAATCCATCACATAAACANGANCNAANAGNNNAACCGCNNGATTATCTCNNN |
| | | | NNNTGCNNAAAAGGCC (SEQ ID NO: 572) |
| 567 | HGL6.1240 | HGL6.1277 | TAATTGATTCGAAATTAATGGAATTGAATGGAATGCAATCAAATGGAATGGAATGTAATGCAATGG |
| | | | AATGTAATAGAATGGAAAGCAATGGAATG (SEQ ID NO: 573) |
| 568 | HGL6.1242 | | AAAGGAATGGACTTGAACAAAATGAAATCGAACGATAGGAATCGTACAGAACGGAAAGAAATGGA |
| | | | ACGGAATGGAATG (SEQ ID NO: 574) |
| 569 | HGL6.1243 | | AGCAACTTCAGCAAAATCTCAGGATACAAAATCAATGTACAAAAATCACAAGCATTCTTATACACCA |
| | | | ACAACAGACAAACAGAGAGCC (SEQ ID NO: 575) |
| 570 | HGL6.1247 | | TGAGCAGGGAACAATGCGGATAAATTTCACAAATACAATGTTGAGCAAAAGAAAGACACAAAANA |
| | | | ATACACACATACACACCATATGGGCTAGG (SEQ ID NO: 576) |
| 571 | HGL6.1254 | | AATGGAATGGAATGTACAAGAAAGGAATGGAATGAAACCGAATGGAATGGAATGGACGCAAAATG |
| | | | AATGGAATGGAAGTCAATGG (SEQ ID NO: 577) |
| 572 | HGL6.1260 | | AAGTTCAAACATCAGTATTAACCTTGAACATCAATGGCCTACATGCATCACTTAAAACATACAGACA |
| | | | GGCAAATTGGGTTAAGAAAACAAACAAGCAAACAAAACATGTTCCAAACATTTGTTGGCTAT |
| | | | (SEQ ID NO: 578) |
| 573 | HGL6.1262 | | GGAATAATCATTGAACGGAATCGAATGGAATCATCATCGGATGGAAACGAATGGAATCATCATCGA |
| | | | ATGGAAATGAAAGGAGTCATC (SEQ ID NO: 579) |
| 574 | HGL6.1264 | | GGAACGAAATCGAATGGAACGGAATAGAATAGACTCGAATGTAATGGATTGCTATGTAATTGATTC |
| | | | GAATGGAATGGAATCG (SEQ ID NO: 580) |
| 575 | HGL6.1265 | | TGAAAGGAATAGACTGGAACAAAATGAAATCGAATGGTAGGAATCATACAGAACAGAAAGAAATG |
| | | | GAACGGAATGGAATG (SEQ ID NO: 581) |
| 576 | HGL6.1266 | | AACCCGAATAGAATGGAATGGAATGGAATGGAACGGAACGGAATGGAATGGAATGGATTGGAAT |
| | | | GGAATGGAATG (SEQ ID NO: 582) |
| 577 | HGL6.1267 | | AAAGAGAATCAAATGGAATTGAATCGAATGGAATCGAATGGATTGGAAAGGAATAGAATGGAATG |
| | | | GAATGGAATGGAATGGAATGGAATG (SEQ ID NO: 583) |
| 578 | HGL6.1269 | | AAAACACACAAACATACATGTGGATGCACATATAAACATGCACATACACACACACATAAATGCACAA |
| | | | ACACACTTAACACAAGCACACATGCAAACAAACACATGG (SEQ ID NO: 584) |
| 579 | HGL6.1270 | | AATGGAATCATCAGTAATGGAATGGAAAGGAATGGAAAGGACTGGAATGGAATGGAATGGAATG |
| | | | GAATGG (SEQ ID NO: 585) |
| 580 | HGL6.1271 | | GGAACAAAATGAAATCGAACGGTAGGAATCGTACAGAACGGAAAGAAATGGAACGGAATGGAAT |
| | | | GCACTCAAATGGAAAGGAGTCCAATGGAATCGAAAGGAATAGAATGGAATGG (SEQ ID NO: 586) |
| 581 | HGL6.1272 | | AGAATGAGATCAAGCAGTATAATAAAGGAAGAAGTAGCAAAATTACAACAGAGCAGTGAAATGGA |
| | | | TATGCTTTCTGGCAATAATTGTGAAAGGTCTGGTAATGAGAAAGTAGCAACAGCTAGTGGCTGCCAC |
| | | | (SEQ ID NO: 587) |
| 582 | HGL6.1273 | | AACAAATGGAATCAACATCGAATGGAATCGAATGGAAACACCATCGAATTGAAACGAATGGAATTA |
| | | | TCATGAAATTGAAATGGATGGACTCATCATCG (SEQ ID NO: 588) |
| 583 | HGL6.1278 | | TAACATGCAGCATGCACACACGAATACACAACACACAAACATGTATGCACGCACACGTGAATACACA |
| | | | ACACACACAAACATGCATGCATGCATACATGAATACACAGCACACAAATATCCAGCAT (SEQ ID |
| | | | NO: 589) |
| 584 | HGL6.1279 | | GAATGGAATCAACATCAAACGGAAAAAAAACGGAATTATCGAATGGAATCGAATAGAATCATCGAA |
| | | | TGGACC (SEQ ID NO: 590) |
| 585 | HGL6.1281 | | AATCGAATGAAATGGAGTCAAAAGGAATGGAATCGAATGGCAAGAAATCGAATGTAATGGAATCG |
| | | | CAAGGAATTGATGTGAACGGAACGGAATGGAAT (SEQ ID NO: 591) |
| 586 | HGL6.1282 | | AATGGAATTGAACGGAAACATCAGCGAATGGAATCGAAAGGAATCATCATGGAATAGATTCGAATG |
| | | | GAATGGAAAGGAATGGAATGGAATG (SEQ ID NO: 592) |
| 587 | HGL6.1283 | | ATGGAATCAACATCAAACAGAATCAAACGGAATTATCGAATGGAATCGAAGACAATCATCGAATGG |
| | | | ACTCGAATGGAATCATCTAATGGAATGGAATGGAAGAATCCATGGTCTCGAATGCAATCATCATCG |
| | | | (SEQ ID NO: 593) |
| 588 | HGL6.1284 | | GAATAATCATTGAACGGAATCGAATGGAATCATCTTCGGATGGAAACGAATGGAATCATCATCGAA |
| | | | TGGAAATGAAAGGAGTCATC (SEQ ID NO: 594) |
| 589 | HGL6.1288 | | AATGGACTCGAATGGAATAATCATTGAACGGAATCGAATGGAATCATCATCGGATGGAAATGAGTG |
| | | | GAATCATCATCGAATGGAATCG (SEQ ID NO: 595) |
| 590 | HGL6.1290 | | AAATGAAATCGAACGGTAGGAATCGTACAGAACGGAAAGAAATGGAACGGAATGGAATGCAATCG |
| | | | AATGGAAAGGAGTCCAATGGAAGGGAATCGAAT (SEQ ID NO: 596) |
| 591 | HGL6.1291 | | TACCAAACATTTAAAGAACAAATATCAATCCTACGCAAACCATTCTGAAACACAGAGATGGAGGATA |
| | | | TACAGCGAAACTCATTCTACATGGCC (SEQ ID NO: 597) |
| 592 | HGL6.1292 | | TATTGGAATGGAATGGAATGGAGTCGAATGGAACGGAATGCACTCGAATGGAAGGCAATGCAATG |
| | | | GAATGCACTCAACAGGAATAGAATGGAATGGAATGGAATGG (SEQ ID NO: 598) |
| 593 | HGL6.1294 | | AGAGAGTATTCATCATGAGGAGTATTACTGGACAAATAATTCACAAACGAACAAACCAAAGCGATC |
| | | | ATCTTTGTACTGGCTGGCTA (SEQ ID NO: 599) |
| 594 | HGL6.1295 | | GGAATTTAATAGAATGTACCCGAATGGAACGGAATGGAATGGAATTGTATGGCATGGAATGGAA |
| | | | (SEQ ID NO: 600) |
| 595 | HGL6.1298 | | GCAATCCANTANAATGGAATCGAATGGCATGGAATATAAAGAAATGGAATCGAAGAGAATGGAGA |
| | | | CAAATGGAATGGAATTGAATGGAATGGAATTG (SEQ ID NO: 601) |
| 596 | HGL6.1299 | | AATGGAATCGAATGGAATCATCATCAAATGGAATCTAATGGAATCATTGAACGGAATTAAATGGAA |
| | | | TCGTCATCGAATGAATTCAATGCAATCAACGAATGGTCTCGAATGGAACCAC (SEQ ID NO: 602) |
| 597 | HGL6.1300 | | AATTGCAAAAGAAACACACATATACACATATAAAACTCAAGAAAGACAAAACTAACCTATGGTGATA |
| | | | GAAATCAGAAAAGTACAGTACATTGGTTGTCTTGGTGGG (SEQ ID NO: 603) |
| 598 | HGL6.1303 | | TGACATCATTATTATCAAGAAACATTCTTACCACTGTTACCAACTTCCCAACACAGACTATGGAGAGA |
| | | | GAGATAAGACAGAATAGCATT (SEQ ID NO: 604) |
| 599 | HGL6.1305 | | GGAATCTATAATACAGCTGTTTATAGCCAAGCACTAAATCATATGATACAGAAAACAAATGCAGATG |
| | | | GTTTGAAGGGTGGG (SEQ ID NO: 605) |
| 600 | HGL6.1308 | | AAAGAATTGAATTGAATAGAATCACCAATGAATTGAATCGAATGGAATCGTCATCGAATGGAATCG |
| | | | AAGGGAATCATTGGATGGGCTCA (SEQ ID NO: 606) |
| 601 | HGL6.1311 | | ATCATCGAATGGAATCGAATGGAATCAATATCAAACGGAAAAAAACGGAATTATCGAATGGAATCG |
| | | | AATAGAATCATCGAATGGACC (SEQ ID NO: 607) |
| 602 | HGL6.1314 | | GAATGAAATCGTATAGAATCATCGAATGCAACTGAATGGAATCATTAAATGGACTTGAAAGGAATT |
| | | | ATTATGGAATGGAATTG (SEQ ID NO: 608) |
| 603 | HGL6.1316 | | TAAGCAACTTCAGCAAAGTCTCAGGATACAAAATCAATGTGCAAAAATCTCAAGCATTCTTATACAC |
| | | | GAACAACAGACAAACAGAGAGCT (SEQ ID NO: 609) |
| 604 | HGL6.1317 | | ACTCAAAAGGAATTGATTCGAATGGAATAGAATGGCAAGGAATAGTATTGAATTGAATGGAATGGA |
| | | | ATGGACCCAAATG (SEQ ID NO: 610) |
| 605 | HGL6.1319 | | GAATGGAATTTAAAGGAATAGAATGGAAGGAATCGGATGGAATGGAATGGAATAGAATGGAGTCG |
| | | | AATGGAATAGAATCGAATGGAATGGCATTG (SEQ ID NO: 611) |
| 606 | HGL6.1323 | | AACAAAAAATGAGTCAAGCCTTAAATAAAATCAGAGCCAAAAAAGAAGACATTACATCTGATAAGA |
| | | | CAAAAATTCAAAGGACCATC (SEQ ID NO: 612) |
| 607 | HGL6.1324 | | AACCCAGTGGAATTGAATTGAATGGAATTGAATGGAATGGAAAGAATCAATCCGAGTCGAATGGAA |
| | | | TGGTATGGAATGGAATGGCATGGAATCAAC (SEQ ID NO: 613) |
| 608 | HGL6.1327 | | ATCAACATCAAACGGAAAAAAAACGGAATTATCGAATGGAATCGAAGAGAATCATCGAATGGACC |
| | | | (SEQ ID NO: 614) |
| 609 | HGL6.1331 | | AAGGAATGGAATGGTACGGAATAGAATGGAATGGAACGAATTGTAATGGAATGGAATTTAATGGA |
| | | | ACGGAATGGAATGGAATGGAATCAACG (SEQ ID NO: 615) |
| 610 | HGL6.1334 | | AACGGAATGGAAAGCAATTTAATCAAATGCAATACAGTGGAATTGAAGGGAATGGAATGGAATGG |
| | | | C (SEQ ID NO: 616) |
| 611 | HGL6.1335 | | AATCGAATGGAACGGAATAGAATAGACTCGAATGTAATGGATTGCTATGTAATTGATTCGAATGGA |
| | | | ATGGAATCGAATGGAATGCAATCCAATGGAATGGAATGCAATGCAATGGAATGGAATCGAACGGA |
| | | | ATGCAGTGGAAGGGAATGG (SEQ ID NO: 617) |
| 612 | HGL6.1336 | | TAGCAACATTTTAGTAACATGATAGAAACAAAACAGCAACATAGCAATGCAATAGTAACACAACAGC |
| | | | AACATCATAACATGGCAGCA (SEQ ID NO: 618) |
| 613 | HGL6.1337 | | GGACAAATTGCTAGAAATAAACAAATTACCAAAAATGATTCAAGTAGAGACAGAGAATCAAAATAG |
| | | | AACTACACATAAGTGGGCCAAG (SEQ ID NO: 619) |
| 614 | HGL6.1340 | | AAAATAGAATGAAAGAGAATCAAATGGAATTGAATCGAATGGAATCGAATGGATTGGAAAGGAAT |
| | | | AGAATGGAATGGAATGGAATG (SEQ ID NO: 620) |
| 615 | HGL6.1342 | | AGCAAACAAGTGAATAAACAAGCAAACAAGTGAACAAGCAAACAAGTGAATAAACAAGCAAACAA |
| | | | GTGAACAAGCAAACAAGTGAATAAACAAGCAAACAAGTGAACAAGGAAACAAGTGAATAAACAAA |
| | | | GGCTCT (SEQ ID NO: 621) |
| 616 | HGL6.1346 | | AATGGAATCAACACGAGTGCAATTGAATGGAATCGAATGGAATGGAATGGAATGGAATGAATTCA |
| | | | ACCCGAATGGAATGGAAAGGAATGGAATC (SEQ ID NO: 622) |
| 617 | HGL6.1347 | | AATATACGCAAATCAATAAATGTAATCCAGCATATAAACAGTACTAAAGACAAAAACCACATGATTA |
| | | | TCTCAATAGATGCAGAAAAGGCC (SEQ ID NO: 623) |
| 618 | HGL6.1352 | | GAATCGAATGGAATCAACATCAAACGGAAAAAAACGGAATTATCGAATGGAATCGAAGAGNNNNN |
| | | | NCGAATGGACC (SEQ ID NO: 624) |
| 619 | HGL6.1354 | | AACACGAATGTAATGCAATCCAATAGAATGGAATCGAATGGCATGGAATATAAAGAAATGGAATCG |
| | | | AAGAGAATGGAAACAAACGGAATGGAATTGAATGGAATGGAATTGAATGGAATGGGAACGAATG |
| | | | GAGTGAAATTG (SEQ ID NO: 625) |
| 620 | HGL6.1355 | | GAATGGAACGGAATAGAACAGACTCGAATGTAATGGATTGCTATGTAATTGATTCGAATGGAATGG |
| | | | AATCGAATGGAATGCAATCCAATGGAATGGAATGCAATGCAATGGAATGGAATCGAATGGAATGC |
| | | | AGTGGAAGGGAATGG (SEQ ID NO: 626) |
| 621 | HGL6.1356 | | GAATCGAATGGAATCAATATCAAACGGAAAAAAACGGAATTATCGAATGGAATCGAAGAGAATCAT |
| | | | CGAATGGACC (SEQ ID NO: 627) |
| 622 | HGL6.1359 | | TAAACAACGAGAACACATGAACACAAAGAGGGGAACAACAGACACCAAGACCTTCTTGAGGGTGG |
| | | | AGGATGGGAGGAGGGAG (SEQ ID NO: 628) |
| 623 | HGL6.1360 | | AGCAACTTCAGCAGTCTCAGTATACAAAAACAATGTGCAAAAATCACAAGCATTCCTATATGCCAAT |
| | | | AACAGACAAACAGAGAGCC (SEQ ID NO: 629) |
| 624 | HGL6.1361 | | ATCAAAAGAAAAGCAACCTAACAAATACGGGAAGAATATTTGAATAGACATTTCACAGGAAAAGAT |
| | | | ATATGAATGGCCAAAAAGCAAATGAAAAG (SEQ ID NO: 630) |
| 625 | HGL6.1364 | | ATAAACATCAAACGGAATCAAACGGAATTATCGAATGGAATCGAAGAGAATAATCGAATGGACTCA |
| | | | AATGGAGTCATCTAATGGAATGGTATGGAAGAATCCATGGACTCCAACGCAATCATCAGCGAATGG |
| | | | AATC (SEQ ID NO: 631) |
| 626 | HGL6.1365 | | AAAAGAAAAGACAAAAGACACCAATTGCCAATACTGAAATGAAAAAACAGGTAATAACTATTGATC |
| | | | CCATGGACATTAAAATGATGTTGAAGGAACACCAC (SEQ ID NO: 632) |
| 627 | HGL6.1368 | | AGCAATAACCAAACAACCTCATTAAAAAGTAGGCAAAGGACATAAACAGACACTTTTCAAAAGAAG |
| | | | ACATACACGTGGCCAACAAACATATG (SEQ ID NO: 633) |
| 628 | HGL6.1370 | | AGCAACTTCAGCAAAGTCTCAGGATACAAAATCGATGTGCAAAAATCACAAGCATTCTTATACACCA |
| | | | ATAACAGGCAAACAGAGAGCC (SEQ ID NO: 634) |
| 629 | HGL6.1371 | | GTCATATTTGGGATTTATCATCTGTTTCTATTGTTGTTGTTTTAGTACACACAAAGCCACAATAAATAT |
| | | | TCTAGGCT (SEQ ID NO: 635) |
| 630 | HGL6.1373 | | ATCATCGAATGGAATAGAATGGTATCAACATCAAACGGAGAAAAACGGAATTATCGAATGGAATCG |
| | | | AAGAGAATCTTCGAACGGACC (SEQ ID NO: 636) |
| 631 | HGL6.1374 | | AAATAAGCCAACGGTCATAAATTGCAAAGCCTTTTACAATCCAAACATGATGGAAACGATATGCCAT |
| | | | TTTGAAGGTGATTTGAAAAGCACATGGTTT (SEQ ID NO: 637) |
| 632 | HGL6.1375 | | GAATGGAATCATCGCATAGAATCGGATGGAATTATCATCGAATGGAATCGAATGGTATCAACATCA |
| | | | AACGGAAAAAAACGGAATTATCGAATGGAATCGAATTGAATCATCGAACGGACCCG (SEQ ID |
| | | | NO: 638) |
| 633 | HGL6.1378 | | AATGGACTCGAATGGAATAATCATTGAACGGAATCGAATGGAATCATCATCGGATGGAAATGAATG |
| | | | GAATAATCCATGGACTCGAATGCAATCATCATCGAATGGAATCGAATGGAATCATCGAATGGACTC |
| | | | G (SEQ ID NO: 639) |
| 634 | HGL6.1379 | | AATGCAATCATCAACTGGCTTCGAATGGAATCATCAAGAATGGAATCGAATGGAATCATCGAATGG |
| | | | ACTC (SEQ ID NO: 640) |
| 635 | HGL6.1380 | | AAGAGACCAATAAGGANTANGTAAGCAACANGAGGAAGGAGANANGGGCAAGAGAGATGACCA |
| | | | GAGTT (SEQ ID NO: 641) |
| 636 | HGL6.1382 | | TGGAATCATCATAAAATGGAATCGAATGGAATCAACATCAAATGGAATCAAATGGAATCATTGAAC |
| | | | GGAATTGAATGGAATCGTCAT (SEQ ID NO: 642) |
| 637 | HGL6.1383 | | GGAATCATCGCATAGAATCGAATGGAATTATCATCGAATGGAATCGAATGGAATCAACATCAAACG |
| | | | AAAAAAAACCGGAATTATCGAATGGAATCGAAGAGAATCATCGAACGGACC (SEQ ID NO: 643) |
| 638 | HGL6.1384 | | AAATCATCATCGAATGGGATCGAATGGTATCCTTGAATGGAATCGAATGGAATCATCATCAGATGG |
| | | | AAATGAATGGAATCGTCAT (SEQ ID NO: 644) |
| 639 | HGL6.1386 | | GGAATGTAATAGAACGGAAAGCAATGGAATGGAACGCACTGGATTCGAGTGCAATGGAATCTATT |
| | | | GGAATGGAATCGAATGGAATGGTTTGGCATGGAATGGAC (SEQ ID NO: 645) |

1-6. (canceled)

7. A method for identifying polypeptide ligands for a target of interest, comprising
(a) contacting a nucleic acid library with reagents for RNA transcription under conditions to promote transcription of RNA from the double stranded nucleic acid constructs, resulting in an RNA expression product, wherein the nucleic acid library comprises a plurality of recombinant double stranded DNA constructs comprising:
(i) a first restriction enzyme recognition site
(ii) one or more translation enhancement elements downstream of the first restriction enzyme recognition site
(iii) a start codon downstream of the one or more translation enhancement elements;
(iv) a random region of at least about 18 to about 60 nucleotides immediately downstream from the start codon, wherein the peptide encoded by the random region of each linear recombinant double stranded DNA construct is capable of binding to the same target
(v) a protease cleavage site downstream of the random region; and
(vi) a second restriction enzyme recognition site downstream of the protease cleavage site
(b) contacting the RNA expression product with reagents for protein expression under conditions to promote translation of detectable polypeptide;
(c) incubating the detectable polypeptide with a target of interest under suitable conditions to promote binding of the detectable polypeptide to the target, to produce binding complexes; and
(d) analyzing the detectable polypeptides bound to the target.

8. The method of claim 7, further comprising removing unbound polypeptides prior to step (d).

9. The method of claim 8, wherein removing unbound polypeptides comprises contacting the binding complexes with a size-limiting membrane, wherein detectable polypeptides bound to the target are retained on the membrane, and unbound polypeptides pass through pores of the membrane.

10. The method of claim 9, wherein the size-limiting membrane comprises regenerated cellulose.

11.-14. (canceled)

15. A separation device, comprising:
(a) a multiwell plate;
(b) a regenerated cellulose layer below the multiwell plate, wherein the regenerated cellulose layer has a pore size suitable to retain peptides bound to a target, but not to retain unbound peptides; and
(c) a nylon membrane layer below the regenerated cellulose layer, wherein the nylon membrane layer has a pore size suitable to retain unbound peptides.

16-20. (canceled)

21. A method for identifying peptide ligands for a target of interest, comprising
(a) contacting a nucleic acid library with reagents for RNA transcription under conditions to promote transcription of RNA from the double stranded nucleic acid constructs, resulting in an RNA expression product, wherein the nucleic acid library comprises a plurality of recombinant double stranded DNA constructs comprising:
(i) a promoter;
(ii) one or more translation enhancement elements downstream of the promoter and upstream of the start codon;
(iii) a start codon downstream of the one or more translation enhancing elements;
(iv) a random region of at least about 18 to about 60 nucleotides immediately downstream from the start codon;
(v) a protease cleavage site downstream of the random region;
(vi) a unique restriction enzyme recognition site downstream of the protease cleavage site and
(vii) a heterologous cross-linking region downstream of the unique restriction enzyme recognition site;
(b) contacting the RNA expression product with reagents for ligating a linker containing a puromycin residue to the 3′ end of the RNA expression product, resulting in a labeled RNA expression product;
(c) contacting the labeled RNA expression product with reagents for protein expression under conditions to promote protein translation from the labeled RNA expression product, resulting in a RNA-polypeptide fusion product;
(d) reverse transcribing the RNA-polypeptide fusion products to produce an RNA-polypeptide fusion product-cDNA heteroduplex;
(e) incubating the RNA-polypeptide fusion product-cDNA heteroduplexes with a target of interest;
removing RNA-polypeptide fusion product-cDNA heteroduplexes that are not bound to the target of interest, resulting in binding complexes; and
(g) amplifying ligand-bound RNA-polypeptide fusion product-cDNA heteroduplexes in the binding complexes, to produce double stranded DNA constructs that can be used to identify the peptide ligands bound to the target of interest.

22. The method of claim 21, wherein the double stranded DNA constructs comprise:
(a) a first restriction enzyme recognition site;
(b) one or more translation enhancement elements downstream of the first restriction enzyme recognition site;
(c) a start codon downstream of the one or more translation enhancement elements;
(d) a random region of at least about 18 to about 60 nucleotides immediately downstream from the start codon, wherein the peptide encoded by the random region of each linear recombinant double stranded DNA construct is capable of binding to the same target;
(d) a protease cleavage site downstream of the random region; and
(e) a second restriction enzyme recognition site downstream of the protease cleavage site.

23. (canceled)

24. The method of claim 21, wherein the target is incubated with an excess of the RNA-polypeptide fusion product-cDNA heteroduplexes.

25. The method of claim 21, wherein removing RNA-polypeptide fusion product-cDNA heteroduplexes that are not bound to the target of interest comprises incubating the in the presence of a denaturant.

26. (canceled)

27. The method of claim 21, further comprising in vitro translation of peptides encoded by the cloned double stranded DNA construct, wherein the peptides are expressed as N-terminal fusions with the peptide purification tag.

28.-30. (canceled)

31. The method of claim 27, further comprising incubating the in vitro translated peptides with the target of interest to form a second binding complex, and removing unbound in vitro translated peptides.

32. The method of claim 31, wherein removing unbound in vitro translated peptides comprises passing the second binding complex through a size-limiting membrane.

33.-39. (canceled)


SEQUENCE LISTING (645 sequences)

SEQ ID NO: 1 - DNA, 63 bp, Artificial Sequence
  Synthetic
atngaatnnaanngaatggannnnaanggaatngaatnnaatggaatggaanngantngaatg

SEQ ID NO: 2 - DNA, 75 bp, Artificial Sequence
  Synthetic
aannggaatnnaatngaatnnaanngaatggannnnaanggaatngaatnnaatggaatggaanngantngaatg

SEQ ID NO: 3 - DNA, 85 bp, Artificial Sequence
  Synthetic
aaanagaatcaannggaatnnaatngaatnnaanngaatggannnnaanggaatngaatnnaatggaatggaanngantngaatg

SEQ ID NO: 4 - DNA, 13 bp, Artificial Sequence
  Synthetic
nnatcnntaaang

SEQ ID NO: 5 - DNA, 13 bp, Artificial Sequence
  Synthetic
aaatcaataaatg

SEQ ID NO: 6 - DNA, 12 bp, Artificial Sequence
  Synthetic
tagccggtgacc

SEQ ID NO: 7 - DNA, 75 bp, Artificial Sequence
  Synthetic
aatcgaatggaatcaacatcaaacggaaaaaaacggaattatcgaatggaatcgaagagaatcatcgaatggacc

SEQ ID NO: 8 - DNA, 78 bp, Artificial Sequence
  Synthetic
tggaatcgaatggaatcaacatcaaacggaaaaaaacggaattatcgaatggaatcgaagagaatcatcgaatggacc

SEQ ID NO: 9 - DNA, 103 bp, Artificial Sequence
  Synthetic
agcattcatatcttgcagtgttgggaaagagtgagaggttgtgatgtcaagaaggataggtcagaagtggaaggtatgggggattgtgcctgctgtcatggct

SEQ ID NO: 10 - DNA, 148 bp, Artificial Sequence
  Synthetic
ggaacgaaatcgaatggaacggaatagaatagactcgaatgtaatggattgctatgtaattgattcgaatggaatggaatcgaatggaatgcaatccaatggaatggaatgcaatgcaatgaatggaatggaatggaatggaatggaa

SEQ ID NO: 11 - DNA, 147 bp, Artificial Sequence
  Synthetic
ggaacgaaatcgaatggaacggaatagaatagactcgaatgtaatggattgctatgtaattgattcgaatggaatggaatcgaatggaatgcaatccaatggaatggaatgcaatgcaatgaatggaatggaatggaatggaatgga

SEQ ID NO: 12 - DNA, 86 bp, Artificial Sequence
  Synthetic
tacgcaaatcgataaatgtaatccagcatataaacagaaccaaagacaaaaaccacatgattatctcaatagatgcagaaaaggcc

SEQ ID NO: 13 - DNA, 105 bp, Artificial Sequence
  Synthetic
actcgaatgcaatcaacatcaaacggaatcaaacggaattatcgaatggaatcgaagagaatcatcgaacggactcgaatggaatcatctaatggaatggaatgg

SEQ ID NO: 14 - DNA, 81 bp, Artificial Sequence
  Synthetic
gaaattccaattaaaatgaaatcgacttatcttaacaaatatagcaatgctgacaacacttctccggatatgggtactgct

SEQ ID NO: 15 - DNA, 91 bp, Artificial Sequence
  Synthetic
aaggaaaagtaaaaggaacttaacaccttcaagaaaagacagacaaataacaaaacagcagtttgatagaatgagatatcaggggatggca

SEQ ID NO: 16 - DNA, 63 bp, Artificial Sequence
  Synthetic
atcaacatcaaacggaaaaaacggaattatcgaatggaatcgaagagaatcatcgaacggacc

SEQ ID NO: 17 - DNA, 139 bp, Artificial Sequence
  Synthetic
aaagaaagacagagaacaaacgtaattcaagatgactgattacatatccaagaacattagatggtcaaagactttaagaaggaatacattcaaaggcaaaaagtcacttactgattttggtggagtttgccacatggac

SEQ ID NO: 18 - DNA, 79 bp, Artificial Sequence
  Synthetic
aagggaattgaatagaatgaatccgaatggaatggaatggaatggaatggaatggaatggaatggaatggaatggaatg

SEQ ID NO: 19 - DNA, 84 bp, Artificial Sequence
  Synthetic
gaatggaatcgaatcaaattaaatcaaatggaatgcaatagaagggaatacaatggaatagaatggaatggaatggaatggact

SEQ ID NO: 20 - DNA, 104 bp, Artificial Sequence
  Synthetic
acagcaagagagaaataaaacgacaagaaaactacaaaatgcctatcaatagttactttaaatatcagtggaccaaatcagtgaaacaaaagacacagagtggc

SEQ ID NO: 21 - DNA, 80 bp, Artificial Sequence
  Synthetic
tagcaggaaacagcaaactcaaattaagtaatttcaagagcgtatcatcaatgaactattttcaaagatgtgggcaagat

SEQ ID NO: 22 - DNA, 78 bp, Artificial Sequence
  Synthetic
aaacggaattatcaaatggaatcgaagagaatcatcgaacggactcgaatggaatcatctaatggaatggaatggaag

SEQ ID NO: 23 - DNA, 136 bp, Artificial Sequence
  Synthetic
gaatgaaatgaaatcaaatngaatgtacatgaatggaatagaaaagaatgcatctttctcgaacggaagtgcattgaatggaaaggaatctactggaatggattcgaatggaatggaangggatggaatggtatgg

SEQ ID NO: 24 - DNA, 97 bp, Artificial Sequence
  Synthetic
aatggactcgaatgaaatcatcatcaaacggaatcgaatggaatcattgaatggaaaggatgggatcatcatggaatggaaacgaatggaatcactg

SEQ ID NO: 25 - DNA, 104 bp, Artificial Sequence
  Synthetic
aatggaatcattgaatggaatggaatggaatcatcaaagaaaggaatcgaagggaatcatcgaatggaatcaaacggaatcatcgaatggaatggaatggaatg

SEQ ID NO: 26 - DNA, 73 bp, Artificial Sequence
  Synthetic
agcagaagaaataactgaaatcagagtgaaactgaatcaaattgagatgcaaaaatacatacgaaatggccag

SEQ ID NO: 27 - DNA, 79 bp, Artificial Sequence
  Synthetic
agttaatccgaatagaatggaatggaatgcaatggaacggaatggaacggaatggaatggaatggaatggaatggaatg

SEQ ID NO: 28 - DNA, 165 bp, Artificial Sequence
  Synthetic
atggaatcaacatcaaacggaatcaaacggaattatcgaatggaatcgaagagaatcatcgaacggattcgaatggaatcatctaatggaatggaatggaagaatccatggactcgaatgcaatcatcagcgaatggaatcgaatggaatcatcgaatggactcg

SEQ ID NO: 29 - DNA, 84 bp, Artificial Sequence
  Synthetic
aaaggaatggactggaacaaaatgaaatcgaacggtaggaatcgtacagaacggacagaaatggaacggcatggaatgcactcg

SEQ ID NO: 30 - DNA, 66 bp, Artificial Sequence
  Synthetic
aaatcaacaacaaacggaaaaaaaaggaattatcgaatggaatcaaagagaatcatcgaatggacc

SEQ ID NO: 31 - DNA, 86 bp, Artificial Sequence
  Synthetic
aaatgaacaaaactagaggaatgacattacctgacttcaaattatactacagagctatagtaaccaaaacagcatggtacaggcat

SEQ ID NO: 32 - DNA, 69 bp, Artificial Sequence
  Synthetic
gtaatggaatggaatggaaaggaatcgaaacgaaaggaatggagacagatggaatggaatggaacagag

SEQ ID NO: 33 - DNA, 74 bp, Artificial Sequence
  Synthetic
atcgaatggaatcaacatcaaacggaaaaaaacggaattatcgaatggaatcgaagagaatcatcgaatggacc

SEQ ID NO: 34 - DNA, 57 bp, Artificial Sequence
  Synthetic
caatcagagcggacacaaacaaattgcatgggaagaatcaatatcgtgaaaatggcc

SEQ ID NO: 35 - DNA, 80 bp, Artificial Sequence
  Synthetic
agacctttctcagaagacacacaaattgccaacaggtatatgaaaaaatgttcaatatcactaatcatcagggcgatgcc

SEQ ID NO: 36 - DNA, 106 bp, Artificial Sequence
  Synthetic
catggaatcgaatggaattatcatcgaatggaatcgaatggtaccaacaccaaacggaaaaaaacggaattatcgaatggaatcgaagagaatcttcgaacggacc

SEQ ID NO: 37 - DNA, 86 bp, Artificial Sequence
  Synthetic
gaacgatttatcactgaaaattaatactcatgcaagtagtaaacgaatgtaatgaccatgataaggagacggacggtggtgatagt

SEQ ID NO: 38 - DNA, 80 bp, Artificial Sequence
  Synthetic
aaagatcaangnncaaaaatcagcagcatttctataaaccaacaatgtccaggctgagagngaaatcaagaaancaattc

SEQ ID NO: 39 - DNA, 121 bp, Artificial Sequence
  Synthetic
acacacataccaacagaacatgacaaaagaacaaaaccagccgcatgcatactcgatggagacaaaggtaacactgcagaatggtgaaggaagaacagtcattttaatgacagtgttggct

SEQ ID NO: 40 - DNA, 70 bp, Artificial Sequence
  Synthetic
aatggaatcaacatcaaacggaaaaaaacggaattatcgaatggaatcgaagagaatcatcgaatggacc

SEQ ID NO: 41 - DNA, 99 bp, Artificial Sequence
  Synthetic
atcaaaaggaacggaatggaatggaatggaatggaatggaatggaatggaatggaatgaaatcaacccgaatggaatggattggcatagagtggaatgg

SEQ ID NO: 42 - DNA, 90 bp, Artificial Sequence
  Synthetic
taaagaaaaacaaacaaacagaaatcaatgaaaatcccattcaaaggtcagcaacctcaaagactgaaggtagataagcccacaaggatg

SEQ ID NO: 43 - DNA, 55 bp, Artificial Sequence
  Synthetic
aaacggaaaaaaacggaattatcgaatggaatcgaatagaatcatcgaatggacc

SEQ ID NO: 44 - DNA, 96 bp, Artificial Sequence
  Synthetic
ggaatcaactcgattgcaatggaatgcaatggaaaggaatggaatgcaattaaagcgaatagaatggaatggaatggaatggaacggaatggaatg

SEQ ID NO: 45 - DNA, 83 bp, Artificial Sequence
  Synthetic
gaagaagaaaaaacatggatatacaatgtcaacagaaatcaaggagaaacggaatttcaccaatcaatttagtgatctgggtt

SEQ ID NO: 46 - DNA, 132 bp, Artificial Sequence
  Synthetic
tggaatcatctaatggaatggaatggaataatccatggactcgaatgcaatcatcataaaatggaatcgaatggaatcaacatcaaatggaatcaaatgggatcattgaacggaattgaatggaatcgtcat

SEQ ID NO: 47 - DNA, 105 bp, Artificial Sequence
  Synthetic
tgaacagagaattggacaaaacgcacaaagtaaagaaaaagaatgaagcaacaaaagcagagatttattgaaaacaaaagtacacaccacacagggtgggagtgg

SEQ ID NO: 48 - DNA, 67 bp, Artificial Sequence
  Synthetic
ggaatcaacatcaaacggaaaaaaacggaattatcgaatggaatcgaagagaatcatcgaatggacc

SEQ ID NO: 49 - DNA, 98 bp, Artificial Sequence
  Synthetic
aacacgactttgagaagagtaagtgattgttaattaaagcaagagaattattgatgtatcacagtcatgagaaatattggaaggaatatggtccatac

SEQ ID NO: 50 - DNA, 80 bp, Artificial Sequence
  Synthetic
tgaaaagaagaatgaccataagcaagcagatgaaaaacaaaacagaatttttacagacgtcttggactgatatcttgggc

SEQ ID NO: 51 - DNA, 80 bp, Artificial Sequence
  Synthetic
aatcaataaatgtaaaccagcatataaacagaaccaacgacaaaaaccacatgattatctcaatagatgcagaaaaggcc

SEQ ID NO: 52 - DNA, 95 bp, Artificial Sequence
  Synthetic
caacatcaaacggaatcaaacggaattatcgaatggaatcgaagagaatcatcgaatggactcgaatggaatcatctaatggaatggaatggaag

SEQ ID NO: 53 - DNA, 85 bp, Artificial Sequence
  Synthetic
aatggaagggaatggaatggaatcgaatcgaatggaacagaattcaatggaatggaatggaatggaatggaatcgaatggaatgg

SEQ ID NO: 54 - DNA, 90 bp, Artificial Sequence
  Synthetic
aaagacttaaacataagacctaaaaccataaaaaccacagaagaaaacataggcaatgccattcaggacataggcatgggcaaagacttc

SEQ ID NO: 55 - DNA, 80 bp, Artificial Sequence
  Synthetic
agacttgaaaagcacagacaacgaaagcaaaaatggacaaatggaatcacatcaagctaaaaggttttgcatggcaaagg

SEQ ID NO: 56 - DNA, 88 bp, Artificial Sequence
  Synthetic
agcaacttcagcaaagtctcaggatacaaaatcaatgtgcaaaaatcacaagcattcttatacaccaacaacagacaaacagagagcc

SEQ ID NO: 57 - DNA, 147 bp, Artificial Sequence
  Synthetic
tgaatgctatagagcagtaaaaacaaataaatgaactacattacagctacttacaaccatatgaaagaatataaccataacaatgatgagtggacaaaagctaagtgtgaaagaatgcatagtgctacagcagccaacatttacagc

SEQ ID NO: 58 - DNA, 100 bp, Artificial Sequence
  Synthetic
aacaaaattgaacaacatgcaaagaaacataaacgaagcaatgaaagtgtgcagatccactgaaatgaaagtgctgtccagagtgggagccagctcgaga

SEQ ID NO: 59 - DNA, 136 bp, Artificial Sequence
  Synthetic
tggaattatcgtcgaatagaatcgaatggtatcaacatcaaacggaaaaaaacggaattatcgaatggaatcgaagagaatcatcgaacggactcgaatggaatcatctaatggaatggaatggaataatccatgg

SEQ ID NO: 60 - DNA, 94 bp, Artificial Sequence
  Synthetic
agataagtggatgaacagatggacagatggatggatggatggatggatggatggatgcctggaagaaagaagaatggatagtaagctgggtata

SEQ ID NO: 61 - DNA, 55 bp, Artificial Sequence
  Synthetic
aatcaaagaattgaatcgaatggaatcatctaatgtactcgaatggaatcaccat

SEQ ID NO: 62 - DNA, 79 bp, Artificial Sequence
  Synthetic
aatggaatcgaacggaatcatcatcaaacggaaccgaatggaatcattgaatggaatcaaaggcaatcatggtcgaatg

SEQ ID NO: 63 - DNA, 82 bp, Artificial Sequence
  Synthetic
aggaatctataatacagctgtttatagccaagcactaaatcatatgatacagaaaacaaatgcagatggtttgaagggtggg

SEQ ID NO: 64 - DNA, 54 bp, Artificial Sequence
  Synthetic
aacggaaaaaaacggaattatcgaatggaatcgaagagaatcatcgaatggacc

SEQ ID NO: 65 - DNA, 124 bp, Artificial Sequence
  Synthetic
tgagaaaatgatggaaaagaggaataanacgaaacaaaaccacaggaacacaggtgcatgtgaatgtgcacagacaaagatacagggcggactgggaaggaagtttctgcaccagaatttgggg

SEQ ID NO: 66 - DNA, 79 bp, Artificial Sequence
  Synthetic
aatggaatcgaagagaatggaaacaaatggaatggaattgaatggaatggaattgaatggaatgggaaggaatggagtg

SEQ ID NO: 67 - DNA, 76 bp, Artificial Sequence
  Synthetic
aatgtcaagtggaatcgagtggaatcatcgaaagaaatcgaatggaatcgaagggaatcattggatgggctcaaat

SEQ ID NO: 68 - DNA, 84 bp, Artificial Sequence
  Synthetic
aaacaatggaagataatggaaagatatcgaatggaatagaatggaatggaatggactcaaatggaatggactttaatggaatgg

SEQ ID NO: 69 - DNA, 84 bp, Artificial Sequence
  Synthetic
gaacaatcaatggaagcagaaacaaataaaccaaggtgtgcatcaaggaatacattcacgcatgatggctgtatgagtaaaatg

SEQ ID NO: 70 - DNA, 85 bp, Artificial Sequence
  Synthetic
aaaccgaatggaatggaatggacgcaaaatgaatggaatggaagtcaatggactcgaaatgaatggaatggaatggaatggaatg

SEQ ID NO: 71 - DNA, 68 bp, Artificial Sequence
  Synthetic
aggatacaaaatcaaagtgcaaaaatcacaagcattcttatacaccaataacagacaaacagagagcc

SEQ ID NO: 72 - DNA, 77 bp, Artificial Sequence
  Synthetic
ggaatcgaatggaatcaacatcaaacggaaaaaaacagaattatcgtatggaatcgaatagaatcatcgaatggacc

SEQ ID NO: 73 - DNA, 96 bp, Artificial Sequence
  Synthetic
caacccgagtggaataaaatggaatggaatggaatgaaatggaatggatcggaatggaatccaatggaatcaactggaatggaatggaatggaatg

SEQ ID NO: 74 - DNA, 88 bp, Artificial Sequence
  Synthetic
tatcatcgaatggaatcgaatggaatcaacatcaaacggaaaaaaacggaattatcgaatggaatcgaagagaatcatcgaatggacc

SEQ ID NO: 75 - DNA, 88 bp, Artificial Sequence
  Synthetic
cggaataatcattgaacggaatcgaatggaatcatcatcggatggaaacgaatggaatcatcatcgaatggaaatgaaaggagtcatc

SEQ ID NO: 76 - DNA, 83 bp, Artificial Sequence
  Synthetic
caacacacagagattaaaacaaacaaacaaacaatccagccctgacatttatgagtttacagactggtggagaggcagagaag

SEQ ID NO: 77 - DNA, 107 bp, Artificial Sequence
  Synthetic
ggaatggaatgaacacgaatgtaatgcaacccaatagaatggaatcgaatggcatggaatataaagaaatggaatcgaagagaatggaaacaaatggaatggaattg

SEQ ID NO: 78 - DNA, 71 bp, Artificial Sequence
  Synthetic
cactacaaaccacgctcaaggcaataaaagaacacaaacaaatggaaaaacattccatgctcatggatggg

SEQ ID NO: 79 - DNA, 75 bp, Artificial Sequence
  Synthetic
aatcgaatggaattaacatcaaacggaaaaaaacggaattatcgaatggaatcgaagagaatcatcgaatggacc

SEQ ID NO: 80 - DNA, 82 bp, Artificial Sequence
  Synthetic
tggaaaagaatcaaattgaatggcatcgaacggaatgggatggaatggaatagacccagatgtaatggactcgaatggaatg

SEQ ID NO: 81 - DNA, 81 bp, Artificial Sequence
  Synthetic
aatcagtctagatcttaaaggaacaccagagggagtatttaaatgtgcccaataagcaagaattatggtgatgtggaagta

SEQ ID NO: 82 - DNA, 74 bp, Artificial Sequence
  Synthetic
ccataacacaattaaaaacaacctaaatgtctaatagaagaacactgttcagaccgggcatggtggcttatacc

SEQ ID NO: 83 - DNA, 95 bp, Artificial Sequence
  Synthetic
gactaatattcagaatatacaaggaactcaaacaactcaacagtagaaaaaaaaacctgaatagacatttctcaaaagaagacatacaaatggcc

SEQ ID NO: 84 - DNA, 98 bp, Artificial Sequence
  Synthetic
aacagaccataaataaacacagaagacacacgagtgtaaagtcagtgccccgctgcgaattaaatcggggtgatgtgatggcgagtgagtgggtagtt

SEQ ID NO: 85 - DNA, 138 bp, Artificial Sequence
  Synthetic
atcattgaatgcaatcacatggaatcatcacagaatggaatcgtacggaatcatcatcgaatggaattgaatggaatcatcaattggactcgaatggaaacatcaaatggaatcgattggaagtgtcgaatggactcg

SEQ ID NO: 86 - DNA, 67 bp, Artificial Sequence
  Synthetic
ggtccattcgatgattctcttcgattccattcgataattccgttttttcccgtttgatgttgattcc

SEQ ID NO: 87 - DNA, 91 bp, Artificial Sequence
  Synthetic
agcaacttcagtaaagtgtcaggatacaaaatcaatgtgcaaaaatcacaagcattcttatacatcaataacagacaaacagagagccaaa

SEQ ID NO: 88 - DNA, 88 bp, Artificial Sequence
  Synthetic
agcaacttcagcaaagtctcaggatacaaaatcaatgtgcaaaaatcacaagcattcctatacaccaacaacagacaaacagagagcc

SEQ ID NO: 89 - DNA, 86 bp, Artificial Sequence
  Synthetic
gaataatcattgaacggaatcgaatggaatcatcatcggatggaaacgaatggaatcatcatcgaatggaaatgaaaggagtcatc

SEQ ID NO: 90 - DNA, 66 bp, Artificial Sequence
  Synthetic
taatcatcttcgaattgaaaacaaagcaatcattaaatgtactctaacggaatcatcgaatggacc

SEQ ID NO: 91 - DNA, 77 bp, Artificial Sequence
  Synthetic
ggaatcgaatggaatcaacatcaaacggaaaaaaacggaattatcgaatggaatcgaagagaatcatcgaatggacc

SEQ ID NO: 92 - DNA, 113 bp, Artificial Sequence
  Synthetic
gatcagcttagaatacaatggaacagaacagattagaacaatgtgattttattaggggccacagcactgttgactcaagtacaagttctgactcatgtagaactaacactttt

SEQ ID NO: 93 - DNA, 86 bp, Artificial Sequence
  Synthetic
agagaaaagatgatcatgtaaccattgaaaagacaatgtacaaaactaatactaatcacacaggaccagaaagcaatttagaccat

SEQ ID NO: 94 - DNA, 79 bp, Artificial Sequence
  Synthetic
aatggaatcgaatggaatcaacatcaaacggaaaaaacggaattatcgaatggaatcaaagagaatcatcgaatggacc

SEQ ID NO: 95 - DNA, 96 bp, Artificial Sequence
  Synthetic
aatggaattatcatcgaatggaatcgaatggaatcaacatcaaacggaaaaaaacggaattatcgaatggaatcgaagagaatcatcgaatggacc

SEQ ID NO: 96 - DNA, 149 bp, Artificial Sequence
  Synthetic
gtcaacacaggaccaacataggaccaacacagggtcaacacaggaccaacataggaccaacacagggtcaacacaagaccaacatgggaccaacacagggtcaacataggaccaacatgggaccaacacagggtcaacacaggaccaac

SEQ ID NO: 97 - DNA, 109 bp, Artificial Sequence
  Synthetic
tatagttgaatgaacacacatacacacacacatgccacaaaacaaaaacaaagttatcctcacacacaggatagaaaccaaaccaaatcccaacacatggcaagatgat

SEQ ID NO: 98 - DNA, 79 bp, Artificial Sequence
  Synthetic
gaatcaactcgattgcaatcgaatggaatggaatggtattaacagaatagaatggaatggaatggaatggaacggaacg

SEQ ID NO: 99 - DNA, 86 bp, Artificial Sequence
  Synthetic
aatggaatggaataatcgacggacccgaatgcaatcatcatcgtacagaatcgaatggaatcatcgaatggactggaatggaatgg

SEQ ID NO: 100 - DNA, 106 bp, Artificial Sequence
  Synthetic
aatacaaaccactgctcaacgaaataaaagaggatacaaacaaatggaagaacattctatgctcatgggtaggatgaattcatatcgtgaaaatggccatactgcc

SEQ ID NO: 101 - DNA, 142 bp, Artificial Sequence
  Synthetic
aaacacgcaaacacacacacaagcacactaccacacaagcggacacacatgcaaacacgcgaacacacacacatatacacacaagcacattacaaaacacaagcaaacaccagcagacacacaaacacacaaacatacatgg

SEQ ID NO: 102 - DNA, 76 bp, Artificial Sequence
  Synthetic
aatcgaacggaatcaacatcaaacggaaaaaaaacggaattatcgaatggaatcgaagagaatcatcgaatggacc

SEQ ID NO: 103 - DNA, 88 bp, Artificial Sequence
  Synthetic
acacatttcaaggaaggaaacaagaacagacagaaacacaacatacttcatgaaaccacattttagcatcctggccgagtattcatca

SEQ ID NO: 104 - DNA, 67 bp, Artificial Sequence
  Synthetic
ggatacaaaatcaatgtacaaaaatcacaagcattcttatacaccaataacagacaaacagagagcc

SEQ ID NO: 105 - DNA, 81 bp, Artificial Sequence
  Synthetic
taattgattcgaatggaatggaatagaatggaattgaatggaatggaccataatggattggactttaatagaaagggcatg

SEQ ID NO: 106 - DNA, 88 bp, Artificial Sequence
  Synthetic
agcaacttcagcaaagtctcaggatacaaaatcaatgtacaaaagtcacaagcattcttatacaccaacaaaagacaaacagagagcc

SEQ ID NO: 107 - DNA, 68 bp, Artificial Sequence
  Synthetic
acatcaaacggaaaaaaaaaacaaaacggaattatcgaatggaatcgaagagaatcatcgaatggacc

SEQ ID NO: 108 - DNA, 92 bp, Artificial Sequence
  Synthetic
acatctcacttttagtaatgaacagatcattcagacagaaaattagcaaagaaacatcagagttaaactacactctaaaccaaatggaccta

SEQ ID NO: 109 - DNA, 88 bp, Artificial Sequence
  Synthetic
gaagaaagcattcattcaagacatctaactcgttgatataatgcatacagttcaaaatgattacactatcattacatctagggctttc

SEQ ID NO: 110 - DNA, 83 bp, Artificial Sequence
  Synthetic
gcaaaagaaacaatcagtagagtaaacagacaactcatagaatgcaagaaaatcatcgcaatctgtacatccaacaaagggct

SEQ ID NO: 111 - DNA, 62 bp, Artificial Sequence
  Synthetic
acacacacattcaaagcagcaatatttacaacagccaaaaggtggaaacaattgagcaattg

SEQ ID NO: 112 - DNA, 87 bp, Artificial Sequence
  Synthetic
atcatcgaatagaatcgaatggtatcaacaccaaacggaaaaaaacggaattatcgaatggaatcgaagagaatcttcgaacggacc

SEQ ID NO: 113 - DNA, 95 bp, Artificial Sequence
  Synthetic
tgaaaatacaaatgaccatgcaagtaattccgcagggagagagcggatatgaacaaacagaagaaatcagatgggatagtgctggcgggaagtca

SEQ ID NO: 114 - DNA, 92 bp, Artificial Sequence
  Synthetic
aatcgaaaggaatgtcatcgaatggaatggactcaaatggaatagaatcggatggaatggcatcgaatggaatggaatggaattggatggac

SEQ ID NO: 115 - DNA, 138 bp, Artificial Sequence
  Synthetic
aacatgaacagtggaacaatcagtgaaccaatacaagggttaaataagctagcaattaaaagctgtatcactggtctaaagatagaagatcaagtagaaaatcagcgcaagaggaaagatatacgaaaactaatggcc

SEQ ID NO: 116 - DNA, 123 bp, Artificial Sequence
  Synthetic
cgaatggaatcattatggaatggaatgaaatggaataatcaaatggaattgaatggaatcatcgaatggaatcgaacaaaatcctctttgaatggaataagatggaatcaccaaatggaattg

SEQ ID NO: 117 - DNA, 100 bp, Artificial Sequence
  Synthetic
aaacggaatcaaacggaattatcgaatggaatcgaagagaatcatcgaacggactcgaatggaatcatctaatggaatggaatggaagaatccatggact

SEQ ID NO: 118 - DNA, 96 bp, Artificial Sequence
  Synthetic
gctagttcaacatatgcaaatcaataaacgtaatccatcacataaacagaaccaatgacaaaaaccacgattatctcaatagatgcagaaaaggcc

SEQ ID NO: 119 - DNA, 61 bp, Artificial Sequence
  Synthetic
accaatcaagaaaacaatgcaacccacagagaatggacaaaagcaaggcaggacaatggct

SEQ ID NO: 120 - DNA, 74 bp, Artificial Sequence
  Synthetic
atcgaatggaatcaacatcagacggaaaaaaacggaattatcaaatggaatcgaagagaatcatcgaatggacc

SEQ ID NO: 121 - DNA, 101 bp, Artificial Sequence
  Synthetic
atggaatcaacatcaaacggaaaaaaaaacggaattatcgaatggaatcgaagagaatcatcgaatggaccagaatggaatcatctaatggaatggaatgg

SEQ ID NO: 122 - DNA, 88 bp, Artificial Sequence
  Synthetic
aatggaatcatcatcgaatggaatcgaatggaatcatggaatggaatcaaatggaatcaaatggaatcgaatggaatggaatggaatg

SEQ ID NO: 123 - DNA, 87 bp, Artificial Sequence
  Synthetic
aacggaatcaaacggaattaccgaatggaatcgaatagaatcatcgaacggactcgaatggaatcatctaatggaatggaatggaag

SEQ ID NO: 124 - DNA, 97 bp, Artificial Sequence
  Synthetic
aaacggaatcaaacggaattatcgaatggaatcgaaaagaatcatcgaacggactcgaatggaatcatctaatggaatggaatggaagaatccatgg

SEQ ID NO: 125 - DNA, 88 bp, Artificial Sequence
  Synthetic
agcaacttcagcaaagtctcaggatacaaaatcaatgtacaaaaatcacaagcattcttatacaccaataacagacaaacagagagcc

SEQ ID NO: 126 - DNA, 71 bp, Artificial Sequence
  Synthetic
gaatgatacggantannnngnaatggaacgaaatgaaatggaatggaatggaatggaatggaatggaatgg

SEQ ID NO: 127 - DNA, 89 bp, Artificial Sequence
  Synthetic
aatggactcgaatggattaatcattgaacggaatcgaatggaatcatcatcggatggtaatgaatggaatcatcatcgaatggaatcgg

SEQ ID NO: 128 - DNA, 101 bp, Artificial Sequence
  Synthetic
gaatggaatcgaaaggaatgtcatcgaatggaatggaatggaacggaatggaatcgaatggaatggactcgaatggaatagaatcgaatgcaatggcatcg

SEQ ID NO: 129 - DNA, 69 bp, Artificial Sequence
  Synthetic
gaatagaatagaatggaatcatcgaatggaatcgaatggaatcatcatgatatggaattgagtggaatc

SEQ ID NO: 130 - DNA, 107 bp, Artificial Sequence
  Synthetic
taagccgataagcaacttcagcaaagtctcaggagacaaaatcaatgtgcaaaaaatcacaagcattcttatacactaataacagacaaacagagagccaaatcatg

SEQ ID NO: 131 - DNA, 88 bp, Artificial Sequence
  Synthetic
agcaacttcagcaaagtctcaggatacaaaatcaatgtgcaaaaatcaaaagcattcttatgcaccaataacagacacagagccaaat

SEQ ID NO: 132 - DNA, 81 bp, Artificial Sequence
  Synthetic
aggaaagttttcaatatgagaaagatacaaaccaacagaataagcaaactggataaacagaaaatacagagagagccaagg

SEQ ID NO: 133 - DNA, 79 bp, Artificial Sequence
  Synthetic
aatggaatggaacgcaattgaatggaatggaatggaacggaatcaacctgagtcaaatggaatggaatggaatggaatg

SEQ ID NO: 134 - DNA, 96 bp, Artificial Sequence
  Synthetic
aggaaaatgcaaatcagaacgactataacacaccatctcaaactcgttaggatggctattatcaaaaagtcaagagataacaaatgtgggcaaggg

SEQ ID NO: 135 - DNA, 82 bp, Artificial Sequence
  Synthetic
ggaacgaaatcgaatggaacggaatagaatagactcgaatgtcatggattgctatgtaattgattggaatggaatggaatcg

SEQ ID NO: 136 - DNA, 96 bp, Artificial Sequence
  Synthetic
gaattgaaaggaatgtattggaataaaatggaatcgaataggttgaaataccataggttcgaattgaatggaatgggagggacaccaatggaattg

SEQ ID NO: 137 - DNA, 106 bp, Artificial Sequence
  Synthetic
aacaaaacaaaaacccaactcaataacaagaagacaaacaacccaatttaaaatgagcaaagaacttgataaacatgtctccaaagaagatacggccaaagagcac

SEQ ID NO: 138 - DNA, 83 bp, Artificial Sequence
  Synthetic
atggttaaaactcaacaatgaaaacacaaacagcgcaatttaaaaatgggcaaaatgacaggccagacccagtggctcatgcg

SEQ ID NO: 139 - DNA, 89 bp, Artificial Sequence
  Synthetic
aagcaacttcagcaaagtctcgggatacaaaatcaatgtgcaaaaatcacaagcattcttatacaccactaacagacaaatggagagtc

SEQ ID NO: 140 - DNA, 113 bp, Artificial Sequence
  Synthetic
gaatggaatcaacatcaaacggaaaaaaacggaattatcgaatggaatcgaagagaatcatcgaatggaccagaatggaatcatctaatggaatggaatggaataatccatgg

SEQ ID NO: 141 - DNA, 83 bp, Artificial Sequence
  Synthetic
tagaaggaatttgatacatgctcagaaatacaggcaaaggaagtaggtgcctgccagtgaacacaggggaactatggctccta

SEQ ID NO: 142 - DNA, 77 bp, Artificial Sequence; identical to SEQ ID NO: 91
  Synthetic

SEQ ID NO: 143 - DNA, 95 bp, Artificial Sequence
  Synthetic
aactaagacaacagattgatttacactactattttcacacagccaaaaatatcactatggcaatcgtcaaaaggtcaattcaaagatgggacagt

SEQ ID NO: 144 - DNA, 77 bp, Artificial Sequence
  Synthetic
aaaagcaattggactgattttaaatatacgtggcaacaaggataaactgctaatgatgggtttgcaaatacagatcg

SEQ ID NO: 145 - DNA, 70 bp, Artificial Sequence
  Synthetic
aatggaatcaacatcgaacggaaaaaaacggaattatcgaatggaatcgaagagaatcatcgaatggacc

SEQ ID NO: 146 - DNA, 108 bp, Artificial Sequence
  Synthetic
tgcaagataacacattttagttgacaccattgaaaacagttttaaccaagaatattagaaccaatgaagcagagaaatcaaaagggtggatggaactgccaaaggatg

SEQ ID NO: 147 - DNA, 97 bp, Artificial Sequence
  Synthetic
tagaacagaattgaatggaatggcatcaaatggaatggaaacgaaaggaatggaattgaatggactcaaatgttatggaatcaaagggaatggactc

SEQ ID NO: 148 - DNA, 93 bp, Artificial Sequence
  Synthetic
aagagaatcatcgaatggaatcgaatggaatcaacatcaaacggaaaaaaacggaattatcgaatggaatcgaagagaatcatcgaatggacc

SEQ ID NO: 149 - DNA, 64 bp, Artificial Sequence
  Synthetic
atcaacatcaaacggaaaaaaacggaattatcgaatggaatcgaagagaatcatcgaatggacc

SEQ ID NO: 150 - DNA, 66 bp, Artificial Sequence
  Synthetic
gaatcaacatcaaacggaaaaaaaccgaattatcgaatggaatcgaagagaatcatcgaatggacc

SEQ ID NO: 151 - DNA, 106 bp, Artificial Sequence
  Synthetic
atcaacatcaaacggaatcaaacggaattatcgaatggaatcgaagagaatcatcaaatggactcgaatggaatcatctaatggaatggaatggaagaatccatgg

SEQ ID NO: 152 - DNA, 99 bp, Artificial Sequence
  Synthetic
aaacagttcaaaaattattgcaacaaaatgagagagatgagtttatcttgcaaactaatggatggtagcagtgacagtggcaaaacgtggtttgattct

SEQ ID NO: 153 - DNA, 89 bp, Artificial Sequence
  Synthetic
atcgaatggaatcattgaatggaaaggaatggaatcatcatggaatggaaacgaatggaatcactgaatggactcgaatgggatcatca

SEQ ID NO: 154 - DNA, 90 bp, Artificial Sequence
  Synthetic
attcagcctttaaaaaaagaagacagtcctgtcatttgtgacaatatgaatgaaacagacatcacattaaatgaaatgagccaggcgcag

SEQ ID NO: 155 - DNA, 83 bp, Artificial Sequence
  Synthetic
aggagaatagcagtagaatgacaaaattagattttcacatgaaacttgatgacagtgtaggaaatggactgaaaggacaagac

SEQ ID NO: 156 - DNA, 87 bp, Artificial Sequence
  Synthetic
aacccacaaagacaacagaagaaaagacaacagtagacaaggatgtcaaccacattttggaagagacaagtaatcaaacacatggca

SEQ ID NO: 157 - DNA, 83 bp, Artificial Sequence
  Synthetic
gaaaatgaacaatatgaacaaacaaacaaaattactacccttacgaaagtacgtgcattctagtatggtgacaaaaaggaaag

SEQ ID NO: 158 - DNA, 157 bp, Artificial Sequence
  Synthetic
aacatcaaacggaatcaaacggaattatcgaatggaatcgaagagaatcatcgaacggactcgaatggaatcatctaatggaatggaatggaagaatccatggactcgaatgcaatcatcatcgaatgaaatcgaatggaatcatcgaatggactcg

SEQ ID NO: 159 - DNA, 75 bp, Artificial Sequence
  Synthetic
accaacataagacaaagaaacatccagcagctgcctatggcaaaagattacaatgtgtcaaacaagagggcaatg

SEQ ID NO: 160 - DNA, 72 bp, Artificial Sequence
  Synthetic
atggaattcaatggaatggacatgantgnaatgnacttcaatggaatggnatcnaatggaatgnaattcant

SEQ ID NO: 161 - DNA, 79 bp, Artificial Sequence
  Synthetic
tatgactttcacaaattacagaaaaagacacccatttgacaagggaactgaaggtggtgaagacatactggcaggctac

SEQ ID NO: 162 - DNA, 85 bp, Artificial Sequence
  Synthetic
aatggaaaggaatcgaatggaagggaatgaaattgaatcaacaggaatggaagggaatagaatagacggcaatggaatggactcg

SEQ ID NO: 163 - DNA, 113 bp, Artificial Sequence
  Synthetic
agcctatcaaaaagtgggctaagaatatgaatacacaattctcaaaagaagatatacaaatgggcaacaaacatatgaaaacatactcaacatcactaatgatcagggaaatg

SEQ ID NO: 164 - DNA, 88 bp, Artificial Sequence
  Synthetic
agcaacttcagcaaagtatcaggatacaaaatcaatgtacaaaaatcccaagcattcttatacaccaacaacagacaaacagagagcc

SEQ ID NO: 165 - DNA, 87 bp, Artificial Sequence
  Synthetic
aaagacaatatacaaatggccaataagcacatgaaaagacgctcaacatccttagtcgttaaggcaatgcaaatcaaaaccacaatg

SEQ ID NO: 166 - DNA, 88 bp, Artificial Sequence
  Synthetic
agcaacttcagcaaagtctcaggatacaaaatcgatgtgcaaaaatcacaagcattcttatacaccaacaacagataaacagagagcc

SEQ ID NO: 167 - DNA, 106 bp, Artificial Sequence
  Synthetic
aacggaaaaaaaacggaattatcgaatggaatcgaagagaatcatcgaatggaccagaatggaatcatctaatggaatggaatggaataatccatggactcgaatg

SEQ ID NO: 168 - DNA, 70 bp, Artificial Sequence
  Synthetic
aacagcaatagacacaaagtcagcacttacagtacaaaaactaatggcaaaagcacatgaagtgggacat

SEQ ID NO: 169 - DNA, 102 bp, Artificial Sequence
  Synthetic
ggaatcaaacggaattatcgaatggaatcgaagagaatcatagaacggactcaaatggaatcatctaatggaatggaatgggagaatccatggactcgaatg

SEQ ID NO: 170 - DNA, 70 bp, Artificial Sequence
  Synthetic
aatggaatcaatatcaaacggaaaaaaacggaattatcgaatggaatcgaagagaatcatcgaatggacc

SEQ ID NO: 171 - DNA, 87 bp, Artificial Sequence
  Synthetic
aaaatgatcatgagaaaattcagcaacaaaaccatgaaattgcaaagatattacttttgggatggaacagagctggaaggcaaagag

SEQ ID NO: 172 - DNA, 96 bp, Artificial Sequence
  Synthetic
aacggaatcaaacggaattatcgaatggaatcgaaaagaatcatcgaacggactcgaatggaatcatctaatggaatggaatggaagaatccatgg

SEQ ID NO: 173 - DNA, 90 bp, Artificial Sequence
  Synthetic
aaacggaattatcgaanggaatcaaagagaatcatcgaannnnnacgaatggaatcatataatggaatggaatggaataatccatggacc

SEQ ID NO: 174 - DNA, 85 bp, Artificial Sequence
  Synthetic
aatggaatcgaatggattgatatcaaatggaatggaatggaagggaatggaatggaatggaattgaaccaaatgtaatggatttg

SEQ ID NO: 175 - DNA, 89 bp, Artificial Sequence
  Synthetic
taaaagacggaacagatagaaagcagaaaggaaaggtgaattgcattaccactattcatactgccacacacatgacattaggccaagtc

SEQ ID NO: 176 - DNA, 62 bp, Artificial Sequence
  Synthetic
acaaacaatccaattcgaaaatgggcaagatatttcaccaaagacatgagctgatatttcac

SEQ ID NO: 177 - DNA, 79 bp, Artificial Sequence
  Synthetic
aatggaatcgaatggaacaatcaaatggactccaatggagtcatctaatggaatcgagtggaatcatcgaatggactcg

SEQ ID NO: 178 - DNA, 102 bp, Artificial Sequence
  Synthetic
taacacataaacaaacacagagacaaaatctccgagatgttaatctgctccagcaatacagaacaatttctattaccaacagaatgcttaatttttctgcct

SEQ ID NO: 179 - DNA, 77 bp, Artificial Sequence
  Synthetic
ggaatcgaatggaatcaacatcaaacggaaaaaaacggaattatcgaatggaatcaaagagaatcatcgaatggacc

SEQ ID NO: 180 - DNA, 51 bp, Artificial Sequence
  Synthetic
agaatggaaaggaatcgaaacgaaaggaatggagacagatggaatggaatg

SEQ ID NO: 181 - DNA, 71 bp, Artificial Sequence
  Synthetic
gaatggaatggaaaggaatcgaaacgaaaggaatggagacagatggaatggaatggaacagagagcaatgg

SEQ ID NO: 182 - DNA, 89 bp, Artificial Sequence
  Synthetic
gaatcatcataaaatggaatcgaatggaatcaacatcaaatggaatcaaatggtctcgaatggaatcatcttcaaatggaatggaatgg

SEQ ID NO: 183 - DNA, 127 bp, Artificial Sequence
  Synthetic
aacaacaatgacaaacaaacaacaacgacaaagacatttatttggttcacaaatctccagggtgtacaagaagcatggtgccagcatctgctcagcttctgatgagggctctgggaagcttttactc

SEQ ID NO: 184 - DNA, 96 bp, Artificial Sequence
  Synthetic
aacggactcgaacggaatataatggaatggaatggattcgaaaggaatggaatggaatggacaggaaaagaattgaatgggattggaatggaatcg

SEQ ID NO: 185 - DNA, 85 bp, Artificial Sequence
  Synthetic
aggaaataaaagaagacacaaacaaatggaagaacattccatgcttatggatagggagaatcagtatcgtgaaaatggccatact

SEQ ID NO: 186 - DNA, 112 bp, Artificial Sequence
  Synthetic
aacatcaaacgaaatcaaacggaattatcaaattgaatcgaagagaatcatcgaattgccacgaatgcaatcatctaatggtatggaatggaataatccatggacccagatg

SEQ ID NO: 187 - DNA, 94 bp, Artificial Sequence
  Synthetic
agaaattaacagcaaaagaaggatgcagtgcaactcaggacaacacatacaattcaagcaacaaatgtatagtggctgggcaccaaggatacag

SEQ ID NO: 188 - DNA, 82 bp, Artificial Sequence
  Synthetic
gcaataaaatcgactcagatagagaagaatgcaatggaatggaatggaatggaatggaatgggatggaatggtatggaatgg

SEQ ID NO: 189 - DNA, 82 bp, Artificial Sequence
  Synthetic
ccacataaaacaaaactacaagacaatgataaagttcacaacattaacacaatcagtaatggaaaagcctagtcaatggcag

SEQ ID NO: 190 - DNA, 91 bp, Artificial Sequence
  Synthetic
ggacaacatacacaaatcagtcaagatacatcatttcaacagaatgaaagacaaaaaccatttgatcacttcaatcgatgatgaaaaagca

SEQ ID NO: 191 - DNA, 72 bp, Artificial Sequence
  Synthetic
gaaatcatcatcaaacggaatcgaatggaatcattgaatggaatggaatggaatcatcatggaatggaaacg

SEQ ID NO: 192 - DNA, 157 bp, Artificial Sequence
  Synthetic
tggaatggantggaatgnaatcnaatcnnntggtaatgaatcaaatggaatcaaatcgaatggnaataatggaatcnannggaaacgaatggnatcgaattgcactgattctactgacttcgaggaaaatgaaatgaaatgcggtgaagtggaatgg

SEQ ID NO: 193 - DNA, 88 bp, Artificial Sequence
  Synthetic
agcaacttcagcaaagtctcaggatacaaaatcaatgggaaaaaatcacaagcattcctatacatcaataacagacaaacagagagcc

SEQ ID NO: 194 - DNA, 71 bp, Artificial Sequence
  Synthetic
gaatgttatgaaatcaactcgaacggaatgcaatagaatggaatggaatggaatggaatggaatggaatgg

SEQ ID NO: 195 - DNA, 102 bp, Artificial Sequence
  Synthetic
agtagaattgcaattgcaaatttcacacatatactcacacacaagtacacacatccacttttacaactaaaaaaactagcacccaggacaggtgcagtggct

SEQ ID NO: 196 - DNA, 68 bp, Artificial Sequence
  Synthetic
ggaatcaacatcaaacggaaaaaaaacggaattatcgaatggaatcgaagagaatcatcgaatggacc

SEQ ID NO: 197 - DNA, 69 bp, Artificial Sequence
  Synthetic
ggaataatcatcatcaaacagaaccaaatggaatcattgaatggaatcaaaggcaatcatggtcgaatg

SEQ ID NO: 198 - DNA, 101 bp, Artificial Sequence
  Synthetic
actcaggaaaaataacgaatccaactcacaggagaaagaagtacaaaccagaaaccaatttcaaattacaaggaccagaatactcatgttggctggccagt

SEQ ID NO: 199 - DNA, 83 bp, Artificial Sequence
  Synthetic
aaacgcacaaacaaagcaaggaaagaatgaagcaacaaaagcagagatttattgaaaatgaaaaatacactccacagggtggg

SEQ ID NO: 200 - DNA, 108 bp, Artificial Sequence
  Synthetic
gcatagaatcgaatggaattatcattgaatggaatcgaatggaatcaacatcaaacggaaaaaaacggaattatcgaatggaatcgaagagaatcatcgaatggaccc

SEQ ID NO: 201 - DNA, 82 bp, Artificial Sequence
  Synthetic
aatggaatcgaanagaatcatcgaacggactcgaatggaatcatctaatggaatggaatggaataatccatggacccgaatg

SEQ ID NO: 202 - DNA, 99 bp, Artificial Sequence
  Synthetic
aaatgaatcgaatggaattgaatggaatcaaatagaacaaatggaatcgaaatgaatcaaatggaatcgaatcgaatggaattgaatggcatggaattg

SEQ ID NO: 203 - DNA, 131 bp, Artificial Sequence
  Synthetic
ntcacaatcacacaacacattgcacatgnnnannatgcactcacaatacacacacaacacatacacaacacacatgcaatacaacacaaaacgcaacacaacatatacacnacacacagcacacanatgcc

SEQ ID NO: 204 - DNA, 74 bp, Artificial Sequence
  Synthetic
gaatggaatcaaatcgaatgaaatggaatggaatagaaaggaatggaatgaaatggaatggaaaggattcgaat

SEQ ID NO: 205 - DNA, 87 bp, Artificial Sequence
  Synthetic
aaagacttaaacgttagacctaaaaccataaaaaccctagaggaaaacctaggcattaccattcaggacttaggcatgggcaaggac

SEQ ID NO: 206 - DNA, 86 bp, Artificial Sequence
  Synthetic
gtttacagtcaagtgtacaaacagaatataagcaaacaaaagagaacatatacttacaaactatgctaagtgccatgaaggaaaag

SEQ ID NO: 207 - DNA, 108 bp, Artificial Sequence
  Synthetic
aaagtccaaagatgaacaaaatatccagaaggaaaacaaatgcacttggggagtgggaaagaaaaccaagactgagcaatgcgtcaagctcagacgtgcctcactacg

SEQ ID NO: 208 - DNA, 97 bp, Artificial Sequence
  Synthetic
aaacggaatcaaacggaattatcgaatggagtcgaaaagaatcatcgaacggactcgaatggaatcatctaatggaatggaatggaagaatccatgg

SEQ ID NO: 209 - DNA, 94 bp, Artificial Sequence
  Synthetic
aattgattcgaaattaatggaattgaatggaatgcaatcaaatggaatggaatgtaatgcaatggaatgtaatagaatggaaagcaatggaatg

SEQ ID NO: 210 - DNA, 79 bp, Artificial Sequence
  Synthetic
tacagaacacatgactcaacaacagcagaaagcatattcttttcaaatgcacatgaaacattatcatgatggaccaaat

SEQ ID NO: 211 - DNA, 75 bp, Artificial Sequence
  Synthetic
taagacacatagaaaacataaagcaaaatggcagatgtaaatgcaacctatcaatcaaaacattacgaatggctt

SEQ ID NO: 212 - DNA, 65 bp, Artificial Sequence
  Synthetic
ggaacaaaatgaaatcgaacggtaggaatcatacagaacagaaagaaatggaacggaatggaatg

SEQ ID NO: 213 - DNA, 68 bp, Artificial Sequence
  Synthetic
aacggaaaaaacggaattatcgaatggaatcgaagagaatcatcgaatggaatcgaatggagtcatcg

SEQ ID NO: 214 - DNA, 93 bp, Artificial Sequence
  Synthetic
aacatacgaaaatcaataaacgtaatccagcatataaacagaaccaaagacaaaaaccacatgattatctcaatagatgcagaaaaggccttt

SEQ ID NO: 215 - DNA, 75 bp, Artificial Sequence
  Synthetic
aatcgaacggaatcaacatcaaacggaaaaaaacggaattatcgaatggaatcgaagagaatcatcgaatggacc

SEQ ID NO: 216 - DNA, 87 bp, Artificial Sequence
  Synthetic
agaatggaatgcaatagaatggaatgcaatggaatggagtcatccgtaatggaatggaaaggaatgcaatggaatggaatggaatgg

SEQ ID NO: 217 - DNA, 98 bp, Artificial Sequence
  Synthetic
ggaataaaacggactcaatagtaatggattgcaatgtaattgattcgatttcgaatggaatcgcatggaatgtaatggaatggaatggaatggaaggc

SEQ ID NO: 218 - DNA, 88 bp, Artificial Sequence
  Synthetic
agcaacttcagcaaagtctcaggatacaaaatcaatgtacaaaaatcacaagcattcttatacaccaacaacagacaaacagagagcc

SEQ ID NO: 219 - DNA, 68 bp, Artificial Sequence
  Synthetic
taagcagagaaaatatcaacacgaaaataatgcaaggagaaaaatacagaacaatccaaaatgtggcc

SEQ ID NO: 220 - DNA, 70 bp, Artificial Sequence
  Synthetic
aatggaatcaacatcaaacggaaaaaaacggaattatcgtatggaatcgaaaagaattatcgaatggacc

SEQ ID NO: 221 - DNA, 57 bp, Artificial Sequence
  Synthetic
tcaaacggaaaaaaacggaattatcgaatggaatcgaagagaatcatcgaatggacc

SEQ ID NO: 222 - DNA, 85 bp, Artificial Sequence
  Synthetic
aacttcagcaaattctcaggatacaaaatcaatgtgcaaaaaccacaagcattcctatacaccaataatagacagtgagccaaat

SEQ ID NO: 223 - DNA, 111 bp, Artificial Sequence
  Synthetic
acatcaaacggaatcaaacggaattatcgaatggaatcgaaaagaatcatcgaacggactcgaatggaatcatctaatggaatggaatggaagaatccatggactcgaatg

SEQ ID NO: 224 - DNA, 76 bp, Artificial Sequence
  Synthetic
aatggaatcgaatgcaatcatcgaacggaatcgaatggcatcaccgaatggaatggaatggaatggaatggaatgg

SEQ ID NO: 225 - DNA, 67 bp, Artificial Sequence
  Synthetic
aatccagcatataaacagaaccaaagacaaaaaccacatgattatctcaatagatgcagaaaaggcc

SEQ ID NO: 226 - DNA, 131 bp, Artificial Sequence
  Synthetic
tgactaaacagagttgaacaagaacaaaaagcaaatttgcagaaatgaaatacatactaattgaaagtccatggacaggctcaacagatgatatagatacagctaaagagataattagtgaaatggatcag

SEQ ID NO: 227 - DNA, 75 bp, Artificial Sequence
  Synthetic
gatcatcagagaaacagagaaatgcaaattaaaaccacaatgagatactatctccacacaagtcagaatggctat

SEQ ID NO: 228 - DNA, 80 bp, Artificial Sequence
  Synthetic
aggatacaaaatcaatgtacaaaaatcacaaacattcttatacaccaacaacagacaaacagagagccaaatcatgggtg

SEQ ID NO: 229 - DNA, 99 bp, Artificial Sequence
  Synthetic
taagcaacttcagcaaagtctcaggatacaaaatcaatgtacaaaaatcacaagcattcttatacaccaacaacagacaaacaagagtgccaaatcatg

SEQ ID NO: 230 - DNA, 87 bp, Artificial Sequence
  Synthetic
agaattgattgaatccaagtggaattgaatggaatggaatggattagaaaggaatggaatggattggaatggattggaatggaaagg

SEQ ID NO: 231 - DNA, 76 bp, Artificial Sequence
  Synthetic
aatggaatgcaatcgaatggaatggaatcgaacggaatggaataaaatggaagaaaactggcaagaaatggaatcg

SEQ ID NO: 232 - DNA, 96 bp, Artificial Sequence
  Synthetic
aactgcatcaactaacaggcaaaataaccagctaatatcataatgacaggattaaattcacaaatgacaatattaaccgtaaatgtaaatgggcta

SEQ ID NO: 233 - DNA, 102 bp, Artificial Sequence
  Synthetic
tacaaagaactcaaacaaatcagcaagaacaaaaacaatcccaacaaaatgttggacaaagacatgaatagacaattctcgaaagaagatgtacaaatggct

SEQ ID NO: 234 - DNA, 99 bp, Artificial Sequence
  Synthetic
agagaaatgcaaatcaaaaccacaatggaataccatctcacgccagtcagaatggcaattattaaaaaatcacaacaattaatgatggcaaggctgtgg

SEQ ID NO: 235 - DNA, 108 bp, Artificial Sequence
  Synthetic
gtaaacaaacaatcaagcaagtaagaacagaaataacagcatttggcttttgagttaatgacaagaacactcggcatgggagcctgggtgagcaaatcacagatcttc

SEQ ID NO: 236 - DNA, 91 bp, Artificial Sequence
  Synthetic
gaatcaacccgagcggaaaggaatggaatggaatggaatcaacacgaatggaatggaacggaatggaatgggatgggatgaaatggaatgg

SEQ ID NO: 237 - DNA, 88 bp, Artificial Sequence
  Synthetic
agcaacttcagcaaagtctcaggagacaaaatcaatgtacaaaaatcacaagcattcttatacaccaataacagacaaacagagagcc

SEQ ID NO: 238 - DNA, 71 bp, Artificial Sequence
  Synthetic
aagaaatggaatcgaagagaatggaaacaaacggaatggaattgaatggaatggaattgaatggaatggga

SEQ ID NO: 239 - DNA, 101 bp, Artificial Sequence
  Synthetic
gacatgcaaacacaacacacagcacacatggaacatgcatcagacatgcaaacacaacacacataccacacatggcatatgcatcagacgtgcctcactac

SEQ ID NO: 240 - DNA, 91 bp, Artificial Sequence
  Synthetic
tacagataagaaaattgagactcaagagtattacataaattgtttcagctaccacagcaaaaaatggtatggttgggaatcaagctcaggg

SEQ ID NO: 241 - DNA, 135 bp, Artificial Sequence
  Synthetic
aaaggaatgcactcgaatggaatggacttgaatggaatgtctccgaatggaacagactcgtatgaaatggaatcgaatggaatggaatcaaatggaattgatttgagtgaaatggaatcaaatggaatggcaacg

SEQ ID NO: 242 - DNA, 95 bp, Artificial Sequence
  Synthetic
tgaaacaaatgataatgaaaatacaacataccaaacatacgagatacagtaaaagcagtactaagatgcaagtatatattgctacaagtgcctac

SEQ ID NO: 243 - DNA, 89 bp, Artificial Sequence
  Synthetic
ggaacaaaatgaaatcgaacggtaggaatcgtacagaacggaaagaaatggaacggaatggaatgcactcgaatggaaaggagtccaat

SEQ ID NO: 244 - DNA, 95 bp, Artificial Sequence
  Synthetic
aaattgattgaaatcatcataaaatggaatcgaagggaatcaacatcaaatggaatcaaatggaatcattgaacggaattgaatggaatcgtcat

SEQ ID NO: 245 - DNA, 96 bp, Artificial Sequence
  Synthetic
agaaaggattcgaatggaatgaaaaagaattgaatggaatagaacagaatggaatcaaatcgaatgaaatggaatggaatagaaaggaatggaatg

SEQ ID NO: 246 - DNA, 84 bp, Artificial Sequence
  Synthetic
agaatggaaagcaatagaatggaacgcactggattcgagtgcaatggaatcaattggaatggaatcgaatggaatggattggca

SEQ ID NO: 247 - DNA, 103 bp, Artificial Sequence
  Synthetic
aacaccaaacggaaaaaaacggaattatcgaatggaatcgaagagaatcttcgaacggacccgaatgggatcatctaatggaatggaatggaataatccatgg

SEQ ID NO: 248 - DNA, 94 bp, Artificial Sequence
  Synthetic
aatggagactaatgtaatagaatcaaatggaatggcatcgaatggaatggactggaatggaatgtgcatgaatggaatggaatcgaatggattg

SEQ ID NO: 249 - DNA, 84 bp, Artificial Sequence
  Synthetic
tgggatatgggtgaaagaacaagtttgcagaaaagatacagtgaattatggaccatgagttcgggaaagaagggtaggactgcg

SEQ ID NO: 250 - DNA, 151 bp, Artificial Sequence
  Synthetic
aaatcgaatggaacgcaatagaatagactcgaatgtaatggattgctatgtaattgattcgaatggaatggaatcgactggaatgcaatccaatggaatggaatgcaatgcaatggaatggaatcgaacggaatgcagtggaagggaatgg

SEQ ID NO: 251 - DNA, 107 bp, Artificial Sequence
  Synthetic
aatcaacaaggaactgaaacaagtaaacaagaaaacaaataacaccataaaacatgggcaaaggacataaacagacatttttcaaaaaagacatacaaatggccgag

SEQ ID NO: 252 - DNA, 89 bp, Artificial Sequence
  Synthetic
aatggaatcaacatcaaacggaaaaaaacggaattatcgaatggaatcgaagagaatcatcgaatggacccaggctggtcttgaactcc

SEQ ID NO: 253 - DNA, 82 bp, Artificial Sequence
  Synthetic
attgaatgggctagaatggaatcatctttgaacggaatcaaagggaatcatcatcgaatggaatcgaatggaaatgtcaacg

SEQ ID NO: 254 - DNA, 143 bp, Artificial Sequence
  Synthetic
aatggactcgaatggaatcaacatcaaatggaatcaagcggaattatcgaatgaaatcgaagagaatcatcgaatggactcgaaaggaatcatctaatggaatggaatggaataatccatggactcgaatgcaatcatcatcg

SEQ ID NO: 255 - DNA, 106 bp, Artificial Sequence
  Synthetic
acagacagagatttaaaacaataaacaagcagtaagcaaacacagataacaaaatgacatgatccaacaaatactcagaaggagacttagaaatgaattgagggtc

SEQ ID NO: 256 - DNA, 135 bp, Artificial Sequence
  Synthetic
aatgtaatccagcatataaacagagccaaagacaaaaaccacatgattatctcaatagatgcagaaaaagcctttgacaaaattcaacaacccttcatgctaaaaactctcaataaattaggtattgatgggacg

SEQ ID NO: 257 - DNA, 97 bp, Artificial Sequence
  Synthetic
aaacggaaaaaaacggaattattgaatggaatcgaagagaatcttcgaacggacccgaatggaatcatctaatggaatggaatggaataatccatgg

SEQ ID NO: 258 - DNA, 84 bp, Artificial Sequence
  Synthetic
gctcaaggaaataaaataggacacaaagaaatggaaaaacattccatactcatggatagaaagaatcaatatcatgaaatggcc

SEQ ID NO: 259 - DNA, 79 bp, Artificial Sequence
  Synthetic
actcgagtggaattgactgtaacaaaatggaaagtaacggattggaatcgaatggaacggaatggaatggaatggacat

SEQ ID NO: 260 - DNA, 120 bp, Artificial Sequence
  Synthetic
tacaaactttaaaaaatgatcaacagatacacagttagcaagaaagaattgagggcaaagaatatgccagacaaactcaagaggaagatgatggtagagataggtcacattggagtgtca

SEQ ID NO: 261 - DNA, 77 bp, Artificial Sequence
  Synthetic
ggaatcgaatggaatcaatatcaaacggaaaaaaacggaattatcgaatggaatcgaagagaatcatcgaatggacc

SEQ ID NO: 262 - DNA, 105 bp, Artificial Sequence
  Synthetic
aacggaatcaaacggaattatcgaatggaatcgaaaagaatcatcgaacggactcgaatggaatcatctaatgtaatggaatggaagaatccatggactcgaatg

SEQ ID NO: 263 - DNA, 84 bp, Artificial Sequence
  Synthetic
ggaaataacagagaacacaaacaaatgggaaaacattccatgttcatggataggaagaatcaatattgtgaaaatggccatact

SEQ ID NO: 264 - DNA, 105 bp, Artificial Sequence
  Synthetic
aacggaaaaaaacggaattatcgaatggaatcgaagagaatcatcgaatggaccagaatggaatcatctaatggaatggaatggaataatccatggactcgaatg

SEQ ID NO: 265 - DNA, 103 bp, Artificial Sequence
  Synthetic
caacatcaaacggaaaaaaacggaattatggaatggaatcgaagagaatcatcgaatggacccgaatggaatcatctgaaatataatagactcgaaaggaatg

SEQ ID NO: 266 - DNA, 78 bp, Artificial Sequence
  Synthetic
atggaatcgaatggaatggactggaatggaatggattcgaatggaatcgaatggaacaatatggaatggtaccaaatg

SEQ ID NO: 267 - DNA, 71 bp, Artificial Sequence
  Synthetic
gaatggaatcaacatcaaacggaaaaaaacggaattatcgaatggaatcgaagagaatcatcgaatggacc

SEQ ID NO: 268 - DNA, 105 bp, Artificial Sequence
  Synthetic
aaggaatttaagcaaatcaacaagcaaaaccaaaataatcccattaaaaagtgggtaaaggacatgaatacacacttgtcaatagaggacattcaagtggccaac

SEQ ID NO: 269 - DNA, 89 bp, Artificial Sequence
  Synthetic
aaatggactcgaatggaatcatcatagaatggaatcgaatgcaatggaatggaatcttccggaatggaatggaatggaatggaatggag

SEQ ID NO: 270 - DNA, 85 bp, Artificial Sequence
  Synthetic
gaatcancnnnnnnnggaatcgaatggaatcaacatcaaatggaatcaaatggaatcattgaacggaattgaatggaatcgtcat

SEQ ID NO: 271 - DNA, 92 bp, Artificial Sequence
  Synthetic
agcaacttcagcaaagtctcaggatacaaaatcaatgtacaaaaatcacaagcattcttatacaccaataacagacaaacagagagccaaaa

SEQ ID NO: 272 - DNA, 85 bp, Artificial Sequence
  Synthetic
tatgcaaatcaataaacataatccatcacataaacagaaacaaagacaaaatgacatgattatctcaatagatgcagaaaaggcc

SEQ ID NO: 273 - DNA, 69 bp, Artificial Sequence
  Synthetic
agtaaatcaccataaagaaggtaagagttcattcacaaaaacaacaaactgaagaatcaggccatagta

SEQ ID NO: 274 - DNA, 89 bp, Artificial Sequence
  Synthetic
agaaacagaaaacagtcaaaccaatgggcaatccatatcagatgcagtattatgaacagaagtgtaaagaatgcaccaggcacaatggc

SEQ ID NO: 275 - DNA, 98 bp, Artificial Sequence
  Synthetic
aggaaaaacaacaacaacaacaggaaaacaacctcagtatgaagacaagtacattgatttattcaacatttactgatcacttttcaggtggtaggcag

SEQ ID NO: 276 - DNA, 131 bp, Artificial Sequence
  Synthetic
gattggaacgaaatcgaatggaacggaatagaatagactcgaatgtaatggattgctatgtaattgattcgaatggaatggaatcgaatggaatgcaatccaatggaatggaatgcaatgcaatggaatgg

SEQ ID NO: 277 - DNA, 92 bp, Artificial Sequence
  Synthetic
aacatatggaaaaaaactcaacatcactgatcattagagaaatgcaaatcaaaaccacaatgagataccatctcacgccagtcagaatggcg

SEQ ID NO: 278 - DNA, 126 bp, Artificial Sequence
  Synthetic
atggaatggaataatcaacgtactcgaatgcaatcatcatcgtatagaatcgaatggaatcatcgaatggactcgaatggaataatcattgaacggagtcgaatggaatcatcatcggatggaaac

SEQ ID NO: 279 - DNA, 95 bp, Artificial Sequence
  Synthetic
aaanaantcnaatggaatcnntgncgaatggaatggaatggaatcgaanaattgaattgnnnanaatcnnangnaancnttgnatgggctcaaat

SEQ ID NO: 280 - DNA, 77 bp, Artificial Sequence
  Synthetic
agaaaagataactcgattaacaaatgaacaaacacctgaatacacaagtctcaaaagaagacataaaaatggccaac

SEQ ID NO: 281 - DNA, 102 bp, Artificial Sequence
  Synthetic
atggaatcaacatcaaacggaatcacacggaattatcgaatggaatcgaaaagaatcatcgaacggactcgaatggaatcatctaatggaatggaatggaag

SEQ ID NO: 282 - DNA, 103 bp, Artificial Sequence
  Synthetic
aatggaatcaacatcaaacggaatcaagcgaaattatcgaatggaatcgaagagaatcatcgaatggactcgaatggaatcatctaatggaatggaatgggat

SEQ ID NO: 283 - DNA, 82 bp, Artificial Sequence
  Synthetic
aaacacagtacaaatactaattcaaatcaaacttactcaaagtcataatcaaacatgccagacgggctgaggggcagcatta

SEQ ID NO: 284 - DNA, 76 bp, Artificial Sequence
  Synthetic
aaccactgctcaaggaaataagagagaacacaaacaaatgaaaaaacattccatgctcatggataggaagaatcag

SEQ ID NO: 285 - DNA, 100 bp, Artificial Sequence
  Synthetic
ggaatcgagtggaatcatcgaaagaaatcgaatggaatcattgtcgaatggaatggaatggaatcaaagaatggaatcgaagggaatcattggatgggct

SEQ ID NO: 286 - DNA, 139 bp, Artificial Sequence
  Synthetic
aaagaaagacagagaacaaacgtaattcaagatgactgtttacatatccaagaacattagatggtcaaagactttaagaaggaatacattcaaaggcaaaaagtcacttactgattttggtggagtttgccacatggac

SEQ ID NO: 287 - DNA, 90 bp, Artificial Sequence
  Synthetic
aagatagagttgaaacagtggacaattaaagagtaatttggaagaatggtgaaattacagccatgctttgaatcaggcgggttcactggc

SEQ ID NO: 288 - DNA, 77 bp, Artificial Sequence
  Synthetic
aagagtatcaacagtaaattacattagcagaagaatcaacaaacatgaaaatagaaattatggtagccaaagaacag

SEQ ID NO: 289 - DNA, 148 bp, Artificial Sequence
  Synthetic
gaaaggaatcatcattgaatgcaatcacatggaatcatcacagaatggaatcgtacggaatcatcatcgaatggaattgaatggaatcatcaattggactcgaatggaatcatcaaatggaatcgattggaagtgtcaaatggactcg

SEQ ID NO: 290 - DNA, 72 bp, Artificial Sequence
  Synthetic
cagcgcaccacagcacacacagtatacacatgacccacaatacacacaacacacaacacattcacacaccac

SEQ ID NO: 291 - DNA, 87 bp, Artificial Sequence
  Synthetic
gcaaacagaattcaacactacattagaacgatcattcatcacgacctagtaggatgtttttcctgggatgcaaggatggttcaacat

SEQ ID NO: 292 - DNA, 71 bp, Artificial Sequence
  Synthetic
caatcaaaacagcaatgagataccattttacaccaatcaaaatggctactaaaaagtcaaaagcaaatgcc

SEQ ID NO: 293 - DNA, 90 bp, Artificial Sequence
  Synthetic
agaaccatattgaagagacagagtgatatataaaactgctaactcaagcagcacaagaattaaatgaataccaagaaaatacttggccag

SEQ ID NO: 294 - DNA, 93 bp, Artificial Sequence
  Synthetic
tggaatagaatggaatcaatgttaagtggaatcgagtggaatcatcgaaagaaatcgaatggaatcattgtcgaatggtatggaatggaatca

SEQ ID NO: 295 - DNA, 124 bp, Artificial Sequence
  Synthetic
aatggaatggaatcatcgcatagaatngaatggaattatcatcgaattgaatcgaatggtatcaacatcaaacggaaaaaaacggaaatatcgaanggaatcgaagagaatcatcgaacggacc

SEQ ID NO: 296 - DNA, 95 bp, Artificial Sequence
  Synthetic
acatacgcaaatcaataaacataatccatcacataaacagaaccaaagacaaaaatcacatgattatctcaatagatgcagaaaaggccttcgac

SEQ ID NO: 297 - DNA, 83 bp, Artificial Sequence
  Synthetic
aaaaaatgttcaacatcactagtcagcagagaaatgcaaatcaaaatcacaatgagataacttctcacaccagacagcatggc

SEQ ID NO: 298 - DNA, 90 bp, Artificial Sequence
  Synthetic
gaaaaacaaaacaaaacaaacaaacaaacaatcaaaaaagtggtagcagaaaccagaaagtccatgtatatagctaattggcctggttgt

SEQ ID NO: 299 - DNA, 58 bp, Artificial Sequence
  Synthetic
aacagcaatgacaatgatcagtaacaacaagacttttaactttgaaaaaatcaggacc

SEQ ID NO: 300 - DNA, 91 bp, Artificial Sequence
  Synthetic
aagagcctgaatagctaaagtgatcataagcaaaaagaacaaagtcggaagcatcacattacctgacttcaaactatactcaaaggctatg

SEQ ID NO: 301 - DNA, 78 bp, Artificial Sequence
  Synthetic
aaaaggaaatacaagacaacaaacacagaaacacaaccatcgggcatcatgaaacctcgtgaagataatcatcagggt

SEQ ID NO: 302 - DNA, 91 bp, Artificial Sequence
  Synthetic
aagcaaagaaagaatgaagcagcaaaagaacgaaagcaggaatttattgaaaaccaaagtacactccacagtatgggagcggacccgagca

SEQ ID NO: 303 - DNA, 67 bp, Artificial Sequence
  Synthetic
gcaaatgattataagtgctgttatagaaacattcaaagaccagaaaaggaccacaatggctgaccac

SEQ ID NO: 304 - DNA, 87 bp, Artificial Sequence
  Synthetic
agagcagaaacaaatggaattgaaatgaagacaacaatcaaaagcatcaatgaaatgaaaagttgggttttggaagagagaaacaat

SEQ ID NO: 305 - DNA, 70 bp, Artificial Sequence
  Synthetic
acacaaacacacacacacacacacacacacacacacacacacacacacacacacacacacacacacatac

SEQ ID NO: 306 - DNA, 107 bp, Artificial Sequence
  Synthetic
aacaaacaaatgagatgatttcagatagtgataaacactataacataattaattcgtgccaatcagagcataacagtggtgtggtggctgtggaacagatagcagac

SEQ ID NO: 307 - DNA, 79 bp, Artificial Sequence
  Synthetic
aatggaatcgagtggaatggaaggcaatggaatagaatggaatggaatcgaaaggaacggaatggaatggaatggaatg

SEQ ID NO: 308 - DNA, 79 bp, Artificial Sequence
  Synthetic
agcagtgcaagaacaacataacatacaagtaaacaaacacatggggccaggtaataaaaagtcaggctcaagaggtcag

SEQ ID NO: 309 - DNA, 73 bp, Artificial Sequence
  Synthetic
agaaatggaatcggagagaatggaaacaaatggaatggaattgaatggaatggaattgaatggaatgggaacg

SEQ ID NO: 310 - DNA, 75 bp, Artificial Sequence
  Synthetic
gcactagtcagatcaagacagaaagtcaacgaacaaagaacagacttaaactacactctagaacaaatggaccta

SEQ ID NO: 311 - DNA, 87 bp, Artificial Sequence
  Synthetic
aagagaactgcaaaacactgctcaaagaaatcagagatgacaaaaacacatggaaaaacgtttcatgctcatggattggaagactta

SEQ ID NO: 312 - DNA, 83 bp, Artificial Sequence
  Synthetic
aatcaacacgaatagaatggaacggaatggaatggaatggaatggaatggaatggagtggaatggaacagaatggagtggaat

SEQ ID NO: 313 - DNA, 112 bp, Artificial Sequence
  Synthetic
aacatcaaacgaaatcaaacggaattatcaaattgaatcgaagagaatcatcgaattgccacgaatgcaaccatctaatggtatggaatggaataatccatggacccagatg

SEQ ID NO: 314 - DNA, 94 bp, Artificial Sequence
  Synthetic
cggaattatcatcgaatgtaatcgaatggaatcaacatcaaacggaaaaaaacggaattatcgaatggaatcgaagagaatcatcgaatggacc

SEQ ID NO: 315 - DNA, 125 bp, Artificial Sequence
  Synthetic
tggacacacacgaacacacacctacacacacgtggacacacacggacacatggacacacacgaacacatggacacacacacggggacacacacagacacacacagagacacacacggacacatgg

SEQ ID NO: 316 - DNA, 88 bp, Artificial Sequence
  Synthetic
agcaacttcagcaaagtctcaggatacaaaatcaatgtgcaaaaatcacaagcattcttatacaccaataacagacaaacagagagcc

SEQ ID NO: 317 - DNA, 96 bp, Artificial Sequence
  Synthetic
aaaatcaatatgaaaacaaacacaagcagacaaagaaaattgggcaaaaggtttgagcagacacttcaccaaagaagtacaaatggcaaatcagca

SEQ ID NO: 318 - DNA, 100 bp, Artificial Sequence
  Synthetic
atcaaacggaatcaaacggaattatcgaatggaatcgaagagaatcatcgaatggactcgaatggaatcatctaatggaatggaatggaagaatccatgg

SEQ ID NO: 319 - DNA, 101 bp, Artificial Sequence
  Synthetic
aacagatttaaacaaaccaacaagcaaaaaacgaacaactccattcaaacatggacaaaagacacgaacagacacttttcaaagaagacatacatgtggcc

SEQ ID NO: 320 - DNA, 99 bp, Artificial Sequence
  Synthetic
aaatggaatggaatgcacttgaaaggaatagactggaacaaaatgaaatcgaacggtaggaatcatacagaacagaaagaaatggaacggaatggaatg

SEQ ID NO: 321 - DNA, 68 bp, Artificial Sequence
  Synthetic
accacacacaaaatacaccacacaccacacacacaccacacactatacacacaccacacaccacacac

SEQ ID NO: 322 - DNA, 100 bp, Artificial Sequence
  Synthetic
aaagaaatagaagggagttgaacagaatcgaatggaatcgaatcaaatggaatcgaatggcatcaaatggaatcgaatggaatgtggtgaagtggattgg

SEQ ID NO: 323 - DNA, 86 bp, Artificial Sequence
  Synthetic
ggaatcatcataaaatggaatcgaatggaatcatcatcaaatggaatcaaatggaatcattgaacggaattgaatggaatcgtcat

SEQ ID NO: 324 - DNA, 77 bp, Artificial Sequence
  Synthetic
tggaatggaatggaatgaaataaacacgaatagaatggaacggaatggaacggaatggaatggaatggaatggaaag

SEQ ID NO: 325 - DNA, 87 bp, Artificial Sequence
  Synthetic
aagaattggacaaaacacacaaacaaagcaaggaaggaatgaaaggatttgttgaaaatgaaagtacactccacagtgtgggagcag

SEQ ID NO: 326 - DNA, 92 bp, Artificial Sequence
  Synthetic
taatcagcacaatcaactgtagtcacaaaacaaatagtaacgcaatgataaagaaacagagaactagttcaaataaacatgataagatgggg

SEQ ID NO: 327 - DNA, 78 bp, Artificial Sequence
  Synthetic
aagcggaattatcaaatggaatcgaagagaatggaaacaaatggaatggaattgaatggaatggaattgaatggaatg

SEQ ID NO: 328 - DNA, 97 bp, Artificial Sequence
  Synthetic
aagcaacttcagcaaagtctcaggacacaaaatcaatatgcgaaaatcacaagcattcctatacaccaataatagacaaacagagagccaaatcatg

SEQ ID NO: 329 - DNA, 62 bp, Artificial Sequence
  Synthetic
ttcacagcagcattacgcacaatagccagaaggtgggaacagacaaaatgccttttgatggg

SEQ ID NO: 330 - DNA, 88 bp, Artificial Sequence
  Synthetic
agaccctaatatcacagttaaacgaactagagaaggaagagcaaacaaattcaaaagctagcggaaagcaagaaataactaagaccag

SEQ ID NO: 331 - DNA, 86 bp, Artificial Sequence
  Synthetic
taaaagtgtgctcaacatcattgatcatcagagaaatgcaaatcaaaactacaatgagatatcatctcatcccagtcaaagtggct

SEQ ID NO: 332 - DNA, 92 bp, Artificial Sequence
  Synthetic
acttgaatcgaatggaaaggaatttaatgaacttaaatcgaatggaatataatggtatggaatggactcatggaatggaatggaaaggaatc

SEQ ID NO: 333 - DNA, 91 bp, Artificial Sequence
  Synthetic
tggaatcatcatcgaaagcaagcgaatggaatcatcaaatggaaacgaatggaatcatcgaatggactcggatggaattgttgaatggact

SEQ ID NO: 334 - DNA, 68 bp, Artificial Sequence
  Synthetic
tggaatcaacatcaaacggaaaaaaacggaattatcgaatggaatcgaagagaatcatcgaatggacc

SEQ ID NO: 335 - DNA, 86 bp, Artificial Sequence
  Synthetic
taagtgaattgaatagaatcaatctgaatgtaatgaaatggaatggaacggaatggaatggaatggaatggaatggaatggaatgg

SEQ ID NO: 336 - DNA, 98 bp, Artificial Sequence
  Synthetic
aggaaaatttaatcagcaggaatagaaacacacttgagaaatccatgtggaatgaaaagagaatggctgagcagcaacagattgtcaaaaaggaaatc

SEQ ID NO: 337 - DNA, 62 bp, Artificial Sequence
  Synthetic
aacatcaaacggaaaaaaaacggaattatcgaatggaatcgaagagaatcatcgaatggacc

SEQ ID NO: 338 - DNA, 82 bp, Artificial Sequence
  Synthetic
gaaaatgaacaatatgaacaaacaaacaaaattactacccttacgaaagtacgtgcattctagtatggtgacaaaaaggaaa

SEQ ID NO: 339 - DNA, 54 bp, Artificial Sequence
  Synthetic
agaaaacacacagacaacaaaaaacacagaacgacaatgacaaaatggccaagc

SEQ ID NO: 340 - DNA, 88 bp, Artificial Sequence
  Synthetic
agcaacttcagcaaagactcaggatacaaaatcaatgtgcaaaaatcacaagcattcttatacaccaataacagacagagagccaaat

SEQ ID NO: 341 - DNA, 97 bp, Artificial Sequence
  Synthetic
tgacatgcaagaaataaggaagtgcaaaaacaaacaaacaaacaacaacaacaacaacaacaacaacaacaaaaaacagtcccaaaaggatgggcag

SEQ ID NO: 342 - DNA, 132 bp, Artificial Sequence
  Synthetic
taattgagaataagcattccagtggaaaaaaaactaaacaatttgttgtaaaacatccttaaaagcatcagaaagttaatacagcaatgaagaattacaggaccaaattaagaatggtatggaagcctgtta

SEQ ID NO: 343 - DNA, 88 bp, Artificial Sequence
  Synthetic
tatcatcgaatggaatcgaatggaatcaacatcaaacggaaaaaaacggaattatcgaattgaatcgaagagaatcatcgaatggacc

SEQ ID NO: 344 - DNA, 74 bp, Artificial Sequence
  Synthetic
gaatggaatcaaatagaatggaatcgaaacaaatggaatggaatggaatgggagctgagattgtgtcactgcac

SEQ ID NO: 345 - DNA, 80 bp, Artificial Sequence
  Synthetic
agcaaaacaaacacaatctgtcgttcatggtactacgacatactgggagagatattcaaatgatcacacaaaacaacatg

SEQ ID NO: 346 - DNA, 93 bp, Artificial Sequence
  Synthetic
aaggattcgaatggaatgaaaaagaattgaatggaatagaacagaatggaatcaaatcgaatgaaatggagtggaatagaaaggaatggaatg

SEQ ID NO: 347 - DNA, 147 bp, Artificial Sequence
  Synthetic
aacggaatcaaacggaattatcgaatgnnntnnaagagaatcatcgaacggactcgaatggaatcatctaatggaatggaatggaagaatccatggactcgaatgcaatcatcatcgaatggaatcgaacggaatcatcgaatggcc

SEQ ID NO: 348 - DNA, 85 bp, Artificial Sequence
  Synthetic
aatcaactagatgtcaatggaatgcaatggaatagaatggaatggaattaacacgaatagaatggaatggaatggaatggaatgg

SEQ ID NO: 349 - DNA, 76 bp, Artificial Sequence
  Synthetic
tgtaacactgcaaaccataaaaaccgtagaagaaaacctagacaatactattcaggacataggcatgggcaaagac

SEQ ID NO: 350 - DNA, 88 bp, Artificial Sequence
  Synthetic
aatggactcgaatggaataatcattgaacggaatcgaatggaatcatcatcggatggaaatgaatggaatcatcatcgcatggaatcg

SEQ ID NO: 351 - DNA, 76 bp, Artificial Sequence
  Synthetic
gaatggaatgatacggaatagaatggaatggaacgaaatggaattgaaaggaaaggaatggaatggaatggaatgg

SEQ ID NO: 352 - DNA, 79 bp, Artificial Sequence
  Synthetic
aaaaatgaccagagcaatagaatgcattgaccagataaagaccttcacgtatgttgaactaaaatgtgtggtgcaggtg

SEQ ID NO: 353 - DNA, 84 bp, Artificial Sequence
  Synthetic
aatcatcatcgaatggaatcgaatggtatcattgantgnaatcgaatggaatcatcatcanatggaaatgaatggaatcgtcat

SEQ ID NO: 354 - DNA, 86 bp, Artificial Sequence
  Synthetic
acaaaatcaaactaacctcgataagaatgcaagtgaatcaaaatgagtttcaaggggttgtggctagtacacgctttctacagctg

SEQ ID NO: 355 - DNA, 65 bp, Artificial Sequence
  Synthetic
gaatcaaatcaatggaatcaaatcaaatggaatggaatggaattgtatggaatggaatggcatgg

SEQ ID NO: 356 - DNA, 125 bp, Artificial Sequence
  Synthetic
taatgcagtccaatagaatggaatcgaatggcatggaatataaagaaatggaatcgaagagaatgggaacaaatggaatggaattgagtggaatggaattgaatggaatgggaacgaatggagtg

SEQ ID NO: 357 - DNA, 91 bp, Artificial Sequence
  Synthetic
tgaatagacacacagaccaatggaacagaatagagaacacagaataaatctgcacacttatagccagctgatttttgacaaatttgccaag

SEQ ID NO: 358 - DNA, 61 bp, Artificial Sequence
  Synthetic
aacatcnnacggaaaaaaacggaattatcgaatggaatcgaagagaatcatcgaatggacc

SEQ ID NO: 359 - DNA, 99 bp, Artificial Sequence
  Synthetic
gccaacaatcatatgagaaaaagctcaacatcactgatcatttcaggaatgcaaatcaaaaccacaatgagatactatcacacatcaatcagaatggct

SEQ ID NO: 360 - DNA, 76 bp, Artificial Sequence
  Synthetic
gaatcgaatggaatcaacatcaaacggaaaaaaacggaattatcgaatggaatcgaagagaatcatcgaatggacc

SEQ ID NO: 361 - DNA, 99 bp, Artificial Sequence
  Synthetic
aatcaaatggaatgaaatcgaatggaattgaatcgaatggaatgcaatagaatgtcttcaaatggaatcgaatggaaattggtgaagtggacgggagtg

SEQ ID NO: 362 - DNA, 122 bp, Artificial Sequence
  Synthetic
taacagtaccaaaaaacagtcataatcttcaagagcttaaatttagcatgaaaggaagacattcatcaaagaatcacacaaaggaatgtaaaattaaatggagattagtgccaggaaagagc

SEQ ID NO: 363 - DNA, 71 bp, Artificial Sequence
  Synthetic
taatggaatcaacatcaaacggaaaaaaacggaattatcgaatgcaatcgaagagaatcatcgaatggacc

SEQ ID NO: 364 - DNA, 88 bp, Artificial Sequence
  Synthetic
agcaacttcagcaaagtctcagcatacaaaatcaatgtgcaaaaatcacacgcattcctatacaccaataacagacaaacagagagcc

SEQ ID NO: 365 - DNA, 70 bp, Artificial Sequence
  Synthetic
gaatcaaatggaatggactgtaatggaatggattcgaatggaatcgaatggagtggactcaaatggaatg

SEQ ID NO: 366 - DNA, 93 bp, Artificial Sequence
  Synthetic
aacaagtggacgaaggatatgaacagacacttctcaagacatttatgcagccaacagacacacgaaaaaatgctcatcatcactggccatcag

SEQ ID NO: 367 - DNA, 85 bp, Artificial Sequence
  Synthetic
aaacacacaaagcaacaaaagaacgaagcaacaaaagcatagatttattgaaatgaaagtacattctacagagtgggggcaggct

SEQ ID NO: 368 - DNA, 90 bp, Artificial Sequence
  Synthetic
atacaactaaagcaaatataagcaactaaagcaacagtacaactaaagcaaaacagaacaagactgccagggcctagaaaagccaagaac

SEQ ID NO: 369 - DNA, 68 bp, Artificial Sequence
  Synthetic
gcaatcgaatggaatggaatcgaacggaatggaataaaatggaagaaaactggcaagaaatggaatcg

SEQ ID NO: 370 - DNA, 72 bp, Artificial Sequence
  Synthetic
agcagccaacaagcatatgaaataatgctccacaacactcatcatcagagaaatgcaaatcaaaaccaaaat

SEQ ID NO: 371 - DNA, 105 bp, Artificial Sequence
  Synthetic
tggaaccgaacaaagtcatcaccgaatggaattgaaatgaatcataatcgaatggaatcaaatggcatcttcgaattgactcgaatgcaatcatccactgggctt

SEQ ID NO: 372 - DNA, 84 bp, Artificial Sequence
  Synthetic
aacggaatcacgcggaattatcgaatggaatcgaagagaatcatcgaatggactcgaatggaatcatctaatggaatggaatgg

SEQ ID NO: 373 - DNA, 90 bp, Artificial Sequence; identical to SEQ ID NO: 293
  Synthetic

SEQ ID NO: 374 - DNA, 68 bp, Artificial Sequence
  Synthetic
aaaacaaacaacaacgacaaatcatgagaccagagttaagaaacaatgagaccaggctgggtgtggtg

SEQ ID NO: 375 - DNA, 80 bp, Artificial Sequence
  Synthetic
aatcgaaaggaatgcaatattattgaacagaatcgaaaagaatggaatcaaatggaatggaacagagtggaatggactgc

SEQ ID NO: 376 - DNA, 79 bp, Artificial Sequence
  Synthetic
aaggaatcgaatggaagtgaatgaaattgaatcaacaggaatggaagggaatagaatagactgtaatggaatggactcg

SEQ ID NO: 377 - DNA, 85 bp, Artificial Sequence
  Synthetic
aatggactcgaatgaaatcatcatcaaacggaatcgaatggaatcattgaatggaatggaatggaatcatcatggaatggaaacg

SEQ ID NO: 378 - DNA, 78 bp, Artificial Sequence
  Synthetic
ttgaccagaacacattacacaatgctaatcaactgcaaaggagaatatgaacagagaggaggacatggatattttgtg

SEQ ID NO: 379 - DNA, 65 bp, Artificial Sequence
  Synthetic
aacccgagtgcaatagaatggaatcgaatggaatggaatggaatggaatggaatggaatggagtc

SEQ ID NO: 380 - DNA, 92 bp, Artificial Sequence
  Synthetic
aagagtattgaagttgacatatctagactgatcaagaacaaagacaaaaggtacagattatcaagaaaatgagcgggcaaagcaagatggcc

SEQ ID NO: 381 - DNA, 80 bp, Artificial Sequence
  Synthetic
gaatggaattgaaaggaatggaatgcaatggaatggaatgggatggaatggaatgcaatggaatcaactcgattgcaatg

SEQ ID NO: 382 - DNA, 101 bp, Artificial Sequence
  Synthetic
gaaaaaaacggaattatcnaattgaatcnaatanaatcatcnnnnngaccanantggaatcatctaatgnaatgnaatggaataatccatggactcnaatg

SEQ ID NO: 383 - DNA, 101 bp, Artificial Sequence
  Synthetic
gaaaaaaacggaattatcgaattgaatcgaatagaatcatcgaacggaccagaatggaatcatctaatggaatggaatggaataatccatggactcgaatg

SEQ ID NO: 384 - DNA, 91 bp, Artificial Sequence
  Synthetic
aaccactgcttaaggaaataagagagaacacaaacaaatggaaaaacgttccatgctcatggataggagaatcaatatcgtgaaaatggcc

SEQ ID NO: 385 - DNA, 95 bp, Artificial Sequence
  Synthetic
tatcgaatggaatggaaaggagtggagtagactcgaatagaatggactggaatgaaatagattcgaatggaatggaatggaatgaagtggactcg

SEQ ID NO: 386 - DNA, 84 bp, Artificial Sequence
  Synthetic
gtatcaacatcaaacggaaaaaaacggaattatcgaatggaatcatctaatggaatggaatggaataatccatggactcgaatg

SEQ ID NO: 387 - DNA, 87 bp, Artificial Sequence
  Synthetic
taaatggagacatcattgaatacaattgaatggaatcatcacatggaatcgaatggaatcatcgtaaatgcaatcaagtggaatcat

SEQ ID NO: 388 - DNA, 83 bp, Artificial Sequence
  Synthetic
gaatggaattgaaaggtatcaacaccaaacggaaaaaaaaacggaattatcgaatggaatcgaagagaatcatcgaacggacc

SEQ ID NO: 389 - DNA, 73 bp, Artificial Sequence
  Synthetic
agcaatttcagcaaagtctcaggatacaaaatcaatgtacaaattcacaagcattcttatggaccaacaacag

SEQ ID NO: 390 - DNA, 88 bp, Artificial Sequence
  Synthetic
aaccaaattagacaaattggaaatcattacacataacaaaagtaataaactgtcagcctcagtagtattcattgtacataaactggcc

SEQ ID NO: 391 - DNA, 109 bp, Artificial Sequence
  Synthetic
tattttaccagattattcaagcaatatatagacagcttaaagcatacaagaagacatgtatagatttacatgcaaacactgcaccactttacataagggacttgagcac

SEQ ID NO: 392 - DNA, 68 bp, Artificial Sequence
  Synthetic
ggaatcgaatggcatcaacatcaaacggaaaaaaacggaattatcgaatggaatcgaatggaatcatc

SEQ ID NO: 393 - DNA, 90 bp, Artificial Sequence
  Synthetic
aaacaaaacacagaaatgcaaagacaaaacataaaacgcagccataaaggacatattttagataactggggaaatttgtatgggctgtgt

SEQ ID NO: 394 - DNA, 84 bp, Artificial Sequence
  Synthetic
aggaaaagaaagaaatagaaaatgcgaaatggtaagaaaaaacagcataataaacatttgtatggtgttgatggacaatgcatt

SEQ ID NO: 395 - DNA, 103 bp, Artificial Sequence
  Synthetic
aatggaatcaacatcaaacggaatcaaacggaattatcgaatggaatcgaagagaatcatcgaacggactcgaatggaatcatctaatggaatggaatggaag

SEQ ID NO: 396 - DNA, 75 bp, Artificial Sequence
  Synthetic
aatcgaatggaatcagcatcaaacggaaaaaaacggaattatcgaatggaatcgaagagaatcatcgaatggacc

SEQ ID NO: 397 - DNA, 75 bp, Artificial Sequence
  Synthetic
aaagaaatggaatcgaagagaatggaaacaaatggaatggaattgaatggaatggaattgaatggaatgggaacg

SEQ ID NO: 398 - DNA, 90 bp, Artificial Sequence
  Synthetic
agaaagaatcaagaggaaatgcaagaaatccaaaacactgtaacagatatgatgaataatgaggtatgcactcatcagcagactcgacat

SEQ ID NO: 399 - DNA, 94 bp, Artificial Sequence
  Synthetic
aaacggaattatnnantggannnnaagnnaatcatcgaacggannnnannggaatcatntnnnngaanggaatggaacaatccatggtntnnnn

SEQ ID NO: 400 - DNA, 88 bp, Artificial Sequence
  Synthetic
agcaacttcagcaaagtttcaggatacaaaatcaatgtgcaaaaatcacaagcattcttatacaccaacaacagacaaacagagagcc

SEQ ID NO: 401 - DNA, 87 bp, Artificial Sequence
  Synthetic
agacagtcagacaatcacaaagaaacaagaatgaaaatgaatgaacaaaaccttcaagaaatatgggattatgaagaggccaaatgt

SEQ ID NO: 402 - DNA, 99 bp, Artificial Sequence
  Synthetic
atcataacgacangancaaattcacacacaacaatnnnnacnnnaaanncaaatgggttaaatnntncaattaaaggatgcagacgggcaaattggata

SEQ ID NO: 403 - DNA, 99 bp, Artificial Sequence
  Synthetic
atcataangacaagancaaattcacacacaacaatnnnnacnnnaaanncaaatgggttnaatgntncaattaaaggatgcagacggncaaattggata

SEQ ID NO: 404 - DNA, 95 bp, Artificial Sequence
  Synthetic
gaatggaatcgaatggattgatatcaactggaatggaatggaagggaatggaatggaatggaattgaaccaaatgtnnnngncttgaatggaatg

SEQ ID NO: 405 - DNA, 66 bp, Artificial Sequence
  Synthetic
gaatcaacatcaaacggaaaaaaacggaattatcgaatggaatcgaagagaatcatcgaatggacc

SEQ ID NO: 406 - DNA, 135 bp, Artificial Sequence
  Synthetic
atggaatcaacatcaaacggaatcaaacggaattatcgaatggaatcaaagagaatcatcgaacggactcgaatggaatcatctaatggaatggaatggaagaatccatggactcgaatgcaatcatcatcgaat

SEQ ID NO: 407 - DNA, 52 bp, Artificial Sequence
  Synthetic
ggaatggaatggaatggagcngaatngaanggannnnantcaaatggaatgc

SEQ ID NO: 408 - DNA, 90 bp, Artificial Sequence
  Synthetic
aacatacgcaaatcaataaatgtaatccagcatataaacagaaccaaagacaaaaaccacatgattatctcaatagatgcagaaaaggcc

SEQ ID NO: 409 - DNA, 124 bp, Artificial Sequence
  Synthetic
aaacgattggacaggaatggaatcaccatcgaatggaaacgaatggaatcttcgaatggaattgaatgaaattattgaacggaatcaaatagaatcatcattgaacagaatcaaattggatcat

SEQ ID NO: 410 - DNA, 79 bp, Artificial Sequence
  Synthetic
aaaagatgcaaaagtagcaaatgcaatgttaaaacaagcaaagaaagaatcaggtggaccacatagtgcagtgcttctc

SEQ ID NO: 411 - DNA, 85 bp, Artificial Sequence
  Synthetic
aacaataaacaaactccaactagacacaatagtcaaattgctgaaaatgaaatataaaggaacaatctcgatggtagcccaagga

SEQ ID NO: 412 - DNA, 90 bp, Artificial Sequence
  Synthetic
agtcaataacaagaagacaaacaacccaattacaaaatgggatatgaatttaatagatgttactccaaggaagatacacaaatggccaac

SEQ ID NO: 413 - DNA, 150 bp, Artificial Sequence
  Synthetic
aaaacacctaggaatacagataacaagggacattaactacctcttaaagagaactacaaaccactgctcaaggaaatgagagaggacacaaacacatggaaaaacattccatcctcatggataggaagaatcaatattgtgaaaatggcc

SEQ ID NO: 414 - DNA, 93 bp, Artificial Sequence
  Synthetic
gatatataaacaagaaaacaactaatcacaactcaatatcaaagtgcaatgatggtgcaaaatgcaagtatggtggggacagagaaaggatgc

SEQ ID NO: 415 - DNA, 66 bp, Artificial Sequence
  Synthetic
acacatatcaaacaaacaaaagcaattgactatctagaaatgtctgggaaatggcaagatattaca

SEQ ID NO: 416 - DNA, 86 bp, Artificial Sequence
  Synthetic
ggaatcatcatataatggaatcgaatggaatcaacatcaaatggaatcaaatggaatcattgaacggaattgaatggaatcgtcat

SEQ ID NO: 417 - DNA, 89 bp, Artificial Sequence
  Synthetic
cccaacttcaaattatactacaaggctacagtaatcaaaaaagcatagtactattacaaaaacagacacacaggccaatggaatacaat

SEQ ID NO: 418 - DNA, 103 bp, Artificial Sequence
  Synthetic
aaacgcagaaacaaatcaacgaaagaacgaagcaatgaaagacaaagcaacaaaagaatggagtaagaaagcacactccacaaagtggaagcaggctgggaca

SEQ ID NO: 419 - DNA, 95 bp, Artificial Sequence
  Synthetic
aactaacacaagaacagaaaaccaaacatcacatgttctcactcataagcgggagctgaacaatgagaacacacggacacagggagaggaacatg

SEQ ID NO: 420 - DNA, 89 bp, Artificial Sequence
  Synthetic
gccacaattttgaaacaaccataataatgagaatacacaagacaactccaataatgtgggaagacaaactttgcaattcacatcatggc

SEQ ID NO: 421 - DNA, 121 bp, Artificial Sequence
  Synthetic
aatggaatcaacatcaaacggaatcaaatggaattatcgaatggaatcgaagagaatcatcgaattgtcacgaatggaatcatctaatggaatggaatggaataatccatggcccctatgc

SEQ ID NO: 422 - DNA, 56 bp, Artificial Sequence
  Synthetic
taaacagaaccaaagacaaaaatcacatgattatctcaatagatgcagaaaaggcc

SEQ ID NO: 423 - DNA, 83 bp, Artificial Sequence
  Synthetic
atcaacagacaacagaaacaaatccacaaagcacttagttattagaactgtcatacagactgtacaacaaccacatttaccat

SEQ ID NO: 424 - DNA, 85 bp, Artificial Sequence
  Synthetic
aatggactcgaatgaaatcatcatcaaacagaatcgaatggaatcatctaatggaatggaatggcataatccatggactcgaatg

SEQ ID NO: 425 - DNA, 91 bp, Artificial Sequence
  Synthetic
taaaatgaaacaaatatacaacacgaaggttatcaccagaaatatgccaaaacttaaatatgagaataagacagtctcaggggccacagag

SEQ ID NO: 426 - DNA, 59 bp, Artificial Sequence
  Synthetic
aaaatacagcgttatgaaaagaatgaacacacacacacacacacacacacagaaaatgt

SEQ ID NO: 427 - DNA, 77 bp, Artificial Sequence
  Synthetic
tactctcagaagggaagcagatattcagcataaatcatattgtttgtacaaagagtctgggcatggtgaatgacact

SEQ ID NO: 428 - DNA, 138 bp, Artificial Sequence
  Synthetic
caaacaaataggtaccaaacaaataacaacataaacctgacaacacacttatttacaagagacatcccttatatgaaagggtacagaaaagtcgatggtaagatgatggggaaaggtataccaaccactagcagaagg

SEQ ID NO: 429 - DNA, 93 bp, Artificial Sequence
  Synthetic
tggaatcgaatggaatcaatatcaaacggaaaaaaacggaattatcgaatggaatcgaaaagaatcatcgaatgggcccgaatggaatcatct

SEQ ID NO: 430 - DNA, 85 bp, Artificial Sequence
  Synthetic
acaaatggaatcaacaacgaatggaatcgaatggaaacgccatcgaaaggaaacgaatggaattatcatgaaattgaaatggatg

SEQ ID NO: 431 - DNA, 91 bp, Artificial Sequence
  Synthetic
gacaagagttcagaaaggaagactacacagaaatacgcattttaaagtcactgacatggagatgacacttaaaaccatgaacatggatggg

SEQ ID NO: 432 - DNA, 94 bp, Artificial Sequence
  Synthetic
aaaataaacgcaaattaaaatcacaagataccaacacattcccacggctaagtacgaagaacaagggcgaatggtcagaattaagctcaaacct

SEQ ID NO: 433 - DNA, 89 bp, Artificial Sequence
  Synthetic
taaactgacacaaacacagacacacagatacacacatacatacagaaatacacattcacacacagacctggtctttggagccagagatg

SEQ ID NO: 434 - DNA, 81 bp, Artificial Sequence
  Synthetic
gatcaataaatgtaattcatcatataaacagagaactaaagacaaaaacacatgattatcgcaatacatgcagaaaaggcc

SEQ ID NO: 435 - DNA, 98 bp, Artificial Sequence
  Synthetic
aggacatgaatagacaattctcaaaagaagatacacaagtggcaaacaaacacatgaaaaaagactcaacattagtaatgaccatggaaatgcaaatc

SEQ ID NO: 436 - DNA, 60 bp, Artificial Sequence
  Synthetic
acatcaaacggaaaaaaacggaattatcgaatggaatcgaagagaatcatcgaatggacc

SEQ ID NO: 437 - DNA, 70 bp, Artificial Sequence
  Synthetic
aatggactcgaatagaattgactggaatggaatggactcgaatggaatggaatggaatggaagggactcg

SEQ ID NO: 438 - DNA, 138 bp, Artificial Sequence
  Synthetic
aagaaagacagagaacaaacgtaattcaagatgactgattacatatccaagaacattagatggtcaaagactttaagaaggaatacattcaaaggcaaaacgtcacttactgattttggtggagtttgccacatggac

SEQ ID NO: 439 - DNA, 75 bp, Artificial Sequence
  Synthetic
aacataatccatcaaataaacagaaccaaagacaaaaaccacatgattatctcaatagatgcagaaaaggccttc

SEQ ID NO: 440 - DNA, 83 bp, Artificial Sequence
  Synthetic
gaatggaatcgaatggaatgaacatcaaacggaaaaaaacggaattatcgaatggaatcaaagagaatcatcgaatggacccg

SEQ ID NO: 441 - DNA, 96 bp, Artificial Sequence
  Synthetic
atggactcgaatgtaataatcattgaacggaatcgaatggaatcatcatcggatggaaacgaatggaatcatcatcgaatggaatcgaatgggatc

SEQ ID NO: 442 - DNA, 113 bp, Artificial Sequence
  Synthetic
gaatggaatcaacatcaaacggaatcaaacggaattatcgaatggaatcgaagagaatcatcgaatggccacgaatggaatcatctaatggaatggaatggaataatccatgg

SEQ ID NO: 443 - DNA, 66 bp, Artificial Sequence
  Synthetic
gaaatggaatggaaaggaataaaatcaagtgaaattggatggaatggattggaatggattggaatg

SEQ ID NO: 444 - DNA, 98 bp, Artificial Sequence
  Synthetic
aaacggaaaaaaaacggaattatcgaatggaatcgaagagaatcatcgaacgaaccagaatggaatcatctaatggaatggaatggaataatccatgg

SEQ ID NO: 445 - DNA, 78 bp, Artificial Sequence
  Synthetic
attaacccgaatagaatggaatggaatggaatggaacggaacggaatggaatggaatggaatggaatggaatggatcg

SEQ ID NO: 446 - DNA, 82 bp, Artificial Sequence
  Synthetic
gcaaaacacaaacaacgccataaaaaactgggcaaaggatatgaacagacatttttcaaaacaaaacatacttatggccaac

SEQ ID NO: 447 - DNA, 61 bp, Artificial Sequence
  Synthetic
aacatcaaacggaaaaaaacggaattatcgtatggaatcgaagagaatcatcgaatggacc

SEQ ID NO: 448 - DNA, 84 bp, Artificial Sequence
  Synthetic
aaatcaataaatgtaattcagcatataaacagaaccaaagacaaaaaccacatgattatctcaatagatgcagaaaaggccttt

SEQ ID NO: 449 - DNA, 71 bp, Artificial Sequence
  Synthetic
agaatcaaatggaattgaatcgaatggaatcgaatggattggaaaggaatagaatggaatggaatggaatg

SEQ ID NO: 450 - DNA, 86 bp, Artificial Sequence
  Synthetic
gaatagaattgaatcatcattgaatggaatcgagtagaatcattgaaatcgaatggaatcatcatcgaatggaattgggtggaatc

SEQ ID NO: 451 - DNA, 148 bp, Artificial Sequence
  Synthetic
caccgaatagaatcgaatggaacaatcatcgaatggactcaaatggaattatcctcaaatggaatcgaatggaattatcgaatgcaatcgaatagaatcatcgaatagactcgaatggaatcatcgaatggaatggaatggaacagtc

SEQ ID NO: 452 - DNA, 85 bp, Artificial Sequence
  Synthetic
aaatcatcatcgaatggaatcgaatggtatcattgaatggaatcgaatggaatcatcatcagatggaaatgaatggaatcgtcat

SEQ ID NO: 453 - DNA, 95 bp, Artificial Sequence
  Synthetic
gaatggaatcgaaaggaatagaatggaatggatcgttatggaaagacatcgaatggaatggaattgactcgaatggaatggactggaatggaacg

SEQ ID NO: 454 - DNA, 86 bp, Artificial Sequence; identical to SEQ ID NO: 450
  Synthetic

SEQ ID NO: 455 - DNA, 110 bp, Artificial Sequence
  Synthetic
gaaaggaatagaatggaatggatcgttatggaaagacatcgaatgggatggaattgactcgaatggattggactggaatggaacggactcgaatggaatggactggaatg

SEQ ID NO: 456 - DNA, 88 bp, Artificial Sequence
  Synthetic
tggatttcagatatttaacacaaaatagtcaaagcagataaatactagcaacttatttttaatgggtaacatcatatgttcgtgcctt

SEQ ID NO: 457 - DNA, 88 bp, Artificial Sequence
  Synthetic
acagcagaaaacgaacatcagaaaatcactctacatgatgcttaaatacagagggcaagcaacccaagagaaaacaccacttcctaat

SEQ ID NO: 458 - DNA, 90 bp, Artificial Sequence
  Synthetic
aacatacacaaatcaataaacgtaatccagcttataaacagaaccaaagacaaaaaccacatgattatctcaatagatgcggaaaaggcc

SEQ ID NO: 459 - DNA, 93 bp, Artificial Sequence
  Synthetic
acatcaaacggaatcaaacggaattatcgaatggaatcgaaaagaatcatcgaacggactcgaatggaatcatctaatggaatggaatggaag

SEQ ID NO: 460 - DNA, 74 bp, Artificial Sequence
  Synthetic
atcgaatggaatcaacatcaaacggaaaaaaacggaattatcaaatggaatcgaagagaatcatcgaatggacc

SEQ ID NO: 461 - DNA, 86 bp, Artificial Sequence
  Synthetic
gaataatcattgaacggaatcgaatggaaacatcatcgaatggaaacgaatggaatcatcatcgaatggaaatgaaaggagtcatc

SEQ ID NO: 462 - DNA, 110 bp, Artificial Sequence
  Synthetic
catcaaacggaatcaaacggaattatcgaatggaatcgaaaagaatcatcgaacggactcgaatggaatcatctaatggaatggaatggaagaatccatggactcgaatg

SEQ ID NO: 463 - DNA, 92 bp, Artificial Sequence
  Synthetic
tccagtcgatcatcatatagtcagcacttatcatacaccaagccgtgtgcaaggaaagggaatacaaccatgaacatgatagatggatggtt

SEQ ID NO: 464 - DNA, 96 bp, Artificial Sequence
  Synthetic
acaaaccactgctcaaggaaataaggacacaaacaaatggaacaacattccgtgctcatggataggaagaatcaatatcgtgaaaatggccatact

SEQ ID NO: 465 - DNA, 87 bp, Artificial Sequence
  Synthetic
acaaaattgatagaccactagcaagactaataaagaagaaaagagagaagaatcattaccattcaggacataggcatgggcaaggac

SEQ ID NO: 466 - DNA, 97 bp, Artificial Sequence
  Synthetic
aaacggaatcaaacggaattatcgaatggaatcgaagagaatcatcgaatggactcgaatggaatcatctaatggaatggaatggaagaatccatgg

SEQ ID NO: 467 - DNA, 87 bp, Artificial Sequence
  Synthetic
atacacaaatcaataaatgtaatccagcatataaacagaaccaaagacaaaaaccatatgattatctcaatggatgcagaaaaggcc

SEQ ID NO: 468 - DNA, 61 bp, Artificial Sequence
  Synthetic
aatngaatagaatcatcgaatggactcgaatggaatcatcgannntantgatggaacagtc

SEQ ID NO: 469 - DNA, 124 bp, Artificial Sequence
  Synthetic
tggaatggaatcatcgcatagaatcgaatggaattaccatcgaatgggatcgaatggtatcaacatcaaacgcaaaaaaacggaattatcgaatggaatcgaagagaatcttcgaacggacccg

SEQ ID NO: 470 - DNA, 75 bp, Artificial Sequence
  Synthetic
gaattgaattgaatggaatggaatgcaatggaatctaatgaaacggaaaggaaaggaatggaatggaatggaatg

SEQ ID NO: 471 - DNA, 90 bp, Artificial Sequence
  Synthetic
aacagaatggaatcaaatcgaatgaaatggaatggaatagaaaggaatggaatgaaatggaatggaaaggattcgaatggaatgcaatcg

SEQ ID NO: 472 - DNA, 63 bp, Artificial Sequence
  Synthetic
atggaatggaatggaatggaattaaatggaatggaaaggaatggaatcgaatggaaaggaatc

SEQ ID NO: 473 - DNA, 102 bp, Artificial Sequence
  Synthetic
gtcgaaatgaatagaatgcaatcatcatcaaatggaatccaatggaatcatcatcaaatagaatcgaatggaatcatcaaatggaatcgaatggagtcattg

SEQ ID NO: 474 - DNA, 77 bp, Artificial Sequence
  Synthetic
tggaattatcgaaagcaaacgaatagaatcatcgaatggactcgaatggaatcatcgaatggaatggaatggaacag

SEQ ID NO: 475 - DNA, 69 bp, Artificial Sequence
  Synthetic
aaaggaatggaatgcaatggaatgcaatggaatgcacaggaatggaatggaatggaatggaaaggaatg

SEQ ID NO: 476 - DNA, 75 bp, Artificial Sequence
  Synthetic
aatctaatggaatcaacatcnaacggaaaaaaacggaattatcgaatggaatcnaagagaatcatcnaatggacc

SEQ ID NO: 477 - DNA, 100 bp, Artificial Sequence
  Synthetic
tacacaacaaaagaaatactcaacacagtaaacagacaaccttcagaacaggagaaaatatttgcaaatacatctaacaaagggctaatatccagaatct

SEQ ID NO: 478 - DNA, 70 bp, Artificial Sequence
  Synthetic
ngcaatcntagtntcagataaaacagacattaaaccaacaaagatcaaaagagacaaagaaggccantac

SEQ ID NO: 479 - DNA, 76 bp, Artificial Sequence
  Synthetic
gaatcgaatggaatcaacatcaaacggaaaaaaacggaattatcgaatggaatcnaaaagaatcatcnaatggacc

SEQ ID NO: 480 - DNA, 92 bp, Artificial Sequence
  Synthetic
acagttaacaaaaaccgaacaatctaattacgaaatgaacaaaagatatgaacagacatttcacccgagagtatacaggggccaggcatggt

SEQ ID NO: 481 - DNA, 85 bp, Artificial Sequence
  Synthetic
aatggaatcgaatggaatgcaatccaatggaatggaatgcaatgcaatggaatggaatcgaacggaatgcagtggaagggaatgg

SEQ ID NO: 482 - DNA, 102 bp, Artificial Sequence
  Synthetic
gaacacagaaaaatttcaaaggaataatcaacagggattgataactaactggatttagagagccaaggcaaagagaatcaaagcacagggcctgagtcggag

SEQ ID NO: 483 - DNA, 84 bp, Artificial Sequence
  Synthetic
tataccacacaaatgcaaaagattattagcaacaattatcaacagcaatatgtcaacaagttgacaaacctagaggacatggat

SEQ ID NO: 484 - DNA, 86 bp, Artificial Sequence
  Synthetic
caccatgagtcattaggtaaatgcaaatcaaaaccacaatgaaatacttcacacccatgaagatggctataataaaaaaacagaca

SEQ ID NO: 485 - DNA, 81 bp, Artificial Sequence
  Synthetic
agttgaatagaaccaatccgaatgaaatggaatggaatggaacggaatggaattgaatggaatggaatggaatgcaatgga

SEQ ID NO: 486 - DNA, 101 bp, Artificial Sequence
  Synthetic
aagtaataagactgaattagtaatacaaagtgtctcaacaaagaaaattgcgggactgttcatgctcatggacaggaagaatcaatatcatgaaaatggcc

SEQ ID NO: 487 - DNA, 90 bp, Artificial Sequence
  Synthetic
aactcgattgcaatggaatgtaatgtaatggaatggaatggaattaacgcgaatagaatggaatggaatgtaatggaacggaatggaatg

SEQ ID NO: 488 - DNA, 82 bp, Artificial Sequence
  Synthetic
aagcggaatagaattgaatcatcattgaatggaatcgagtagaatcattgaaatcgaatggaatcatagaatggaatccaat

SEQ ID NO: 489 - DNA, 91 bp, Artificial Sequence
  Synthetic
aaaggaaaactacaaaacactgctgaaagaaatcattgacaacacaaacaaatggaaacacatcccaagatcatgggtgggtggaatcaat

SEQ ID NO: 490 - DNA, 94 bp, Artificial Sequence
  Synthetic
aatggaatcnaaaggaatagaatggaatggatcgttatggaaagatatcgaatggaatggaattgactcgaatggaatggactggaatggaacg

SEQ ID NO: 491 - DNA, 91 bp, Artificial Sequence
  Synthetic
taacggaataatcatcgaacagaatcaaatggaatcatcattgaatggaattgaatggaatcttcgaatagacatgaatggaccatcatcg

SEQ ID NO: 492 - DNA, 105 bp, Artificial Sequence
  Synthetic
aaagaccgaaacaacaacagaaacagaaacaaacaacaataagaaaaaatgttaagcaaaacaaatgattgcacaacttacatgattactgagtgttctaatggt

SEQ ID NO: 493 - DNA, 92 bp, Artificial Sequence
  Synthetic
aagatttaaacataagacctaaaacgacaaaaatcctaggagaaaacctaagcaataccattcaggacataggcatgggcaaagacttcatg

SEQ ID NO: 494 - DNA, 99 bp, Artificial Sequence
  Synthetic
agaaacagccagaaaacaattattacctacagcattaaaactattcaaatgacagcatatttttcagcagaaatcatgaaggccagaaggacgtgtcat

SEQ ID NO: 495 - DNA, 92 bp, Artificial Sequence
  Synthetic
atgtacacaaatcaataaatgcagtccagcatataaacagaaccaaacacaaaaaccacatgattatctcaatagatgcagaaaaggccttt

SEQ ID NO: 496 - DNA, 88 bp, Artificial Sequence
  Synthetic
agcaacttcagcaaagtctcaggacacaaaatcaatgtgcaaaaatcacaagcattcttatacaccaataacagacaaacagagagcc

SEQ ID NO: 497 - DNA, 72 bp, Artificial Sequence
  Synthetic
ttgaatcgaatggaatcgaatggattggaaaggaatagaatggaatggaatggaattgactcaaatggaatg

SEQ ID NO: 498 - DNA, 87 bp, Artificial Sequence
  Synthetic
aacggaatcaaacggaattatcgaatggaatcgaatagaatcatcgaacggactcgaatggaatcatctaatggaatggaatggaag

SEQ ID NO: 499 - DNA, 76 bp, Artificial Sequence
  Synthetic
aacatcactgatcattagaaacacacaaatcaaaaccacaataagataccatctaacaccagtcacaatggctatt

SEQ ID NO: 500 - DNA, 94 bp, Artificial Sequence
  Synthetic
taagcaatttcagcagtctcaggatacaaaatcaatgtgcaaaaatcacaagcattcttatacaccaacaacagacaaacagagagccaaatcg

SEQ ID NO: 501 - DNA, 85 bp, Artificial Sequence
  Synthetic
agaaaaaaacaaacagcccattaaaaggtagacaaaggacatgaacacttttcaaaagaagacatacatgtggccaaacagcatg

SEQ ID NO: 502 - DNA, 84 bp, Artificial Sequence
  Synthetic
attggaatggaacggaacagaacggaatggaatggaatagaatggaatggaatggaatggtatggaatggaatggaatggtacg

SEQ ID NO: 503 - DNA, 80 bp, Artificial Sequence
  Synthetic
agagcatccacaaggcccaattcaaagaatctgaaataatgtattgttactgcaacagttgtgagtaccagtggcatcag

SEQ ID NO: 504 - DNA, 87 bp, Artificial Sequence
  Synthetic
aatccacaaagacaacagaagaaaagacaacagtagacaaggatgtcaaccacattttggaagagacaagtaatcaaacacatggca

SEQ ID NO: 505 - DNA, 106 bp, Artificial Sequence
  Synthetic
aaacagaaccacagatatctgtaaaggattacactatagtattcaacagagtatggaacagagtatagtattcaacagagtatgcaaagaaactaaggccagaaag

SEQ ID NO: 506 - DNA, 133 bp, Artificial Sequence
  Synthetic
agcaaacaaacaaacaaacaaacaaactatgacaggaacaaaacgtcacatatcaacattaacaaagaatgtaaacagcctaaatgcttcacttaaaagttatagacaggggctgggcatggtggctcacgcc

SEQ ID NO: 507 - DNA, 77 bp, Artificial Sequence
  Synthetic
aaaagtacagaagacaacaaaaaatgagagagagaaagataacagactatagcagcattggtgatcagagccaccag

SEQ ID NO: 508 - DNA, 137 bp, Artificial Sequence
  Synthetic
tacaagaaaatcacagtaacatttataaaacacagaagtgtgaacacacagctattgaccttgaaaacagtgaaagagggtcagctgtagaactaagacataagcaaagtttttcaatcaagaatacatgggtggcc

SEQ ID NO: 509 - DNA, 118 bp, Artificial Sequence
  Synthetic
gaatcgaatggaatcaacatcaaacggaaaaaaacggaattatcgaatggaatcgaaaagaatcatcgaacggactcgaatggaatcatctaatggaatggaatggaagaatccatgg

SEQ ID NO: 510 - DNA, 73 bp, Artificial Sequence
  Synthetic
aatggaatcgaatggaatcatcatcaaatggaatctaatggaatcattgaacggaattggatggaatcgtcat

SEQ ID NO: 511 - DNA, 105 bp, Artificial Sequence
  Synthetic
aacggaatcaaacggaattatcgaatggaatcgaagagaatcatcgaatggccacgaatggaatcatctaatggaatggaatggaataatccatggacccgaatg

SEQ ID NO: 512 - DNA, 62 bp, Artificial Sequence
  Synthetic
caacatcaaacggaaaaaaacggaattatcgaatggaatcgaagagaatcatcgaatggacc

SEQ ID NO: 513 - DNA, 82 bp, Artificial Sequence
  Synthetic
cacaaccaaagcaatgaaagaaaagcacagacttattgaaatgaaagtacacaccacagaatgggagcaggctcaagcaagc

SEQ ID NO: 514 - DNA, 103 bp, Artificial Sequence
  Synthetic
atcaaagggaatcaagcggaattatcgaatggaatcgaagagaatcatcgaatggactcgaatggaatcatgtgatggaatggaatggaataatccacggact

SEQ ID NO: 515 - DNA, 95 bp, Artificial Sequence
  Synthetic
aagaaacaatcaaaaggaagtgctagaaataaaacacactgtaatagaaaagaagaatgccttatgggcttatcaatagactagacatggccagg

SEQ ID NO: 516 - DNA, 84 bp, Artificial Sequence
  Synthetic
agataagaataaggcaaacatagtaatagggagttcatgaataacacacggaaagagaacttacagggctgtgatcaggaaacg

SEQ ID NO: 517 - DNA, 77 bp, Artificial Sequence
  Synthetic
ggaatcgaatggaatcaatatcaaacggagaaaaacggaattatcgaatggaatcgaagagaatcatcgaatggacc

SEQ ID NO: 518 - DNA, 61 bp, Artificial Sequence
  Synthetic
tcagaccatagcagataacatgcacattagcaatacgattgccatgacagagtggttggtg

SEQ ID NO: 519 - DNA, 73 bp, Artificial Sequence
  Synthetic
aggaatggacacgaacggaatgcaatcgaatggaatggaatctaatagaaaggaattgaatgaaatggactgg

SEQ ID NO: 520 - DNA, 82 bp, Artificial Sequence
  Synthetic
ggaagggaatcaaatgcaacagaatgtaatggaatggaatgcaatggaatgcaatggaatggaatggaatgcaatggaatgg

SEQ ID NO: 521 - DNA, 133 bp, Artificial Sequence
  Synthetic
aaattggattgaatcgaatcgaatggaaaaaatgaaatcaaatgaaattgaatggaatcgaaatgaatgtaaacaatggaatccaatggaatccaatggaatcgaatcaaatggttttgagtggcgtaaaatg

SEQ ID NO: 522 - DNA, 85 bp, Artificial Sequence
  Synthetic
aaggattcgaatggaatgcaatcgaatggaatggaatcgaacggaatggaataaaatggaagaaaactggcaagaaatggaatcg

SEQ ID NO: 523 - DNA, 86 bp, Artificial Sequence
  Synthetic
gaaaaatcattgaacggaatcgaatggaatcatcatcggatggaaacgaatggaatcatcatcgaatggaaatgaaaggagtcatc

SEQ ID NO: 524 - DNA, 92 bp, Artificial Sequence
  Synthetic
ggttcaacttacaatattttgacttgacaacagtgcaaaagcaatacacgattagtagaaacacacttccaatgcccataggaccattctgc

SEQ ID NO: 525 - DNA, 77 bp, Artificial Sequence
  Synthetic
ggaatcgaatggaatcaacatcaaacggagaaaaacggaattatcgaatggaatcgaagagaatcatcgaatggacc

SEQ ID NO: 526 - DNA, 96 bp, Artificial Sequence
  Synthetic
taacctgatttgccataatccacgatacgcttacaacagtgatatacaagttacatgagaaacacaaacattttgcaaggaaactgtggccagatg

SEQ ID NO: 527 - DNA, 102 bp, Artificial Sequence
  Synthetic
taactactcacagaactcaacaaaacactatacatgcatttaccagtttattataaagatacaagtcaggaacagccaaatggaagaaatgtaaatggcaag

SEQ ID NO: 528 - DNA, 95 bp, Artificial Sequence
  Synthetic
gctcaaagaaatcagaaatgacacaagcaaatggaaaaacatgccatgttcatgaatatgaagaatcaatattgttaaaatggccatactgctca

SEQ ID NO: 529 - DNA, 93 bp, Artificial Sequence
  Synthetic
aaagaaatgtcactgcgtatacacacacacgcacatacacacaccatggaatactactcagctatacaaaggaatgaaataatccacagccac

SEQ ID NO: 530 - DNA, 90 bp, Artificial Sequence
  Synthetic
gaatagaacagaatggaatcaaatcgaatgaaatggaatggaatagaaaggaatggaatgaaatggaatggaaaggattcgaatggaatg

SEQ ID NO: 531 - DNA, 76 bp, Artificial Sequence
  Synthetic
tgaacggaatcgaatggaatcatcatcggatggaaacgaatggaatcatcatcgaatggaaatgaaaggagtcatc

SEQ ID NO: 532 - DNA, 60 bp, Artificial Sequence
  Synthetic
gaatagaacgaaatggaatggaatggaatggaatggaaaggaatggaatggaatggaacg

SEQ ID NO: 533 - DNA, 74 bp, Artificial Sequence
  Synthetic
aacgtgacatacatacaaaaagtttttagagcaagtgaaattttagctgctatatgttaattggtggtaatccc

SEQ ID NO: 534 - DNA, 70 bp, Artificial Sequence
  Synthetic
ggaataacaacaacaacaaccaaaagacatatagaaaacaaacagcacgatggcagatgtaaagcctacc

SEQ ID NO: 535 - DNA, 124 bp, Artificial Sequence
  Synthetic
gacaaaaagaatcatcatcgaatagaatcaaatggaatctttgaatggactcaaaaggaatatcgtcaaatggaatcaaaagccatcatcgaatggactgaaatggaattatcaaatggactcg

SEQ ID NO: 536 - DNA, 91 bp, Artificial Sequence
  Synthetic
gtaacaaaacagactcatagaccaatagaacagaatagagaattcagaaataagactgcacttctatgaccatgtgatcttagacaaacct

SEQ ID NO: 537 - DNA, 77 bp, Artificial Sequence
  Synthetic
agataaaaagaacagcagccaaaatgacaaaagcaaaaagcaaaatcgtgttagagccaggtgtggtgatgtgtgct

SEQ ID NO: 538 - DNA, 82 bp, Artificial Sequence
  Synthetic
gcaatctcaggatacaaaatcaatgtgcaaaaatcacaagcattctcatacaccaataacagacaaacagagccaaatcatg

SEQ ID NO: 539 - DNA, 97 bp, Artificial Sequence
  Synthetic
aaccaaaccaagcaaacaaacaaacagtaaaaactcaataacaaccaacaaacaggaaataccaggtaattcagattatctagttatgtgccatagt

SEQ ID NO: 540 - DNA, 148 bp, Artificial Sequence
  Synthetic
gaatgaattgaatgcaaacatcgaatggtctcgaatggaatcatcttcaaatggaatggaatggaatcatcgcatagaatcgaatggaattatcaacgaatggaatcgaatggaatcatcatcagatggaaatgaatggaatcgtcat

SEQ ID NO: 541 - DNA, 97 bp, Artificial Sequence
  Synthetic
tggaatggaatcaaatcgcatggaatcgaatggaatagaaaagaatcaaacagagtggaatggaatggaatggaatggaatcatgccgaatggaatg

SEQ ID NO: 542 - DNA, 87 bp, Artificial Sequence
  Synthetic
gaatccatgttcatagcacaacaaccaaacagaagaaatcactgtgaaataagaaacaaagcaaaacacagatgtcgacacatggca

SEQ ID NO: 543 - DNA, 148 bp, Artificial Sequence
  Synthetic
aaatggaataatgaaatggaatcgaacggaatcatcatcaaaaggaaccgaatgaagtcattgaatggaatcaaaggcaatcatggtcgaatggaatcaaatggaaacagcattgaatagaattgaatggagtcatcacatggaatcg

SEQ ID NO: 544 - DNA, 80 bp, Artificial Sequence
  Synthetic
gaattaacccgaatagaatggaatggaatggaatggaacagaacggaacggaatggaatggaatggaatggaatggaatg

SEQ ID NO: 545 - DNA, 109 bp, Artificial Sequence
  Synthetic
aagatatacaagcagccaacaaacatacgaaagaatgctcaacatcactaatcctcagagaaatttaaatcaaaaccacaatgagttacaatctcataccagtcagaat

SEQ ID NO: 546 - DNA, 106 bp, Artificial Sequence
  Synthetic
agaattacaaaccactgctcaacaaaataaaagagtacacaaacaaatggaagaatattccatgcttatggataggaagaatcaatattgtgaaaatggccatact

SEQ ID NO: 547 - DNA, 93 bp, Artificial Sequence
  Synthetic
catcgaatggactcgaatggaataatcattgaacggaatcgaagggaatcatcatcggatggaaacgaatggaatcatcatcgaatggaaatg

SEQ ID NO: 548 - DNA, 133 bp, Artificial Sequence
  Synthetic
cacccatctgtaggaccaggaagcctgatgtgggagagaacagcaggctaaatccagggttggtctctacagcagagggaatcacaagcctgttagcaagtgaagaaccaacactggcaagagtgtgaaggcc

SEQ ID NO: 549 - DNA, 104 bp, Artificial Sequence
  Synthetic
taatgcaaactaaaacgacaatgagatatcaatacataactaccagaaaggctaacaaaaaaacagtcataacacaccaaaggctgatgagtgaggatgtgcag

SEQ ID NO: 550 - DNA, 105 bp, Artificial Sequence
  Synthetic
aaaggaatcaaacggaattatcgaatggaatcgaaaagaatcatcgaacggactcgaatggaatcatctaatggaatggaatggaagaatccatggactcgaatg

SEQ ID NO: 551 - DNA, 88 bp, Artificial Sequence
  Synthetic
agcaacttcagcaaagtctcaggatacaaaatcaatgagcaaaaatcacaagcattcttacacaccaataacagacaaacagagagcc

SEQ ID NO: 552 - DNA, 92 bp, Artificial Sequence
  Synthetic
ggatataaacaagaaaacaactaatcacaactcaatatcaaagtgcaatgatggtgcaaaatgcaagtatggtggggacagagaaaggatgc

SEQ ID NO: 553 - DNA, 80 bp, Artificial Sequence
  Synthetic
aatcagtaaacgtaatacagcatataaacagaaccaaagacaaaaaccacatgattatctcaatagatgcagaaaaggcc

SEQ ID NO: 554 - DNA, 61 bp, Artificial Sequence
  Synthetic
aacatcaaacggaaaaaaacggaaatatcgaatggaatcgaagagaatcatcgaatggacc

SEQ ID NO: 555 - DNA, 76 bp, Artificial Sequence
  Synthetic
taaaatggaatcgaatggaatcaacatcaaatggaatcaaatggaatcattgaacggaattgaatggaatcgtcat

SEQ ID NO: 556 - DNA, 84 bp, Artificial Sequence
  Synthetic
aatcatcatcgaatggaatcgaatggtatcattgaatggaatcgaatggaatcatcatcagatggaaatgaatggaatcgtcat

SEQ ID NO: 557 - DNA, 64 bp, Artificial Sequence
  Synthetic
caatgcgtcaagctcagacgtgcctcactacggcaatgcgtcaagctcaggcgtgcctcactat

SEQ ID NO: 558 - DNA, 74 bp, Artificial Sequence
  Synthetic
aagacagaacactgaaactcaacagagaagtaacaagaacacctaagacaaggaaggagagggaaggcaggcag

SEQ ID NO: 559 - DNA, 101 bp, Artificial Sequence
  Synthetic
taagctgataagcaactttagcaaagtctcaggatacaaaatcaatgtacaaaaatcacaagcattcttatacaccaacaacagacagacggagagccaaa

SEQ ID NO: 560 - DNA, 114 bp, Artificial Sequence
  Synthetic
atgaacacgaatgtaatgcaatccaatagaatggaatcgaatggcatggaatataaagaaatggaatcgaagagaatggaaacaaatggaatggaattgaatggaatggaattg

SEQ ID NO: 561 - DNA, 84 bp, Artificial Sequence
  Synthetic
aacaatcactagtccttaagtaagagacaacaccttttgtcacacacagtttgtcctaactttatcttggtaattggggagacc

SEQ ID NO: 562 - DNA, 87 bp, Artificial Sequence
  Synthetic
taatgagaagacacagacaacacaaagaatcacagaaacatgacacaggtgacaagaacaggcaaggacctgcagtgcacaggagcc

SEQ ID NO: 563 - DNA, 79 bp, Artificial Sequence
  Synthetic
tgttgagagaaattaaacaaagcacagataaatggaaaaacgtgttcatagattgaaagacttcatgttgtatggtgtc

SEQ ID NO: 564 - DNA, 88 bp, Artificial Sequence
  Synthetic
atcaaacggaatcaaacggaattatcgaatggaatcgaagagaatcatcgaacggactcgaatggaatcatctaatggaatgggatgg

SEQ ID NO: 565 - DNA, 87 bp, Artificial Sequence
  Synthetic
acacaacaaccaagaaacaaccccattaagaagtgggaaaaatacatgaataaacacatctcaaaagaagacaaacaagtggctaac

SEQ ID NO: 566 - DNA, 94 bp, Artificial Sequence
  Synthetic
aatggaaaggaatcaaatggaatataatggaatgcaatggactcgaatggaatggaatggaatggacccaaatggaatggaatggaatggaatg

SEQ ID NO: 567 - DNA, 91 bp, Artificial Sequence
  Synthetic
ggaatacaacggaatggaatcgaaaaaaatggaaaggaatgaaatgaatggaatggaatggaatggaatggatgggaatggaatggaatgg

SEQ ID NO: 568 - DNA, 95 bp, Artificial Sequence
  Synthetic
gaatcaagcggaattatcgaatggaatcgaagagaatcatcgaaaggactcgaatggaatcatctaatggaatggaatggaataatacacggacc

SEQ ID NO: 569 - DNA, 95 bp, Artificial Sequence
  Synthetic
aacaacaacaacaacaggaaaacaacctcagtatgaagacaagtacattgatttattcaacatttactgatcacttttcaggtggtaggcagacc

SEQ ID NO: 570 - DNA, 98 bp, Artificial Sequence
  Synthetic
aagataacctgtgcccaggagaaaaacaatcaatggcaacaaaagcagaaacaacacaaatgatacaattagcagacagaaacattgagattgctatt

SEQ ID NO: 571 - DNA, 88 bp, Artificial Sequence
  Synthetic
aatggactccaatggaataatcattgaacggaatcnaatggaatcatcatcggatggaaatgantggaatcntcntcnaatggaatcn

SEQ ID NO: 572 - DNA, 80 bp, Artificial Sequence
  Synthetic
anncnntaaacgtaatccatcacataaacangancnaanagnnnaaccgcnngattatctcnnnnnntgcnnaaaaggcc

SEQ ID NO: 573 - DNA, 95 bp, Artificial Sequence
  Synthetic
taattgattcgaaattaatggaattgaatggaatgcaatcaaatggaatggaatgtaatgcaatggaatgtaatagaatggaaagcaatggaatg

SEQ ID NO: 574 - DNA, 78 bp, Artificial Sequence
  Synthetic
aaaggaatggacttgaacaaaatgaaatcgaacgataggaatcgtacagaacggaaagaaatggaacggaatggaatg

SEQ ID NO: 575 - DNA, 88 bp, Artificial Sequence
  Synthetic
agcaacttcagcaaaatctcaggatacaaaatcaatgtacaaaaatcacaagcattcttatacaccaacaacagacaaacagagagcc

SEQ ID NO: 576 - DNA, 94 bp, Artificial Sequence
  Synthetic
tgagcagggaacaatgcggataaatttcacaaatacaatgttgagcaaaagaaagacacaaaanaatacacacatacacaccatatgggctagg

SEQ ID NO: 577 - DNA, 85 bp, Artificial Sequence
  Synthetic
aatggaatggaatgtacaagaaaggaatggaatgaaaccgaatggaatggaatggacgcaaaatgaatggaatggaagtcaatgg

SEQ ID NO: 578 - DNA, 129 bp, Artificial Sequence
  Synthetic
aagttcaaacatcagtattaaccttgaacatcaatggcctacatgcatcacttaaaacatacagacaggcaaattgggttaagaaaacaaacaagcaaacaaaacatgttccaaacatttgttggctat

SEQ ID NO: 579 - DNA, 87 bp, Artificial Sequence
  Synthetic
ggaataatcattgaacggaatcgaatggaatcatcatcggatggaaacgaatggaatcatcatcgaatggaaatgaaaggagtcatc

SEQ ID NO: 580 - DNA, 82 bp, Artificial Sequence
  Synthetic
ggaacgaaatcgaatggaacggaatagaatagactcgaatgtaatggattgctatgtaattgattcgaatggaatggaatcg

SEQ ID NO: 581 - DNA, 80 bp, Artificial Sequence
  Synthetic
tgaaaggaatagactggaacaaaatgaaatcgaatggtaggaatcatacagaacagaaagaaatggaacggaatggaatg

SEQ ID NO: 582 - DNA, 75 bp, Artificial Sequence
  Synthetic
aacccgaatagaatggaatggaatggaatggaacggaacggaatggaatggaatggattggaatggaatggaatg

SEQ ID NO: 583 - DNA, 90 bp, Artificial Sequence
  Synthetic
aaagagaatcaaatggaattgaatcgaatggaatcgaatggattggaaaggaatagaatggaatggaatggaatggaatggaatggaatg

SEQ ID NO: 584 - DNA, 106 bp, Artificial Sequence
  Synthetic
aaaacacacaaacatacatgtggatgcacatataaacatgcacatacacacacacataaatgcacaaacacacttaacacaagcacacatgcaaacaaacacatgg

SEQ ID NO: 585 - DNA, 70 bp, Artificial Sequence
  Synthetic
aatggaatcatcagtaatggaatggaaaggaatggaaaggactggaatggaatggaatggaatggaatgg

SEQ ID NO: 586 - DNA, 116 bp, Artificial Sequence
  Synthetic
ggaacaaaatgaaatcgaacggtaggaatcgtacagaacggaaagaaatggaacggaatggaatgcactcaaatggaaaggagtccaatggaatcgaaaggaatagaatggaatgg

SEQ ID NO: 587 - DNA, 132 bp, Artificial Sequence
  Synthetic
agaatgagatcaagcagtataataaaggaagaagtagcaaaattacaacagagcagtgaaatggatatgctttctggcaataattgtgaaaggtctggtaatgagaaagtagcaacagctagtggctgccac

SEQ ID NO: 588 - DNA, 98 bp, Artificial Sequence
  Synthetic
aacaaatggaatcaacatcgaatggaatcgaatggaaacaccatcgaattgaaacgaatggaattatcatgaaattgaaatggatggactcatcatcg

SEQ ID NO: 589 - DNA, 125 bp, Artificial Sequence
  Synthetic
taacatgcagcatgcacacacgaatacacaacacacaaacatgtatgcacgcacacgtgaatacacaacacacacaaacatgcatgcatgcatacatgaatacacagcacacaaatatccagcat

SEQ ID NO: 590 - DNA, 72 bp, Artificial Sequence
  Synthetic
gaatggaatcaacatcaaacggaaaaaaaacggaattatcgaatggaatcgaatagaatcatcgaatggacc

SEQ ID NO: 591 - DNA, 98 bp, Artificial Sequence
  Synthetic
aatcgaatgaaatggagtcaaaaggaatggaatcgaatggcaagaaatcgaatgtaatggaatcgcaaggaattgatgtgaacggaacggaatggaat

SEQ ID NO: 592 - DNA, 91 bp, Artificial Sequence
  Synthetic
aatggaattgaacggaaacatcagcgaatggaatcgaaaggaatcatcatggaatagattcgaatggaatggaaaggaatggaatggaatg

SEQ ID NO: 593 - DNA, 132 bp, Artificial Sequence
  Synthetic
atggaatcaacatcaaacagaatcaaacggaattatcgaatggaatcgaagacaatcatcgaatggactcgaatggaatcatctaatggaatggaatggaagaatccatggtctcgaatgcaatcatcatcg

SEQ ID NO: 594 - DNA, 86 bp, Artificial Sequence
  Synthetic
gaataatcattgaacggaatcgaatggaatcatcttcggatggaaacgaatggaatcatcatcgaatggaaatgaaaggagtcatc

SEQ ID NO: 595 - DNA, 88 bp, Artificial Sequence
  Synthetic
aatggactcgaatggaataatcattgaacggaatcgaatggaatcatcatcggatggaaatgagtggaatcatcatcgaatggaatcg

SEQ ID NO: 596 - DNA, 98 bp, Artificial Sequence
  Synthetic
aaatgaaatcgaacggtaggaatcgtacagaacggaaagaaatggaacggaatggaatgcaatcgaatggaaaggagtccaatggaagggaatcgaat

SEQ ID NO: 597 - DNA, 93 bp, Artificial Sequence
  Synthetic
taccaaacatttaaagaacaaatatcaatcctacgcaaaccattctgaaacacagagatggaggatatacagcgaaactcattctacatggcc

SEQ ID NO: 598 - DNA, 106 bp, Artificial Sequence
  Synthetic
tattggaatggaatggaatggagtcgaatggaacggaatgcactcgaatggaaggcaatgcaatggaatgcactcaacaggaatagaatggaatggaatggaatgg

SEQ ID NO: 599 - DNA, 86 bp, Artificial Sequence
  Synthetic
agagagtattcatcatgaggagtattactggacaaataattcacaaacgaacaaaccaaagcgatcatctttgtactggctggcta

SEQ ID NO: 600 - DNA, 64 bp, Artificial Sequence
  Synthetic
ggaatttaatagaatgtacccgaatggaacggaatggaatggaattgtatggcatggaatggaa

SEQ ID NO: 601 - DNA, 97 bp, Artificial Sequence
  Synthetic
gcaatccantanaatggaatcgaatggcatggaatataaagaaatggaatcgaagagaatggagacaaatggaatggaattgaatggaatggaattg

SEQ ID NO: 602 - DNA, 118 bp, Artificial Sequence
  Synthetic
aatggaatcgaatggaatcatcatcaaatggaatctaatggaatcattgaacggaattaaatggaatcgtcatcgaatgaattcaatgcaatcaacgaatggtctcgaatggaaccac

SEQ ID NO: 603 - DNA, 106 bp, Artificial Sequence
  Synthetic
aattgcaaaagaaacacacatatacacatataaaactcaagaaagacaaaactaacctatggtgatagaaatcagaaaagtacagtacattggttgtcttggtggg

SEQ ID NO: 604 - DNA, 89 bp, Artificial Sequence
  Synthetic
tgacatcattattatcaagaaacattcttaccactgttaccaacttcccaacacagactatggagagagagataagacagaatagcatt

SEQ ID NO: 605 - DNA, 81 bp, Artificial Sequence
  Synthetic
ggaatctataatacagctgtttatagccaagcactaaatcatatgatacagaaaacaaatgcagatggtttgaagggtggg

SEQ ID NO: 606 - DNA, 89 bp, Artificial Sequence
  Synthetic
aaagaattgaattgaatagaatcaccaatgaattgaatcgaatggaatcgtcatcgaatggaatcgaagggaatcattggatgggctca

SEQ ID NO: 607 - DNA, 87 bp, Artificial Sequence
  Synthetic
atcatcgaatggaatcgaatggaatcaatatcaaacggaaaaaaacggaattatcgaatggaatcgaatagaatcatcgaatggacc

SEQ ID NO: 608 - DNA, 83 bp, Artificial Sequence
  Synthetic
gaatgaaatcgtatagaatcatcgaatgcaactgaatggaatcattaaatggacttgaaaggaattattatggaatggaattg

SEQ ID NO: 609 - DNA, 90 bp, Artificial Sequence
  Synthetic
taagcaacttcagcaaagtctcaggatacaaaatcaatgtgcaaaaatctcaagcattcttatacacgaacaacagacaaacagagagct

SEQ ID NO: 610 - DNA, 79 bp, Artificial Sequence
  Synthetic
actcaaaaggaattgattcgaatggaatagaatggcaaggaatagtattgaattgaatggaatggaatggacccaaatg

SEQ ID NO: 611 - DNA, 95 bp, Artificial Sequence
  Synthetic
gaatggaatttaaaggaatagaatggaaggaatcggatggaatggaatggaatagaatggagtcgaatggaatagaatcgaatggaatggcattg

SEQ ID NO: 612 - DNA, 86 bp, Artificial Sequence
  Synthetic
aacaaaaaatgagtcaagccttaaataaaatcagagccaaaaaagaagacattacatctgataagacaaaaattcaaaggaccatc

SEQ ID NO: 613 - DNA, 96 bp, Artificial Sequence
  Synthetic
aacccagtggaattgaattgaatggaattgaatggaatggaaagaatcaatccgagtcgaatggaatggtatggaatggaatggcatggaatcaac

SEQ ID NO: 614 - DNA, 65 bp, Artificial Sequence
  Synthetic
atcaacatcaaacggaaaaaaaacggaattatcgaatggaatcgaagagaatcatcgaatggacc

SEQ ID NO: 615 - DNA, 92 bp, Artificial Sequence
  Synthetic
aaggaatggaatggtacggaatagaatggaatggaacgaattgtaatggaatggaatttaatggaacggaatggaatggaatggaatcaacg

SEQ ID NO: 616 - DNA, 66 bp, Artificial Sequence
  Synthetic
aacggaatggaaagcaatttaatcaaatgcaatacagtggaattgaagggaatggaatggaatggc

SEQ ID NO: 617 - DNA, 150 bp, Artificial Sequence
  Synthetic
aatcgaatggaacggaatagaatagactcgaatgtaatggattgctatgtaattgattcgaatggaatggaatcgaatggaatgcaatccaatggaatggaatgcaatgcaatggaatggaatcgaacggaatgcagtggaagggaatgg

SEQ ID NO: 618 - DNA, 87 bp, Artificial Sequence
  Synthetic
tagcaacattttagtaacatgatagaaacaaaacagcaacatagcaatgcaatagtaacacaacagcaacatcataacatggcagca

SEQ ID NO: 619 - DNA, 88 bp, Artificial Sequence
  Synthetic
ggacaaattgctagaaataaacaaattaccaaaaatgattcaagtagagacagagaatcaaaatagaactacacataagtgggccaag

SEQ ID NO: 620 - DNA, 86 bp, Artificial Sequence
  Synthetic
aaaatagaatgaaagagaatcaaatggaattgaatcgaatggaatcgaatggattggaaaggaatagaatggaatggaatggaatg

SEQ ID NO: 621 - DNA, 136 bp, Artificial Sequence
  Synthetic
agcaaacaagtgaataaacaagcaaacaagtgaacaagcaaacaagtgaataaacaagcaaacaagtgaacaagcaaacaagtgaataaacaagcaaacaagtgaacaaggaaacaagtgaataaacaaaggctct

SEQ ID NO: 622 - DNA, 94 bp, Artificial Sequence
  Synthetic
aatggaatcaacacgagtgcaattgaatggaatcgaatggaatggaatggaatggaatgaattcaacccgaatggaatggaaaggaatggaatc

SEQ ID NO: 623 - DNA, 90 bp, Artificial Sequence
  Synthetic
aatatacgcaaatcaataaatgtaatccagcatataaacagtactaaagacaaaaaccacatgattatctcaatagatgcagaaaaggcc

SEQ ID NO: 624 - DNA, 76 bp, Artificial Sequence
  Synthetic
gaatcgaatggaatcaacatcaaacggaaaaaaacggaattatcgaatggaatcgaagagnnnnnncgaatggacc

SEQ ID NO: 625 - DNA, 141 bp, Artificial Sequence
  Synthetic
aacacgaatgtaatgcaatccaatagaatggaatcgaatggcatggaatataaagaaatggaatcgaagagaatggaaacaaacggaatggaattgaatggaatggaattgaatggaatgggaacgaatggagtgaaattg

SEQ ID NO: 626 - DNA, 146 bp, Artificial Sequence
  Synthetic
gaatggaacggaatagaacagactcgaatgtaatggattgctatgtaattgattcgaatggaatggaatcgaatggaatgcaatccaatggaatggaatgcaatgcaatggaatggaatcgaatggaatgcagtggaagggaatgg

SEQ ID NO: 627 - DNA, 76 bp, Artificial Sequence
  Synthetic
gaatcgaatggaatcaatatcaaacggaaaaaaacggaattatcgaatggaatcgaagagaatcatcgaatggacc

SEQ ID NO: 628 - DNA, 82 bp, Artificial Sequence
  Synthetic
taaacaacgagaacacatgaacacaaagaggggaacaacagacaccaagaccttcttgagggtggaggatgggaggagggag

SEQ ID NO: 629 - DNA, 86 bp, Artificial Sequence
  Synthetic
agcaacttcagcagtctcagtatacaaaaacaatgtgcaaaaatcacaagcattcctatatgccaataacagacaaacagagagcc

SEQ ID NO: 630 - DNA, 95 bp, Artificial Sequence
  Synthetic
atcaaaagaaaagcaacctaacaaatacgggaagaatatttgaatagacatttcacaggaaaagatatatgaatggccaaaaagcaaatgaaaag

SEQ ID NO: 631 - DNA, 136 bp, Artificial Sequence
  Synthetic
ataaacatcaaacggaatcaaacggaattatcgaatggaatcgaagagaataatcgaatggactcaaatggagtcatctaatggaatggtatggaagaatccatggactccaacgcaatcatcagcgaatggaatc

SEQ ID NO: 632 - DNA, 101 bp, Artificial Sequence
  Synthetic
aaaagaaaagacaaaagacaccaattgccaatactgaaatgaaaaaacaggtaataactattgatcccatggacattaaaatgatgttgaaggaacaccac

SEQ ID NO: 633 - DNA, 92 bp, Artificial Sequence
  Synthetic
agcaataaccaaacaacctcattaaaaagtaggcaaaggacataaacagacacttttcaaaagaagacatacacgtggccaacaaacatatg

SEQ ID NO: 634 - DNA, 88 bp, Artificial Sequence
  Synthetic
agcaacttcagcaaagtctcaggatacaaaatcgatgtgcaaaaatcacaagcattcttatacaccaataacaggcaaacagagagcc

SEQ ID NO: 635 - DNA, 77 bp, Artificial Sequence
  Synthetic
gtcatatttgggatttatcatctgtttctattgttgttgttttagtacacacaaagccacaataaatattctaggct

SEQ ID NO: 636 - DNA, 87 bp, Artificial Sequence
  Synthetic
atcatcgaatggaatagaatggtatcaacatcaaacggagaaaaacggaattatcgaatggaatcgaagagaatcttcgaacggacc

SEQ ID NO: 637 - DNA, 97 bp, Artificial Sequence
  Synthetic
aaataagccaacggtcataaattgcaaagccttttacaatccaaacatgatggaaacgatatgccattttgaaggtgatttgaaaagcacatggttt

SEQ ID NO: 638 - DNA, 122 bp, Artificial Sequence
  Synthetic
gaatggaatcatcgcatagaatcggatggaattatcatcgaatggaatcgaatggtatcaacatcaaacggaaaaaaacggaattatcgaatggaatcgaattgaatcatcgaacggacccg

SEQ ID NO: 639 - DNA, 133 bp, Artificial Sequence
  Synthetic
aatggactcgaatggaataatcattgaacggaatcgaatggaatcatcatcggatggaaatgaatggaataatccatggactcgaatgcaatcatcatcgaatggaatcgaatggaatcatcgaatggactcg

SEQ ID NO: 640 - DNA, 70 bp, Artificial Sequence
  Synthetic
aatgcaatcatcaactggcttcgaatggaatcatcaagaatggaatcgaatggaatcatcgaatggactc

SEQ ID NO: 641 - DNA, 68 bp, Artificial Sequence
  Synthetic
aagagaccaataaggantangtaagcaacangaggaaggaganangggcaagagagatgaccagagtt

SEQ ID NO: 642 - DNA, 87 bp, Artificial Sequence
  Synthetic
tggaatcatcataaaatggaatcgaatggaatcaacatcaaatggaatcaaatggaatcattgaacggaattgaatggaatcgtcat

SEQ ID NO: 643 - DNA, 117 bp, Artificial Sequence
  Synthetic
ggaatcatcgcatagaatcgaatggaattatcatcgaatggaatcgaatggaatcaacatcaaacgaaaaaaaaccggaattatcgaatggaatcgaagagaatcatcgaacggacc

SEQ ID NO: 644 - DNA, 85 bp, Artificial Sequence
  Synthetic
aaatcatcatcgaatgggatcgaatggtatccttgaatggaatcgaatggaatcatcatcagatggaaatgaatggaatcgtcat

SEQ ID NO: 645 - DNA, 104 bp, Artificial Sequence
  Synthetic
ggaatgtaatagaacggaaagcaatggaatggaacgcactggattcgagtgcaatggaatctattggaatggaatcgaatggaatggtttggcatggaatggac